(19) United States
(12) Patent Application Publication
(10) Pub. No.: US 2007/0203118 A1
(43) Pub. Date: Aug. 30, 2007

(54) SUBSTITUTED TETRAHYDROISOQUINOLINES USED IN THE FORM OF MMP INHIBITORS, METHOD FOR THE PRODUCTION AND USE THEREOF IN THE FORM OF DRUGS

(75) Inventors: Armin Hofmeister, Dexheim (DE); Manfred Schudok, Eppstein/Ts (DE); Hans Matter, Langenselbold (DE); Kristin Breitschopf, Offenbach (DE); Antonio Ugolini, Massy (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 11/612,938

(22) Filed: Dec. 19, 2006

Related U.S. Application Data
(63) Continuation of application No. PCT/EP05/06415, filed on Jun. 15, 2005.

(30) Foreign Application Priority Data
Jun. 30, 2004 (DE) 102004031850.6

(51) Int. Cl. A61K 31/55; A61K 31/4704; A61K 31/4035; C07D 217/22; C07D 223/16; C07D 209/44

(52) U.S. Cl. 514/217.01; 514/309; 514/416; 540/593; 546/141; 548/470

(57) ABSTRACT

The present invention is directed to a compound of formula (I) 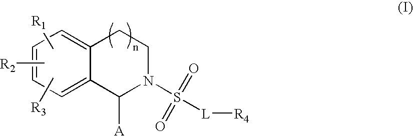
wherein R₁, R₂, R₃, R₄, A, L and n are as defined herein, or a pharmacologically acceptable salt thereof, its pharmaceutical composition and it use as a MMP inhibitor.

This application is a Continuation of International Application No. PCT/EP2005/006415, filed Jun. 15, 2005.

### BACKGROUND OF THE INVENTION

In diseases such as osteoarthritis and rheumatism there is destruction of the joint caused in particular by the proteolytic breakdown of collagen by collagenases. Collagenases belong to the superfamily of metalloproteinases (MP) or matrix metallproteinases (MMP or MMPS). The MMPs form a group of Zn-dependent enzymes involved in the biodegradation of the extracellular matrix (D. Yip et al., Investigational New Drugs 1999, 17, 387-399 and Michaelides et al., Current Pharmaceutical Design 1999, 5, 787-819). These MMPs are capable in particular of breaking down fibrillary and non-fibrillary collagen, and proteoglycans, both of which represent important matrix constituents. MMPs are involved in processes of wound healing, of tumor invasion, metastasis migration and in angiogenesis, multiple sclerosis and heart failure (Michaelides et al., ibid, page 788). In particular they play an important part in the breakdown of the joint matrix in arthrosis and arthritis, whether osteoarthrosis, osteoarthritis or rheumatoid arthritis.

The activity of MMPs is moreover essential for many of the processes involved in atherosclerotic plaque formation, such as infiltration of inflammatory cells, smooth muscle migration, and proliferation and angiogenesis (S. J. George, Exp. Opin. Invest. Drugs 2000, 9 (5), 993-1007). Moreover, matrix degradation by MMP may cause plaque instabilities or even ruptures, possibly leading to the signs and symptoms of atherosclerosis, unstable angina pectoris, myocardial infarction or stroke (E. J. M. Creemers et al., Circulation Res. 2001, 89, 201-210). Considered overall, the entire MMP family can break down all the components of the extracellular matrix of the blood vessels; their activity is therefore subject in a high degree to regulatory mechanisms in normal blood vessels. Elevated MMP activity during plaque formation and plaque instability is caused by increased cytokine- and growth factor-stimulated gene transcription, increased zymogen activation and an imbalance in the MMP-TIMP ratio (tissue inhibitors of metalloproteases). MMP inhibition or restoration of the MMP-TIMP balance is therefore of assistance in the treatment of atherosclerotic disorders. In addition, besides atherosclerosis, other cardiovascular disorders are also at least partly caused by an elevated MMP activity, such as, for example, restenosis, dilated cardiomyopathy and the myocardial infarction which has already been mentioned. It has been possible to show in experimental animal models of these disorders that distinct improvements can be achieved by administration of synthetic MMP inhibitors, e.g. relating to the formation of atherosclerotic lesions, neointima formation, left ventricular remodeling, dysfunction of pumping efficiency or healing of infarctions. Detailed tissue analysis in various preclinical studies with MMP inhibitors showed reduced collagen damage, improved extracellular matrix remodeling and an improved structure and function of myocardium and vessels. Of these processes, in particular matrix remodeling processes and MMP-regulated fibroses are regarded as important components in the progression of heart diseases (infarction) (Drugs 2001, 61, 1239-1252).

MMPs cleave matrix proteins such as collagen, laminin, proteoglycans, elastin or gelatin, and MMPs moreover process (i.e. activate or deactivate) by cleavage a large number of other proteins and enzymes under physiological conditions, so that they are important in the whole body, with particular importance in connective tissue and bone.

A large number of different MMP inhibitors are known (Current Medicinal Chemistry 2001, 8, 425-474).

DE19542189 describes compounds of type (A) 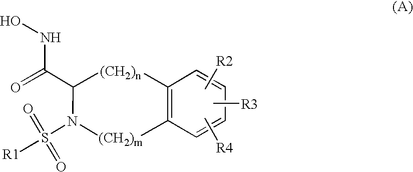
in which n and m may each be 0, 1 or 2, R1 is R5—X-Ph-A- with A=(C₁-C₄)alkyl or —CH═CH—, X=a covalent bond, —O—, —S—, —C(O)—, —NH—, —N(C₁-C₄)alkyl. WO9718194 describes compounds of type (B) 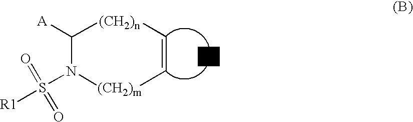
where the substitutent A may be C(O)NHOH or C(O)OH,
Q may be a phenyl ring which is substituted zero to three times by radicals R6, R7, R8,
n and m are each 0, 1 or 2, and
R1 may be 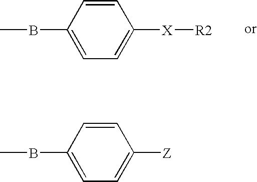
where
B may be —(CH₂)_{q}— with q=1, 2, 3 or 4,
X may be a covalent bond, —O—, —S—, —C(O)—,
R2 is a phenyl radical which is substituted zero to three times, and
Z is a radical of a heterocycle or substituted heterocycle.

WO03016248 describes MMP and/or TACE inhibitors of the formula (C) 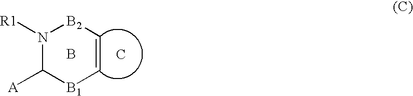
in which the B-C ring system is described, inter alia, by a group 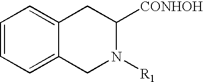
in which the hydroxamic acid function A is in the 3 position.

Ma et al. (Bioorg. Med. Chem. Lett. 2004, 14, 47-50) describe the synthesis of tetrahydroisoquinoline-1-hydroxamic acids of the formula (D), which inhibit in particular MMP-1, MMP-12, MMP-15 and MMP-16, 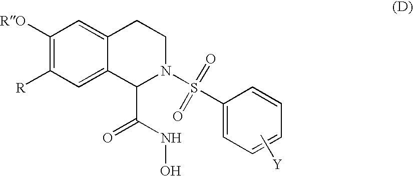
where
Y is H, methyl, methoxy, NH₂, NO₂ or Cl,
R is H or OCH₃, and
OR″ is H or benzyl,
   none of the compounds (D) having a substitutent R or OR″ in position 8 of the tetrahydroisoquinoline.

The Chinese patent application CN1380288A describes N-hydroxytetrahydro-isoquinolinecarboxamide derivatives of the formula (E) 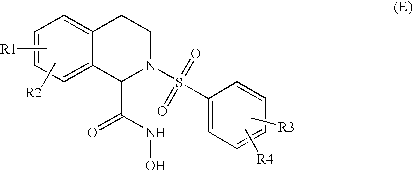
where
R1 and/or R2 are H, OH, O(C₁-C₁₂)alkyl or O(C₁-C₁₂)aryl, and
R3 and/or R4 are H, OH, O(C₁-C₁₂)alkyl or O(C₁-C₁₂)aryl, Br, Cl, NO₂, NH₂, (C₁-C₁₂)alkyl or (C₁-C₁₂)aryl,
   and where none of the compounds (E) has a substitutent R1 or R2 in position 8 of the tetrahydroisoquinoline.

It has emerged from initial clinical studies on humans that MMP inhibitors cause side effects. The side effects which are chiefly mentioned are musculoskeletal pain or anthralgias. It is unambiguous from the prior art that it is expected that selective inhibitors will be able to reduce these side effects mentioned (D. Yip et al., Investigational New Drugs 1999, 17, 387-399). Specificity in relation to MMP-1 should be particularly emphasized in this connection, because these unwanted side effects evidently occur to an increased extent with inhibition of MMP-1.

A disadvantage of known MMP inhibitors is therefore frequently their lack of specificity. Most MMP inhibitors inhibit many MMPs simultaneously because of the similarity in structure of the catalytic domain of the various MMP subtypes. Accordingly, the inhibitors act in an unwanted way on the enzymes, including those with a vital function (Massova I, et al., The FASEB Journal 1998, 12, 1075-1095).

In the attempt to find effective compounds for the treatment of the above-mentioned disorders, it has now been found that the inventive compounds of the formula (I) are strong inhibitors of matrix metalloproteinases MMP-2 and MMP-9, and show only a weak inhibition of MMP-1.

### SUMMARY OF THE INVENTION

The present invention therefore relates to a compound of the formula (I) 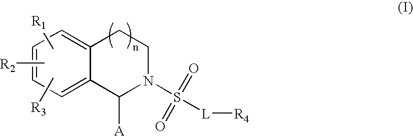
where R₁, R₂ and R₃
are independently of one another H, F, Cl, Br, I, NO₂, CN, OH, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₃-C₈)cycloalkyl, O(C₁-C₄)alkyl-(C₃-C₈)cycloalkyl, O(C₃-C₈)cycloalkyl-(C₁-C₄)alkyl, OC(O)—(C₁-C₆)alkyl, OC(O)—(C₂-C₆)alkenyl, OC(O)—(C₃-C₈)cycloalkyl, OC(O)—(C₁-C₄)alkyl-(C₃-C₉)cycloalkyl, OC(O)—(C₃-C₉)cycloalkyl-(C₁-C₄)alkyl, C(O)O—(C₁-C₆)alkyl, C(O)O—(C₂-C₆)alkenyl, C(O)O—(C₃-C₈)cycloalkyl, C(O)O—(C₁-C₄)alkyl-(C₃-C₈)cycloalkyl, C(O)O—(C₃-C₈)cycloalkyl-(C₁-C₄)alkyl, C(O)NR₆R₇, NR₆R₇ or NR₆C(O)R₇, where
R₆ and R₇ are independently of one another H or (C₁-C₆)alkyl,
A is C(O)R₅ or CH₂SH, where
   R₅ is OR₆, NR₆R₇ or NR₆OH,
n is 0, 1 or 2;
L is defined by —O—, —NR₁₄—, a covalent bond or —(CH₂)_{q}—, where
   R₁₄ is defined by H or (C₁-C₆)alkyl, and
   q is 1, 2, 3 or 4, and
R₄ is phenyl or (C₅-C₁₄)heteroaryl, where the phenyl or (C₅-C₁₄)heteroaryl radical is optionally substituted by 1, 2 or 3 substitutents independently of one another selected from the group of F, Cl, Br, I, CN, OH, NO₂, (C₁-C₆)alkyl, (C₂-C₆)alkenyl, (C₂-C₆)alkynyl, (C₃-C₈)cycloalkyl, (C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₂-C₆)alkynyl, O(C₃-C₈)cycloalkyl, O(C₁-C₄)alkyl-(C₃-C₈)cycloalkyl, O(C₃-C₈)cycloalkyl-(C₁-C₄)alkyl, —O(C₁-C₄)alkyl-O—(C₁-C₆)alkyl or a radical NR₈R₉, where
   R₈ and R₉ are defined independently of one another by H, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, (C₂-C₆)alkynyl, C(O)—V—(C₁-C₆)alkyl, C(O)—V—(C₂-C₆)alkenyl, C(O)—V—(C₃-C₈)cycloalkyl, C(O)—V—(C₁-C₄)alkyl-(C₃-C₈)cycloalkyl, C(O)—V—(C₃-C₈)cycloalkyl-(C₁-C₄)alkyl or C(O)—V—(C₂-C₆)alkynyl, where
      V is a covalent bond, —O— or —NH—, and where
      R₈ and R₉ may together form a 5- or 6-membered ring,
   and where the phenyl or (C₅-C₁₄)heteroaryl radical is optionally substituted by a group T-Z, where
   T is defined by a covalent bond, —O—, —S—, —O(C₁-C₄)alkyl-, —N(R₁₀)—, —C(O)—, —C(O)O—, —OC(O)—, —C(O)N(R₁₀)—, —N(R₁₀)—C(O)— or —N(R₁₀)—C(O)—N(R₁₁)—, where
      R₁₀ and R₁₁ are independently of one another H or (C₁-C₄)alkyl,
   Z is selected from the group of phenyl, (C₅-C₁₄)heteroaryl, (C₃-C₈)heterocycloalkyl or benzocyclo(C₅-C₇)alken-1-one,
   where phenyl, benzocyclo(C₅-C₇)alken-1-one, (C₅-C₁₄)heteroaryl or (C₃-C₈)heterocycloalkyl are unsubstituted or substituted by 1, 2 or 3 substitutents independently of one another selected from the group of F, Cl, Br, I, CN, OH, NO₂, (C₁-C₆)alkyl, SO₂(C₁-C₆)alkyl, O(C₁-C₄)alkyl-O—(C₁-C₆)alkyl, —(C₁-C₄)alkyl-C(O)—O(C₁-C₆)alkyl, O(C₁-C₆)alkyl, (C₂-C₆)alkenyl, (C₃-C₉)cycloalkyl, —(C₁-C₄)alkyl-(C₃-C₈)cycloalkyl or —(C₃-C₈)cycloalkyl-(C₁-C₄)alkyl, where one or more CH₂ groups in the alkenyl, alkyl or cycloalkyl radicals may be replaced by O or C(O), or else O(C₂-C₆)alkenyl, O(C₃-C₈)cycloalkyl, O(C₁-C₄)alkyl-(C₃-C₈)cycloalkyl, O(C₃-C₈)cycloalkyl-(C₁-C₄)alkyl, (C₂-C₆)alkynyl, O(C₂-C₆)alkynyl, or NR₁₂R₁₃, where
      R₁₂ and R₁₃ are defined independently of one another by H, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, (C₂-C₆)alkynyl, C(O)—W—(C₁-C₆)alkyl, C(O)—W—(C₂-C₆)alkenyl, C(O)—W—(C₃-C₈)cycloalkyl, C(O)—W—(C₁-C₄)alkyl-(C₃-C₈)cycloalkyl, C(O)—W—(C₃-C₈)cycloalkyl-(C₁-C₄)alkyl, or C(O)—W—(C₂-C₆)alkynyl, where
      W is a covalent bond, —O— or —NH—;
         where one or more H atoms in (C₁-C₆)alkyl, (C₁-C₄)alkyl, (C₂-C₆)alkenyl, (C₃-C₈)cycloalkyl or (C₂-C₆)alkynyl radicals may optionally be replaced independently of one another by F atoms,
         excluding compounds in which
R₁, R₂ and R₃ are H,
A is C(O)OH,
n is 1,
L is a covalent bond, and
R₄ is a 4-(4′-chlorobiphenyl radical); or
R₁ and R₂ are O-methyl,
R₃ is H,
A is C(O)OH,
n is 1,
L is a covalent bond, and
R₄ is a 4-(4′-chlorobiphenyl radical); or
R₁ is OH or O-benzyl,
R₂ is H or O-methyl,
R₃ is H,
A is C(O)NHOH,
n is 1,
L is a covalent bond, and
R₄ is a phenyl radical which is unsubstituted or substituted by methyl, methoxy, NH₂, NO₂ or Cl; or
R₁ and R₂ are H, OH or O(C₁-C₆)alkyl,
R₃ is H,
A is C(O)NHOH,
n is 1,
L is a covalent bond, and
R₄ is a phenyl radical which is unsubstituted or substituted by H, OH, O(C₁-C₆)alkyl or O(C₁-C₁₂)aryl, Br, C₁, NO₂, NH₂, (C₁-C₆)alkyl or (C₁-C₁₂)aryl;
   and the pharmacologically acceptable salts thereof.

### DETAILED DESCRIPTION OF THE INVENTION

### Particular Embodiments of the Invention

The invention preferably relates to a compound of the formula (I) where
R₁, R₂, R₃, A and L are as defined above,
n is 1, and
R₄ is a pyridyl radical, where the pyridyl radical is optionally substituted by 1, 2 or 3 substitutents independently of one another selected from the group of F, Cl, Br, I, CN, OH, NO₂, (C₁-C₆)alkyl, (C₂-C₆)alkenyl, (C₂-C₆)alkynyl, (C₃-C₈)cycloalkyl, (C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₂-C₆)alkynyl, O(C₃-C₈)cycloalkyl, O(C₁-C₄)alkyl-(C₃-C₈)cycloalkyl, O(C₃-C₈)cycloalkyl-(C₁-C₄)alkyl, —O(C₁-C₄)alkyl-O—(C₁-C₆)alkyl or a radical NR₈R₉, where
   R₈ and R₉ are defined independently of one another by H, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, (C₂-C₆)alkynyl, C(O)—V—(C₁-C₆)alkyl, C(O)—V—(C₂-C₆)alkenyl, C(O)—V—(C₃-C₈)cycloalkyl, C(O)—V—(C₁-C₄)alkyl-(C₃-C₈)cycloalkyl, C(O)—V—(C₃-C₈)cycloalkyl-(C₁-C₄)alkyl or C(O)—V—(C₂-C₆)alkynyl, where
      V is a covalent bond, —O— or —NH—, and where
      R₈ and R₉ may together form a 5- or 6-membered ring,
   and where the pyridyl radical is optionally substituted by a group T-Z, where
   T is defined by a covalent bond, —O—, —S—, —O(C₁-C₄)alkyl-, —N(R₁₀)—, —C(O)—, —C(O)O—, —OC(O)—, —C(O)N(R₁₀)—, —N(R₁₀)—C(O)— or —N(R₁₀)—C(O)—N(R₁₁)—, where
      R₁₀ and R₁₁ are independently of one another H or (C₁-C₄)alkyl,
   Z is selected from the group of phenyl, (C₅-C₁₄)heteroaryl, (C₃-C₈)heterocycloalkyl or benzocyclo(C₅-C₇)alken-1-one,
   where phenyl, benzocyclo(C₅-C₇)alken-1-one, (C₅-C₁₄)heteroaryl or (C₃-C₈)heterocycloalkyl are unsubstituted or substituted by 1, 2 or 3 substitutents independently of one another selected from the group of F, Cl, Br, I, CN, OH, NO₂, (C₁-C₆)alkyl, SO₂(C₁-C₆)alkyl, O(C₁-C₄)alkyl-O—(C₁-C₆)alkyl, —(C₁-C₄)alkyl-C(O)—O(C₁-C₆)alkyl, O(C₁-C₆)alkyl, (C₂-C₆)alkenyl, (C₃-C₈)cycloalkyl, —(C₁-C₄)alkyl-(C₃-C₈)cycloalkyl or —(C₃-C₈)cycloalkyl-(C₁-C₄)alkyl where one or more CH₂ groups in the alkenyl, alkyl or cycloalkyl radicals may be replaced by O or C(O), or else O(C₂-C₆)alkenyl, O(C₃-C₈)cycloalkyl, O(C₁-C₄)alkyl-(C₃-C₈)cycloalkyl, O(C₃-C₈)cycloalkyl-(C₁-C₄)alkyl, (C₂-C₆)alkynyl, O(C₂-C₆)alkynyl, or NR₁₂R₁₃, where
      R₁₂ and R₁₃ are defined independently of one another by H, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, (C₂-C₆)alkynyl, C(O)—W—(C₁-C₆)alkyl, C(O)—W—(C₂-C₆)alkenyl, C(O)—W—(C₃-C₈)cycloalkyl, C(O)—W—(C₁-C₄)alkyl-(C₃-C₈)cycloalkyl, C(O)—W—(C₃-C₈)cycloalkyl-(C₁-C₄)alkyl, or C(O)—W—(C₂-C₆)alkynyl, where
      W is a covalent bond, —O— or —NH—;
         and the pharmacologically acceptable salts thereof.

The invention further preferably relates to a compound of the formula (I) where
R₁, R₂ and R₃ are independently of one another H, F, Cl, Br, OH, NO₂, (C₁-C₆)alkyl, O(C₁-C₆)alkyl,
A is C(O)NHOH,
L is defined by a covalent bond or —(CH₂)_{q}— where
   q is 1 or 2;
R₄ is phenyl or pyridyl,
   where phenyl or pyridyl is unsubstituted or substituted by 1, 2 or 3 radicals independently of one another selected from the group of F; Cl; (C₁-C₆)alkyl, preferably methyl or ethyl; O(C₁-C₆)alkyl, preferably O-methyl;
   where phenyl or pyridyl are either substituted by a group NR₈R₉ where R₈ and R₉ are independently of one another H or (C₁-C₆)alkyl, preferably by the radical N(CH₃)₂,
   or is substituted by a group T-Z where
   T is defined by a covalent bond; —O—; —S—; —O—(C₁-C₄)alkyl-, preferably —O—CH₂—; or —O—C(O)—, and
   Z is selected from the group of phenyl; (C₅-C₁₀)heteroaryl, preferably pyridyl, pyrazolyl or indolyl; C₅-C₇-heterocycloalkyl, particularly preferably morpholinyl; benzocyclo(C₅-C₇)alken-1-on-yl, preferably indan-1-on-yl;
   where phenyl, (C₅-C₁₀)heteroaryl, C₅-C₇-heterocycloalkyl and benzocyclo(C₅-C₇)alken-1-on-yl are unsubstituted or are substituted by 1, 2 or 3 substitutents independently of one another selected from the group of F; Cl; Br; CN; OH;
   (C₁-C₆)alkyl, where one or more H atoms may be replaced by F atoms, preferably CF₃; —SO₂(C₁-C₆)alkyl, preferably —SO₂CH₃;
   O(C₁-C₆)alkyl, where one or more H atoms may be replaced by F atoms, preferably OMe, OEt, O(CH₂)₃CH₃, OCF₃ or OCH₂CF₃;
   —CH₂—C(O)—O(C₁-C₆)alkyl, preferably —CH₂—C(O)—OMe or —CH₂—C(O)—OEt; —O—(C₁-C₄)alkyl-O—(C₁-C₆)alkyl, preferably —O(CH₂)₂OCH₃; NR₁₂R₁₃, where R₁₂ and R₁₃ are defined independently of one another by H, C(O)—W—(C₁-C₆)alkyl, or C(O)—W—(C₂-C₆)alkynyl, where W is a covalent bond or —O—, preferably NHC(O)O(i-Pr), NHC(O)OCH₂C≡CCH₃ or NHC(O)CH₃; and
n is 1,
   with exclusion of those compounds in which
R₁ and R₂ are H, OH or O(C₁-C₆)alkyl,
R₃ is H,
A is C(O)NHOH,
n is 1,
L is a covalent bond, and
R₄ is a phenyl radical which is substituted by O(C₁-C₁₂)aryl, NH₂ or (C₁-C₁₂)aryl, and is optionally substituted by O(C₁-C₆)alkyl, (C₁-C₆)alkyl, Cl;
   and the pharmacologically acceptable salts thereof.

The invention further preferably relates to a compound of the formula (I) where
R₁, R₂ and R₃ are independently of one another H, F, Cl, Br, OH, NO₂, (C₁-C₆)alkyl, O(C₁-C₆)alkyl,
A is C(O)NHOH,
L is defined by a covalent bond or —(CH₂)_{q}—, where
   q is 1 or 2;
R₄ is a pyridyl radical, where the pyridyl radical is unsubstituted or substituted by 1, 2 or 3 radicals independently of one another selected from the group of F; Cl; (C₁-C₆)alkyl, preferably methyl or ethyl; O(C₁-C₆)alkyl, preferably O-methyl;
   where pyridyl is either substituted by a group NR₈R₉ where
   R₈ and R₉ are independently of one another H or (C₁-C₆)alkyl, preferably by a radical N(CH₃)₂,
   or pyridyl is substituted by a group T-Z where
   T is defined by a covalent bond; —O—; —S—; —O—(C₁-C₄)alkyl, preferably —O—CH₂—; or —O—C(O)—, and
   Z is selected from the group of phenyl; (C₅-C₁₀)heteroaryl, preferably pyridyl, pyrazolyl or indolyl; C₅-C₇-heterocycloalkyl, particularly preferably morpholinyl; benzocyclo(C₅-C₇)alken-1-on-yl, preferably indan-1-on-yl; where phenyl, (C₅-C₁₀)heteroaryl, C₅-C₇-heterocycloalkyl and benzocyclo(C₅-C₇)alken-1-on-yl are unsubstituted or are substituted by 1, 2 or 3 substitutents independently of one another selected from the group of F; Cl; Br; CN; OH;
   (C₁-C₆)alkyl, where one or more H atoms may be replaced by F atoms, preferably CF₃; —SO₂(C₁-C₆)alkyl, preferably —SO₂CH₃;
   O(C₁-C₆)alkyl, where one or more H atoms may be replaced by F atoms, preferably OMe, OEt, O(CH₂)₃CH₃, OCF₃ or OCH₂CF₃;
   —CH₂—C(O)—O(C₁-C₆)alkyl, preferably —CH₂—C(O)—OMe or —CH₂—C(O)—OEt; —O—(C₁-C₄)alkyl-O—(C₁-C₆)alkyl, preferably —O(CH₂)₂OCH₃; NR₁₂R₁₃, where R₁₂ and R₁₃ are defined independently of one another by H, C(O)—W—(C₁-C₆)alkyl, or C(O)—W—(C₂-C₆)alkynyl, where W is a covalent bond or —O—, preferably NHC(O)O(i-Pr), NHC(O)OCH₂C≡CCH₃ or NHC(O)CH₃; and
n is 1;
   and the pharmacologically acceptable salts thereof.

The invention further relates particularly preferably to a compound of the formula (I) where
R₁, R₂ and R₃ are independently of one another H; F; NO₂; (C₁-C₆)alkyl, preferably methyl or ethyl; O(C₁-C₆)alkyl, preferably O-methyl,
A is C(O)NHOH,
L is defined by a covalent bond or —(CH₂)_{q}—, where
   q is 1 or 2;
R₄ is phenyl or pyridyl, substituted by a radical T-Z, where
   T is defined by a covalent bond or —O—, and
   Z is selected from the group of phenyl or pyridyl, where the phenyl or pyridyl group is unsubstituted or is substituted by 1, 2 or 3 substitutents, preferably one substitutent, and the substitutents are selected independently of one another from the group of F, Cl or Br, preferably Cl; O(C₁-C₆)alkyl, where one or more H atoms may be replaced by F atoms, preferably O-methyl, O-ethyl, OCF₃ or OCH₂CF₃; or NR₁₂R₁₃, where R₁₂ and R₁₃ are defined independently of one another by H, or C(O)—O—(C₁-C₆)alkyl,
n is 1,
   and the pharmacologically acceptable salts thereof.

The invention further relates particularly preferably to a compound of the formula (I) where
R₁, R₂ and R₃ are independently of one another H; F; NO₂; (C₁-C₆)alkyl, preferably methyl or ethyl; O(C₁-C₆)alkyl, preferably O-methyl,
A is C(O)NHOH,
L is defined by a covalent bond or —(CH₂)_{q}— where
   q is 1 or 2;
R₄ is pyridyl substituted by a radical T-Z where
   T is defined by a covalent bond or —O—, and
   Z is selected from the group of phenyl or pyridyl, where the phenyl or pyridyl group is unsubstituted or is substituted by 1, 2 or 3 substitutents, preferably one substitutent, and the substitutents are selected independently of one another from the group of F, Cl or Br, preferably Cl; O(C₁-C₆)alkyl, where one or more H atoms may be replaced by F atoms, preferably O-methyl, O-ethyl, OCF₃ or OCH₂CF₃; or NR₁₂R₁₃, where R₁₂ and R₁₃ are defined independently of one another by H, or C(O)—O—(C₁-C₆)alkyl,
n is 1,
   and the pharmacologically acceptable salts thereof.

Specifically preferred compounds of the formula (I) are selected from the group of
1. isopropyl[4′-(1-hydroxycarbamoyl-3,4-dihydro-1H-isoquinoline-2-sulfonyl)-biphenyl-4-yl]carbamate;
2. but-2-ynyl[4′-(1-hydroxycarbamoyl-3,4-dihydro-1H-isoquinoline-2-sulfonyl)-biphenyl-4-yl]carbamate;
3. 2-(6-phenoxypyridine-3-sulfonyl)-1,2,3,4-tetrahydroisoquinoline-1-hydroxycarboxamide;
4. 2-(6-morpholin-4-ylpyridine-3-sulfonyl)-1,2,3,4-tetrahydroisoquinoline-1-hydroxycarboxamide;
5. 2-(2-biphenyl-4-ylethanesulfonyl)-1,2,3,4-tetrahydroisoquinoline-1-hydroxycarboxamide;
6. 2-(4-pyrazol-1-ylbenzenesulfonyl)-1,2,3,4-tetrahydroisoquinoline-1-hydroxycarboxamide;
7. but-2-ynyl(R)-[4′-(1-hydroxycarbamoyl-3,4-dihydro-1H-isoquinoline-2-sulfonyl)-biphenyl-4-yl]carbamate;
8. but-2-ynyl(S)-[4′-(1-hydroxycarbamoyl-3,4-dihydro-1H-isoquinoline-2-sulfonyl)-biphenyl-4-yl]carbamate;
9. (R)-2-(4-pyrazol-1-ylbenzenesulfonyl)-1,2,3,4-tetrahydroisoquinoline-1-hydroxycarboxamide;
10. (S)-2-(4-pyrazol-1-ylbenzenesulfonyl)-1,2,3,4-tetrahydroisoquinoline-1-hydroxycarboxamide;
11. (R)-2-(6-phenoxypyridine-3-sulfonyl)-1,2,3,4-tetrahydroisoquinoline-1-hydroxycarboxamide;
12. (S)-2-(6-phenoxypyridine-3-sulfonyl)-1,2,3,4-tetrahydroisoquinoline-1-hydroxycarboxamide;
13. but-2-ynyl {4-[2-(1-hydroxycarbamoyl-3,4-dihydro-1H-isoquinoline-2-sulfonyl)-ethyl]phenyl}carbamate;
14. (R)-2-(2-biphenyl-4-ylethanesulfonyl)-1,2,3,4-tetrahydroisoquinoline-1-hydroxycarboxamide;
15. (S)-2-(2-biphenyl-4-ylethanesulfonyl)-1,2,3,4-tetrahydroisoquinoline-1-hydroxycarboxamide;
16. but-2-ynyl(R)-{4-[2-(1-hydroxycarbamoyl-3,4-dihydro-1H-isoquinoline-2-sulfonyl)ethyl]phenyl}carbamate;
17. but-2-ynyl(S)-{4-[2-(1-hydroxycarbamoyl-3,4-dihydro-1H-isoquinoline-2-sulfonyl)ethyl]phenyl}carbamate;
18. (R)-2-(6-morpholin-4-ylpyridine-3-sulfonyl)-1,2,3,4-tetrahydroisoquinoline-1-hydroxycarboxamide;
19. (S)-2-(6-morpholin-4-ylpyridin-3-sulfonyl)-1,2,3,4-tetrahydroisoquinoline-1-hydroxycarboxamide;
20. 2-{2-[4-(4-fluorophenoxy)phenyl]ethanesulfonyl}-1,2,3,4-tetrahydroisoquinoline-1-hydroxycarboxamide;
21. (R)-2-[4-(4-trifluoromethylphenoxy)benzenesulfonyl]-1,2,3,4-tetrahydro-isoquinoline-1-hydroxycarboxamide;
22. (R)-2-[4-(4-methoxyphenoxy)benzenesulfonyl]-1,2,3,4-tetrahydroisoquinoline-1-hydroxycarboxamide;
23. 2-[2-(4′-chlorobiphenyl-4-yl)ethanesulfonyl]-1,2,3,4-tetrahydroisoquinoline-1-hydroxycarboxamide;
24. (R)-2-[4-(4-chlorophenoxy)benzenesulfonyl]-1,2,3,4-tetrahydroisoquinoline-1-hydroxycarboxamide;
25. (R)-2-[4-(pyridin-2-yloxy)benzenesulfonyl]-1,2,3,4-tetrahydroisoquinoline-1-hydroxycarboxamide;
26. 2-[4-(pyridin-4-yloxy)benzenesulfonyl]-1,2,3,4-tetrahydroisoquinoline-1-hydroxycarboxamide;
27. 2-[6-(4-chlorophenoxy)pyridine-3-sulfonyl]-1,2,3,4-tetrahydroisoquinoline-1-hydroxycarboxamide;
28. 2-[2-(4′-chlorobiphenyl-4-yl)ethanesulfonyl]-6-nitro-1,2,3,4-tetrahydro-isoquinoline-1-hydroxycarboxamide;
29. (R)-2-[2-(4′-chlorobiphenyl-4-yl)ethanesulfonyl]-1,2,3,4-tetrahydroisoquinoline-1-hydroxycarboxamide;
30. (S)-2-[2-(4′-chlorobiphenyl-4-yl)ethanesulfonyl]-1,2,3,4-tetrahydroisoquinoline-1-hydroxycarboxamide;
31. (R)-2-[4-(4-methanesulfonylphenoxy)benzenesulfonyl]-1,2,3,4-tetrahydro-isoquinoline-1-hydroxycarboxamide;
32. (R)-2-[4-(4-methanesulfonylphenoxy)-2-methylbenzenesulfonyl]-1,2,3,4-tetrahydroisoquinoline-1-hydroxycarboxamide;
33. 2-(4′-chlorobiphenyl-4-sulfonyl)-6,7-dimethoxy-1,2,3,4-tetrahydroisoquinoline-1-hydroxycarboxamide;
34. (R)-2-(4′-chlorobiphenyl-4-sulfonyl)-1,2,3,4-tetrahydroisoquinoline-1-hydroxycarboxamide;
35. 2-[6-(4-cyanophenoxy)pyridine-3-sulfonyl]-1,2,3,4-tetrahydroisoquinoline-1-hydroxycarboxamide;
36. methyl {4-[5-(1-hydroxycarbamoyl-3,4-dihydro-1H-isoquinoline-2-sulfonyl)pyridine-2-yloxy]phenyl}acetate;
37. 2-[6-(3-acetylaminophenoxy)pyridine-3-sulfonyl]-1,2,3,4-tetrahydroisoquinoline-1-hydroxycarboxamide;
38. ethyl {4-[5-(1-hydroxycarbamoyl-3,4-dihydro-1H-isoquinoline-2-sulfonyl)pyridine-2-yloxy]phenyl}acetate;
39. 2-[6-(4-cyano-3-fluorophenoxy)pyridine-3-sulfonyl]-1,2,3,4-tetrahydro-isoquinoline-1-hydroxycarboxamide;
40. 2-[6-(1-oxoindan-5-yloxy)pyridine-3-sulfonyl]-1,2,3,4-tetrahydroisoquinoline-1-hydroxycarboxamide;
41. 2-[6-(2-methyl-1H-indol-5-yloxy)pyridine-3-sulfonyl]-1,2,3,4-tetrahydro-isoquinoline-1-hydroxycarboxamide;
42. (R)-2-[6-(4-methoxyphenoxy)pyridine-3-sulfonyl]-1,2,3,4-tetrahydroisoquinoline-1-hydroxycarboxamide;
43. (R)-2-[6-(4-trifluoromethoxyphenoxy)pyridine-3-sulfonyl]-1,2,3,4-tetrahydro-isoquinoline-1-hydroxycarboxamide;
44. (R)-2-{4-[4-(2,2,2-trifluoroethoxy)phenoxy]benzenesulfonyl}-1,2,3,4-tetrahydro-isoquinoline-1-hydroxycarboxamide;
45. (R)-2-[6-(4-chlorophenoxy)pyridine-3-sulfonyl]-1,2,3,4-tetrahydroisoquinoline-1-hydroxycarboxamide;
46. (R)-2-[6-(4-ethoxyphenoxy)pyridine-3-sulfonyl]-1,2,3,4-tetrahydroisoquinoline-1-hydroxycarboxamide;
47. (R)-2-(4-phenylsulfanylbenzenesulfonyl)-1,2,3,4-tetrahydroisoquinoline-1-hydroxycarboxamide;
48. (R)-4-(1-hydroxycarbamoyl-3,4-dihydro-1H-isoquinoline-2-sulfonyl)phenyl benzoate;
49. (R)-2-[4-(4-fluorobenzyloxy)benzenesulfonyl]-1,2,3,4-tetrahydroisoquinoline-1-hydroxycarboxamide;
50. (R)-2-[4′-(2,2,2-trifluoroethoxy)biphenyl-4-sulfonyl]-1,2,3,4-tetrahydro-isoquinoline-1-hydroxycarboxamide;
51. (R)-2-{6-[4-(2-methoxyethoxy)phenoxy]pyridine-3-sulfonyl}-1,2,3,4-tetrahydro-isoquinoline-1-hydroxycarboxamide;
52. (R)-2-[4-(4-hydroxyphenoxy)benzenesulfonyl]-1,2,3,4-tetrahydroisoquinoline-1-hydroxycarboxamide;
53. (S)-2-[6-(4-ethoxyphenoxy)pyridine-3-sulfonyl]-1,2,3,4-tetrahydroisoquinoline-1-hydroxycarboxamide;
54. 2-[4-(4-ethoxyphenoxy)-3-fluorobenzenesulfonyl]-1,2,3,4-tetrahydroisoquinoline-1-hydroxycarboxamide;
55. (R)-2-[4-(4-ethoxyphenoxy)-3-fluorobenzenesulfonyl]-1,2,3,4-tetrahydro-isoquinoline-1-hydroxycarboxamide;
56. 2-(6-butoxypyridine-3-sulfonyl)-1,2,3,4-tetrahydroisoquinoline-1-hydroxycarboxamide;
57. (R)-2-[4-(4-fluorophenoxy)benzenesulfonyl]-1,2,3,4-tetrahydroisoquinoline-1-hydroxycarboxamide;
58. (S)-2-[4-(4-fluorophenoxy)benzenesulfonyl]-1,2,3,4-tetrahydroisoquinoline-1-hydroxycarboxamide;
59. 2-[6-(4-trifluoromethoxyphenoxy)pyridine-3-sulfonyl]-1,2,3,4-tetrahydro-isoquinoline-1-hydroxycarboxamide;
60. 2-(6-dimethylaminopyridine-3-sulfonyl)-1,2,3,4-tetrahydroisoquinoline-1-hydroxycarboxamide;
61. 2-{3-fluoro-4-[4-(2-methoxyethoxy)phenoxy]benzenesulfonyl}-1,2,3,4-tetrahydroisoquinoline-1-hydroxycarboxamide;
62. 2-[3-chloro-4-(4-ethoxyphenoxy)benzenesulfonyl]-1,2,3,4-tetrahydroisoquinoline-1-hydroxycarboxamide;
63. 2-[4-(4-ethoxyphenoxy)benzenesulfonyl]-1,2,3,4-tetrahydroisoquinoline-1-hydroxycarboxamide;
64. (R)-2-[4-(4-trifluoromethoxyphenoxy)benzenesulfonyl]-1,2,3,4-tetrahydro-isoquinoline-1-hydroxycarboxamide;
65. (S)-2-[4-(4-trifluoromethoxyphenoxy)benzenesulfonyl]-1,2,3,4-tetrahydro-isoquinoline-1-hydroxycarboxamide;
66. (R)-2-[4-(4-ethoxyphenoxy)benzenesulfonyl]-1,2,3,4-tetrahydroisoquinoline-1-hydroxycarboxamide;
67. (S)-2-[4-(4-ethoxyphenoxy)benzenesulfonyl]-1,2,3,4-tetrahydroisoquinoline-1-hydroxycarboxamide;
68. 6-hydroxy-2-[4-(4-methoxyphenoxy)benzenesulfonyl]-1,2,3,4-tetrahydro-isoquinoline-1-hydroxycarboxamide;
69. (R)-2-{3-fluoro-4-[4-(2-methoxyethoxy)phenoxy]benzenesulfonyl}-1,2,3,4-tetrahydroisoquinoline-1-hydroxycarboxamide;
70. (S)-2-{3-fluoro-4-[4-(2-methoxyethoxy)phenoxy]benzenesulfonyl}-1,2,3,4-tetrahydroisoquinoline-1-hydroxycarboxamide.

The invention further relates to a compound of the formula (I) having formula (II) 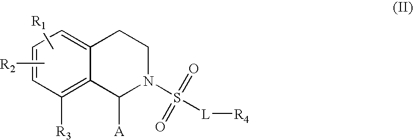
where R₁ and R₂ are independently of one another each H, F, Cl, Br, I, NO₂, CN, OH, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₃-C₈)cycloalkyl, O(C₁-C₄)alkyl-(C₃-C₈)cycloalkyl, O(C₃-C₈)cycloalkyl-(C₁-C₄)alkyl, OC(O)—(C₁-C₆)alkyl, OC(O)—(C₂-C₆)alkenyl, OC(O)—(C₃-C₈)cycloalkyl, OC(O)—(C₁-C₄)alkyl-(C₃-C₈)cycloalkyl, OC(O)—(C₃-C₈)cycloalkyl-(C₁-C₄)alkyl, C(O)O—(C₁-C₆)alkyl, C(O)O—(C₂-C₆)alkenyl, C(O)O—(C₃-C₈)cycloalkyl, C(O)O—(C₁-C₄)alkyl-(C₃-C₈)cycloalkyl, C(O)O—(C₃-C₈)cycloalkyl-(C₁-C₄)alkyl, C(O)NR₆R₇, NR₆R₇ or NR₆C(O)R₇, where
   R₆ and R₇ are independently of one another H or (C₁-C₆)alkyl,
R₃ is F, Cl, Br, I, NO₂, CN, OH, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₃-C₈)cycloalkyl, O(C₁-C₄)alkyl-(C₃-C₉)cycloalkyl, O(C₃-C₈)cycloalkyl-(C₁-C₄)alkyl, OC(O)—(C₁-C₆)alkyl, OC(O)—(C₂-C₆)alkenyl, OC(O)—(C₃-C₈)cycloalkyl, OC(O)—(C₁-C₄)alkyl-(C₃-C₈)cycloalkyl, OC(O)—(C₃-C₈)cycloalkyl-(C₁-C₄)alkyl, C(O)O—(C₁-C₆)alkyl, C(O)O—(C₂-C₆)alkenyl, C(O)O—(C₃-C₈)cycloalkyl, C(O)O—(C₁-C₄)alkyl-(C₃-C₈)cycloalkyl, C(O)O—(C₃-C₈)cycloalkyl-(C₁-C₄)alkyl, C(O)NR₆R₇, NR₆R₇ or NR₆C(O)R₇,
A is C(O)R₅ or CH₂SH, where
   R₅ is OR₆, NR₆R₇ or NR₆OH, where
n is 0, 1 or 2;
L is defined by —O—, —NR₁₄—, a covalent bond or —(CH₂)_{q}—, where
   R₁₄ is defined by H or (C₁-C₆)alkyl, and
   q is 1, 2, 3 or 4, and
R₄ is phenyl or (C₅-C₁₄)heteroaryl, where the phenyl or (C₅-C₁₄)heteroaryl radical is optionally substituted by 1, 2 or 3 substitutents independently of one another selected from the group of F, Cl, Br, I, CN, OH, NO₂, (C₁-C₆)alkyl, (C₂-C₆)alkenyl, (C₂-C₆)alkynyl, (C₃-C₈)cycloalkyl, (C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₂-C₆)alkynyl, O(C₃-C₈)cycloalkyl, O(C₁-C₄)alkyl-(C₃-C₈)cycloalkyl, O(C₃-C₈)cycloalkyl-(C₁-C₄)alkyl, —O(C₁-C₄)—O—(C₁-C₆) or a radical NR₈R₉, where
   R₈ and R₉ are defined independently of one another by H, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, (C₂-C₆)alkynyl, C(O)—V—(C₁-C₆)alkyl, C(O)—V—(C₂-C₆)alkenyl, C(O)—V—(C₃-C₈)cycloalkyl, C(O)—V—(C₁-C₄)alkyl-(C₃-C₈)cycloalkyl, C(O)—V—(C₃-C₈)cycloalkyl-(C₁-C₄)alkyl or C(O)—V—(C₂-C₆)alkynyl, where
      V is a covalent bond, —O— or —NH—, and where
      R₈ and R₉ may together form a 5- or 6-membered ring, and where the phenyl or (C₅-C₁₄)heteroaryl radical is optionally substituted by a group T-Z, where
   T is defined by a covalent bond, —O—, —S—, —O(C₁-C₄)alkyl-, —N(R₁₀)—, —C(O)—, —C(O)O—, —OC(O)—, —C(O)N(R₁₀)—, —N(R₁₀)—C(O)— or —N(R₁₀)—C(O)—N(R₁₁)—, where
      R₁₀ and R₁₁ are independently of one another H or (C₁-C₄)alkyl,
   Z is selected from the group of phenyl, (C₅-C₁₄)heteroaryl, (C₃-C₈)heterocycloalkyl or benzocyclo(C₅-C₇)alken-1-one,
   where phenyl, benzocyclo(C₅-C₇)alken-1-one, (C₅-C₁₄)heteroaryl or (C₃-C₈)heterocycloalkyl are unsubstituted or are substituted by 1, 2 or 3 substitutents independently of one another selected from the group of F, Cl, Br, I, CN, OH, NO₂, (C₁-C₆)alkyl, SO₂(C₁-C₆)alkyl, O(C₁-C₄)alkyl-O—(C₁-C₆)alkyl, —(C₁-C₄)alkyl-C(O)—O(C₁-C₆)alkyl, O(C₁-C₆)alkyl, (C₂-C₆)alkenyl, (C₃-C₈)cycloalkyl, —(C₁-C₄)alkyl-(C₃-C₈)cycloalkyl or —(C₃-C₈)cycloalkyl-(C₁-C₄)alkyl where one or more CH₂ groups in the alkenyl, alkyl or cycloalkyl radicals may be replaced by O or C(O), or else O(C₂-C₆)alkenyl, O(C₃-C₈)cycloalkyl, O(C₁-C₄)alkyl-(C₃-C₈)cycloalkyl, O—(C₃-C₈)cycloalkyl-(C₁-C₄)alkyl, (C₂-C₆)alkynyl, O(C₂-C₆)alkynyl, or NR₁₂R₁₃ where
      R₁₂ and R₁₃ are defined independently of one another by H, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, (C₂-C₆)alkynyl, C(O)—W—(C₁-C₆)alkyl, C(O)—W—(C₂-C₆)alkenyl, C(O)—W—(C₃-C₈)cycloalkyl, C(O)—W—(C₁-C₄)alkyl-(C₃-C₈)cycloalkyl, C(O)—W—(C₃-C₈)cycloalkyl-(C₁-C₄)alkyl, or C(O)—W—(C₂-C₆)alkynyl, where
      W is a covalent bond, —O— or —NH—;
         where one or more H atoms in (C₁-C₆)alkyl, (C₁-C₄)alkyl, (C₂-C₆)alkenyl, (C₃-C₈)cycloalkyl or (C₂-C₆)alkynyl radicals may optionally be replaced independently of one another by F atoms,
         and the pharmacologically acceptable salts thereof.

The invention preferably relates to a compound of the formula (II) where
R₁, R₂, R₃, A and L are as defined above,
n is 1, and
R₄ is a pyridyl radical, where the pyridyl radical is optionally substituted by 1, 2 or 3 substitutents independently of one another selected from the group of F, Cl, Br, I, CN, OH, NO₂, (C₁-C₆)alkyl, (C₂-C₆)alkenyl, (C₂-C₆)alkynyl, —(C₃-C₈)cycloalkyl, (C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₂-C₆)alkynyl, O(C₃-C₈)cycloalkyl, O(C₁-C₄)alkyl-(C₃-C₈)cycloalkyl, O(C₃-C₈)cycloalkyl-(C₁-C₄)alkyl, —O(C₁-C₄)alkyl-O—(C₁-C₆)alkyl or a radical NR₈R₉, where
   R₈ and R₉ are defined independently of one another by H, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, (C₂-C₆)alkynyl, C(O)—V—(C₁-C₆)alkyl, C(O)—V—(C₂-C₆)alkenyl, C(O)—V—(C₃-C₈)cycloalkyl, C(O)—V—(C₁-C₄)alkyl-(C₃-C₉)cycloalkyl, C(O)—V—(C₃-C₈)cycloalkyl-(C₁-C₄)alkyl or C(O)—V—(C₂-C₆)alkynyl, where
      V is a covalent bond, —O— or —NH—, and where
      R₈ and R₉ may together form a 5- or 6-membered ring,
   and where the pyridyl radical is optionally substituted by a group T-Z, where
   T is defined by a covalent bond, —O—, —S—, —O(C₁-C₄)alkyl-, —N(R₁₀)—, —C(O)—, —C(O)O—, —OC(O)—, —C(O)N(R₁₀)—, —N(R₁₀)—C(O)— or —N(R₁₀)—C(O)—N(R₁₁)—, where
      R₁₀ and R₁₁ are independently of one another H or (C₁-C₄)alkyl,
   Z is selected from the group of phenyl, (C₅-C₁₄)heteroaryl, (C₃-C₈)heterocycloalkyl or benzocyclo(C₅-C₇)alken-1-one,
   where phenyl, benzocyclo(C₅-C₇)alken-1-one, (C₅-C₁₄)heteroaryl or (C₃-C₈)heterocycloalkyl are unsubstituted or substituted by 1, 2 or 3 substitutents independently of one another selected from the group of F, Cl, Br, I, CN, OH, NO₂, (C₁-C₆)alkyl, SO₂(C₁-C₆)alkyl, O(C₁-C₄)alkyl-O—(C₁-C₆)alkyl, —(C₁-C₄)alkyl-C(O)—O(C₁-C₆)alkyl, O(C₁-C₆)alkyl, (C₂-C₆)alkenyl, (C₃-C₈)cycloalkyl, —(C₁-C₄)alkyl-(C₃-C₈)cycloalkyl or —(C₃-C₈)cycloalkyl-(C₁-C₄)alkyl where one or more CH₂ groups in the alkenyl, alkyl or cycloalkyl radicals may be replaced by O or C(O), or else O(C₂-C₆)alkenyl, O(C₃-C₉)cycloalkyl, O(C₁-C₄)alkyl-(C₃-C₈)cycloalkyl, O(C₃-C₉)cycloalkyl-(C₁-C₄)alkyl, (C₂-C₆)alkynyl, O(C₂-C₆)alkynyl, or NR₁₂R₁₃, where
      R₁₂ and R₁₃ are defined independently of one another by H, (C₁-C₆)alkyl, (C₂-C₆)alkenyl, (C₃-C₉)cycloalkyl, —(C₁-C₄)alkyl-(C₃-C₈)cycloalkyl, —(C₃-C₈)cycloalkyl-(C₁-C₄)alkyl, (C₂-C₆)alkynyl, C(O)—W—(C₁-C₆)alkyl, C(O)—W—(C₂-C₆)alkenyl, C(O)—W—(C₃-C₈)cycloalkyl, C(O)—W—(C₁-C₄)alkyl-(C₃-C₈)cycloalkyl, C(O)—W—(C₃-C₈)cycloalkyl-(C₁-C₄)alkyl, or C(O)—W—(C₂-C₆)alkynyl, where
      W is a covalent bond, —O— or —NH—;
         and pharmacologically acceptable salts thereof.

Preference is further given to compounds of the formula (II) where
R₁ and R₂ are independently of one another H, F, Cl, Br, NO₂, CN, OH, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₃-C₉)cycloalkyl, —O—(C₁-C₄)alkyl-(C₃-C₈)cycloalkyl or —O—(C₃-C₉)cycloalkyl-(C₁-C₄)alkyl,
R₃ is F, Cl, Br, NO₂, CN, OH, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₃-C₈)cycloalkyl, —O—(C₁-C₄)alkyl-(C₃-C₈)cycloalkyl or —O—(C₃-C₈)cycloalkyl-(C₁-C₄)alkyl,
R₅ is OH, NH₂ or NHOH,
L is defined by a covalent bond or —(CH₂)_{q}— where
   q is 1 or 2,
R₄ is phenyl or pyridyl, where phenyl and pyridyl are optionally substituted by 1, 2 or 3 substitutents independently of one another selected from the group of F, Cl, Br, CN, OH, NO₂, (C₁-C₆)alkyl, —O—(C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, O(C₂-C₆)alkenyl, O(C₃-C₈)cycloalkyl, —O—(C₁-C₄)alkyl-(C₃-C₈)cycloalkyl, —O—(C₃-C₈)cycloalkyl-(C₁-C₄)alkyl, (C₂-C₆)alkynyl, O(C₂-C₆)alkynyl, or NR₈R₉ where R₈ and R₉ are independently of one another H or (C₁-C₆)alkyl,
   and where phenyl or pyridyl is substituted by a group T-Z, where
   T is defined by a covalent bond or —O— and
   Z is selected from the group of phenyl, (C₅-C₁₀)heteroaryl, preferably pyridyl, pyrazolyl or indolyl, or C₅-C₇-heterocycloalkyl, particularly preferably morpholinyl, or benzocyclo(C₅-C₇)alken-1-on-yl, preferably indan-1-on-yl, where phenyl, (C₅-C₁₀)heteroaryl, C₅-C₇-heterocycloalkyl and benzocyclo(C₅-C₇)alken-1-on-yl are unsubstituted or are substituted by 1, 2 or 3 substitutents independently of one another selected from the group of F; Cl; Br; CN; OH; or
   (C₁-C₆)alkyl where one or more H atoms may be replaced by F atoms, preferably CF₃; —SO₂(C₁-C₆)alkyl, preferably —SO₂CH₃; or
   O(C₁-C₆)alkyl, where one or more H atoms may be replaced by F atoms, preferably OMe, OEt, O(CH₂)₃CH₃, OCF₃ or OCH₂CF₃;
   —CH₂—C(O)—O(C₁-C₆)alkyl, preferably —CH₂—C(O)—OMe or —CH₂—C(O)—OEt; —O—(C₁-C₄)alkyl-O—(C₁-C₆)alkyl, preferably —O(CH₂)₂OCH₃; NR₁₂R₁₃ where R₁₂ and R₁₃ are defined independently of one another by H, C(O)—W—(C₁-C₆)alkyl, or C(O)—W—(C₂-C₆)alkynyl, where W is a covalent bond or —O—, preferably NHC(O)O(i-Pr), NHC(O)OCH₂C≡CCH₃ or NHC(O)CH₃; and
n is 1,
   where one or more H atoms in (C₁-C₆)alkyl, (C₁-C₄)alkyl, (C₂-C₆)alkenyl, (C₃-C₈)cycloalkyl or (C₂-C₆)alkynyl radicals may optionally be replaced independently of one another by F atoms,
   and the pharmacologically acceptable salts thereof.

Particular preference is given to compounds of the formula (II) where
R₁ and R₂ are independently of one another H or (C₁-C₆)alkyl,
R₃ is F, Cl, Br, (C₁-C₆)alkyl or O(C₁-C₆)alkyl,
A is C(O)NHOH,
L is defined by a covalent bond or —(CH₂)_{q}— where
   q is 1 or 2,
R₄ are phenyl or preferably pyridyl, optionally substituted by 1, 2 or 3 radicals independently of one another selected from the group of F; Cl; NO₂; (C₁-C₆)alkyl, preferably methyl or ethyl; O(C₁-C₆)alkyl, preferably O-methyl; where phenyl and pyridyl are further substituted by a group T-Z, and
   T is defined by a covalent bond; —O—; —S—; —O—(C₁-C₄)alkyl-, preferably —O—CH₂—; or —O—C(O)—, and
   Z is selected from the group of phenyl or pyridyl,
      where phenyl or pyridyl are unsubstituted or substituted by 1, 2 or 3 substitutents independently of one another selected from the group of F; Cl; or O(C₁-C₆)alkyl, where one or more H atoms may be replaced by F atoms, preferably O-methyl, O-ethyl or OCF₃;
n is 1,
   and the pharmacologically acceptable salts thereof.

Particular preference is further given to compounds of the formula (II) where
R₁ and R₂ are independently of one another H, methyl or ethyl,
R₃ is F or O-methyl,
A is C(O)NHOH,
R₄ is phenyl or preferably pyridyl, substituted by a radical T-Z, where
   T is defined by a covalent bond or —O—, and
   Z is selected from the group of phenyl or pyridyl, where the phenyl or pyridyl group is unsubstituted or is substituted by 1, 2 or 3 substitutents, preferably one substitutent, and the substitutents are selected independently of one another from the group of F, Cl, Br, or O(C₁-C₆)alkyl, where one or more H atoms in (C₁-C₆)alkyl radicals may optionally be replaced independently of one another by F atoms; preferably F, Cl, O-methyl, O-ethyl, OCF₃,
L is defined by a covalent bond, and
n is 1,
   and the pharmacologically acceptable salts thereof.

Specifically particularly preferred compounds of the formula (II) are those selected from the group of
71. 8-fluoro-5-methyl-2-(6-phenoxypyridine-3-sulfonyl)-1,2,3,4-tetrahydro-isoquinoline-1-hydroxycarboxamide;
72. 2-(4′-chlorobiphenyl-4-sulfonyl)-5-fluoro-8-methoxy-1,2,3,4-tetrahydro-isoquinoline-1-hydroxycarboxamide;
73. 2-(4′-chlorobiphenyl-4-sulfonyl)-8-fluoro-5-methyl-1,2,3,4-tetrahydro-isoquinoline-1-hydroxycarboxamide;
74. 5-ethyl-8-fluoro-2-(6-phenoxypyridine-3-sulfonyl)-1,2,3,4-tetrahydro-isoquinoline-1-hydroxycarboxamide;
75. 2-(4′-chlorobiphenyl-4-sulfonyl)-5-ethyl-8-fluoro-1,2,3,4-tetrahydroisoquinoline-1-hydroxycarboxamide;
76. 8-fluoro-2-[6-(4-methoxyphenoxy)pyridine-3-sulfonyl]-5-methyl-1,2,3,4-tetrahydroisoquinoline-1-hydroxycarboxamide;
77. 8-fluoro-5-methyl-2-[6-(4-trifluoromethoxyphenoxy)pyridine-3-sulfonyl]-1,2,3,4-tetrahydroisoquinoline-1-hydroxycarboxamide;
78. 2-[4-(4-chlorophenoxy)benzenesulfonyl]-8-fluoro-5-methyl-1,2,3,4-tetrahydro-isoquinoline-1-hydroxycarboxamide;
79. 8-fluoro-5-methyl-2-[4-(pyridin-4-yloxy)benzenesulfonyl]-1,2,3,4-tetrahydro-isoquinoline-1-hydroxycarboxamide;
80. 8-fluoro-5-ethyl-2-[4-(pyridin-4-yloxy)benzenesulfonyl]-1,2,3,4-tetrahydro-isoquinoline-1-hydroxycarboxamide;
81. 2-[6-(4-ethoxyphenoxy)pyridine-3-sulfonyl]-8-fluoro-5-methyl-1,2,3,4-tetrahydroisoquinoline-1-hydroxycarboxamide;
82. 2-[4-(4-chlorophenoxy)benzenesulfonyl]-5-ethyl-8-fluoro-1,2,3,4-tetrahydro-isoquinoline-1-hydroxycarboxamide;
83. (R)-2-[4-(4-chlorophenoxy)benzenesulfonyl]-5-methyl-8-fluoro-1,2,3,4-tetrahydroisoquinoline-1-hydroxycarboxamide;
84. (S)-2-[4-(4-chlorophenoxy)benzenesulfonyl]-5-methyl-8-fluoro-1,2,3,4-tetrahydroisoquinoline-1-hydroxycarboxamide;
85. (R)-2-[6-(4-ethoxyphenoxy)pyridine-3-sulfonyl]-5-methyl-8-fluoro-1,2,3,4-tetrahydroisoquinoline-1-hydroxycarboxamide;
86. (S)-2-[6-(4-ethoxyphenoxy)pyridine-3-sulfonyl]-5-methyl-8-fluoro-1,2,3,4-tetrahydroisoquinoline-1-hydroxycarboxamide;
87. 5-methyl-2-[4-(4-fluorophenoxy)benzenesulfonyl]-8-fluoro-1,2,3,4-tetrahydro-isoquinoline-1-hydroxycarboxamide;
88. (R)-5-methyl-2-[4-(4-fluorophenoxy)benzenesulfonyl]-8-fluoro-1,2,3,4-tetrahydroisoquinoline-1-hydroxycarboxamide;
89. (S)-5-methyl-2-[4-(4-fluorophenoxy)benzenesulfonyl]-8-fluoro-1,2,3,4-tetrahydroisoquinoline-1-hydroxycarboxamide.

If the compounds of the formulae (I) or (II) contain one or more centers of asymmetry, they may have both the S and the R configuration independently of one another. The compounds may be in the form of pure optical isomers, of diastereomers, of racemates or of mixtures in all ratios.

### Definition of Terms

(C₅-C₁₄)-Heteroaryl radicals are aromatic mono-, bi- or tricyclic (C₅-C₁₄) ring compounds in which one or more ring atoms are oxygen atoms, sulfur atoms or nitrogen atoms, e.g. 1, 2, 3 or 4 nitrogen atoms, 1 or 2 oxygen atoms, 1 or 2 sulfur atoms or a combination of various heteroatoms. The heteroaryl radicals may be attached via all positions, for example via position 1, position 2, position 3, position 4, position 5, position 6, position 7 or position 8. Heteroaryl radicals may be unsubstituted or substituted one or more times, for example once, twice or three times, by identical or different radicals R₁.

Examples of appropriate heteroaryls are 2- or 3-thienyl, 2- or 3-furyl, 1-, 2- or 3-pyrrolyl, 1-, 2-, 4- or 5-imidazolyl, 1-, 3-, 4- or 5-pyrazolyl, 1,2,3-triazol-1-, -4- or 5-yl, 1,2,4-triazol-1-, -3- or -5-yl, 1- or 5-tetrazolyl, 2-, 4- or 5-oxazolyl, 3-, 4- or 5-isoxazolyl, 1,2,3-oxadiazol-4- or 5-yl, 1,2,4-oxadiazol-3- or -5-yl, 1,3,4-oxadiazol-2-yl or -5-yl, 2-, 4- or 5-thiazolyl, 3-, 4- or 5-isothiazolyl, 1,3,4-thiadiazol-2- or -5-yl, 1,2,4-thiadiazol-3- or -5-yl, 1,2,3-thiadiazol-4- or 5-yl, 2-, 3- or 4-pyridyl, 2-, 4-, 5- or 6-pyrimidinyl, 3- or 4-pyridazinyl, pyrazinyl, 1-, 2-, 3-, 4-, 5-, 6- or 7-indolyl, 1-, 2-, 4- or 5-benzimidazolyl, 1-, 3-, 4-, 5-, 6- or 7-indazolyl, 2-, 3-, 4-, 5-, 6-, 7- or 8-quinolyl, 1-, 3-, 4-, 5-, 6-, 7- or 8-isoquinolyl, 2-, 4-, 5-, 6-, 7- or 8-quinazolinyl, 3-, 4-, 5-, 6-, 7- or 8-cinnolinyl, 2-, 3-, 5-, 6-, 7- or 8-quinoxalinyl, 1-, 4-, 5-, 6-, 7- or 8-phthalazinyl. Also included are the corresponding N-oxides of these compounds, i.e. for example 1-oxy-2-, 3- or 4-pyridyl.

Preferred heteroaryl radicals are the 5- or 6-membered heteroaryl radicals, for example imidazolyl, pyrazolyl, pyrrolyl, triazolyl, tetrazolyl, thiazolyl and oxazolyl, and pyridyl and pyrimidinyl. Also preferred are the fused ring systems benzofuranyl, benzimidazolyl and indolyl. Pyrazolyl, indolyl and pyridyl is specifically preferred.

The term (CH₂)_{q} in which q is the integer zero, 1, 2, 3 or 4 means for example the methylene radical when n is 1 and the ethylene radical when n is 2. Appropriate CH₂ units are also the terminal CH₃ groups in an alkyl chain, which are regarded in this connection as CH₂—H groups. An analogous statement applies to CH units, which can be regarded both as tertiary carbons but also as part of a CH₂— (—HCH—)— or CH₃—(H₂CH—) group.

The term (C₁-C₆)-alkyl means hydrocarbon radicals whose carbon chain is straight-chain or branched and comprises 1 to 6 carbon atoms, for example methyl, ethyl, n-propyl, isopropyl, n-butyl, isobutyl, tertiary butyl, pentyl, isopentyl, neopentyl, hexyl, 2,3-dimethylbutane or neohexyl. The term —(C₁-C₄)-alkyl as subset of (C₁-C₆)-alkyl means hydrocarbon radicals whose carbon chain is straight-chain or branched and comprises 1 to 4 carbon atoms, for example methyl, ethyl, n-propyl, isopropyl, iso-butyl, butyl or tertiary butyl. A (C₁-C₆)alkyl group in which one or more H atoms are replaced by F atoms is, for example, trifluoromethyl, trifluoroethyl. An O(C₁-C₆)alkyl group for example methoxy, ethoxy. An O(C₁-C₆)alkyl in which one or more H atoms are replaced by F atoms is, for example, trifluoromethoxy or trifluoroethoxy.

The term (C₂-C₆)-alkenyl means hydrocarbon radicals whose carbon chain is straight-chain or branched and comprises 2 to 6 carbon atoms and have, depending on the chain length, 1, 2 or 3 double bonds, for example ethenylene, propenylene, isopropenylene, isobutenylene or butenylene; the substitutents on the double bond may where possible in principle have the E or Z orientation. The double bonds may be both internal and terminal.

The term (C₂-C₆)-alkynylene means hydrocarbon radicals whose carbon chain is straight-chain or branched and comprises 2 to 6 carbon atoms and have, depending on the chain length, 1 or 2 triple bonds, for example ethynyl, propenyl, isopropynyl, isobuthylynyl, butynyl, pentynyl or isomers of pentynyl or hexynyl or isomers of hexynyl. The triple bonds may be both internal and terminal.

The term (C₃-C₈)-cycloalkyl means radicals derived from 3- to 8-membered monocycles such as cyclopropyl, cyclobutyl, cyclopentyl, cyclohexyl, cycloheptyl or cyclooctyl. A —(C₁-C₄)alkyl-(C₃-C₈)cycloalkyl group is a terminal (C₃-C₈)cycloalkyl group which is linked via a (C₁-C₄)alkyl radical, for example cyclopropylmethyl.

The term (C₃-C₈)-heterocycloalkyl means radicals derived from 3- to 8-membered monocycles such as cyclopropyl, cyclobutyl, cyclopentyl, cyclohexyl, cycloheptyl or cyclooctyl, in which one or more ring atoms are oxygen atoms, sulfur atoms or nitrogen atoms, e.g. 1, 2, 3 or 4 nitrogen atoms, 1 or 2 oxygen atoms, 1 or 2 sulfur atoms or a combinations of various heteroatoms. The (C₃-C₈)-heterocycloalkyl radicals may be attached via all positions, for example via position 1, position 2, position 3, position 4, position 5, position 6, position 7 or position 8. (C₃-C₈)-Heterocycloalkyl radicals may be unsubstituted or substituted one or more times, for example once, twice or three times, by identical or different radicals R₁. (C₃-C₈)-Heterocycloalkyl radicals are, for example, pyrrolidinyl, tetrahydrothiophenyl, tetrahydrofuranyl, piperidinyl, pyranyl, dioxanyl, morpholinyl. (C₅-C₆)-Heterocycloalkyl radicals are preferred, and morpholinyl is particularly preferred.

Benzocyclo(C₅-C₇)alken-1-one radicals are radicals comprising a (C₅-C₇) ring fused to a benzyl ring and thus comprising 9-11 carbon atoms. 1,2-Benzo-1,2-(C₅-C₇)alken-one derivatives are preferred, and the (C₅-C₇) ring is particularly preferably perhydrogenated. Benzocyclo(C₅-C₇)alken-1-ones are, for example, indan-1-one; 3,4-dihydro-2H-naphthalen-1-one or 6,7,8,9-tetrahydrobenzocyclohepten-5-one, particularly preferably indan-1-one.

Unless mentioned otherwise, it is optionally possible for one or more H atoms in (C₁-C₆)alkyl, (C₁-C₄)alkyl, (C₂-C₆)alkenyl, (C₃-C₈)cycloalkyl or (C₂-C₆)alkynyl radicals to be replaced independently of one another by F atoms.

Pharmacologically acceptable salts of compounds of the formula (I) mean both their organic and inorganic salts as described in Remington's Pharmaceutical Sciences (17th edition, page 1418 (1985)). Because of the physical and chemical stability and the solubility, preference is given for acidic groups inter alia to sodium, potassium, calcium and ammonium salts; preference is given for basic groups inter alia to salts of maleic acid, fumaric acid, succinic acid, malic acid, tartaric acid, methylsulfonic acid, hydrochloric acid, sulfuric acid, phosphoric acid or of carboxylic acids or sulfonic acids, for example as hydrochlorides, hydrobromides, phosphates, sulfates, methanesulfonates, acetates, lactates, maleates, fumarates, malates, gluconates, and salts of amino acids, of natural bases or carboxylic acids. The preparation of physiologically tolerated salts from compounds of the formula (I) and (II) which are capable of salt formation, including their stereoisomeric forms, takes place in a manner known per se. The compounds of the formula (I) and (II) form stable alkali metal, alkaline earth metal or optionally substituted ammonium salts with basic reagents such as hydroxides, carbonates, bicarbonates, alcoholates and ammonia or organic bases, for example trimethyl- or triethylamine, ethanolamine, diethanolamine or triethanolamine, trometamol or else basic amino acids, for example lysine, ornithine or arginine. Where the compounds of the formula (I) or (II) have basic groups, stable acid addition salts can also be prepared with strong acids. Suitable for this purpose are both inorganic and organic acids such as hydrochloric, hydrobromic, sulfuric, hemisulfuric, phosphoric, methanesulfonic, benzenesulfonic, p-toluenesulfonic, 4-bromobenzenesulfonic, cyclohexylamidosulfonic, trifluoromethylsulfonic, 2-hydroxyethanesulfonic, acetic, oxalic, tartaric, succinic, glycerolphosphoric, lactic, malic, adipic, citric, fumaric, maleic, gluconic, glucuronic, palmitic, or trifluoroacetic acid.

The invention further relates to a process for preparing compounds of the formulae (I) and (II) which is characterized as follows.

Compounds of the formulae (I) which are unsubstituted in the basic tetrahydroisoquinoline structure can be prepared starting from commercially available tetrahydroisoquinoline-1-carboxylic acid (IV). As an alternative thereto, (IV) can be synthesized by catalytic hydrogenation of the commercially available isoquinoline-1-carboxylic acid (III) with hydrogen in the presence of PtO₂ (J. Chem. Soc. 1947, 129).

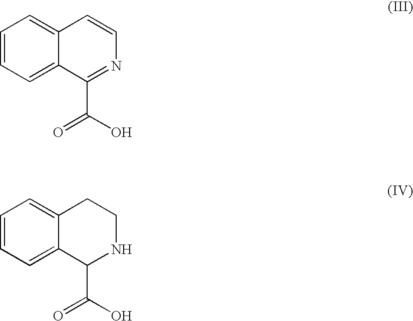

Other processes for the synthesis are likewise known. A broadly applicable example is Pictet-Spengler cyclization starting from 2-phenylethylamine. This process is described in detail in WO93/12091 where, in summary, 2-phenyl-1-aminoalkanes and aldehydes are condensed with addition of acid, for example HCl or trifluoroacetic acid.

Both synthetic possibilities afford in each case mixtures of enantiomers (racemates) of the compound of the formula (IV). Chiral tetrahydroisoquinoline-1-carboxylic acids are either commercially available or obtainable by described processes. It is possible for example to achieve separation into the optical antipodes via diastereomeric salts. A process with chiral 3-(4-nitrophenyl)-2-amino-1,3-propanediol is detailed for example in WO9312091 already mentioned. In this case, tetrahydroisoquinoline-1-carboxylic acid (IV) is converted into the appropriately N-protected benzyloxycarbonyl derivative which is then reacted by salt formation with the previously mentioned 3-(4-nitrophenyl)-2-amino-1,3-propanediol. The diastereomeric salts resulting therefrom can be separated from one another on the basis of different crystallization properties. Liberation of the acid and elimination of the urethane protective group finally affords both enantiomeric tetrahydroisoquinoline-1-carboxylic acids. It is likewise possible to carry out chromatographic separations on chiral phases with good results. For example, enantiopure chiral intermediates and final products can be obtained in this way, i.e. it is possible initially to synthesize a mixture of final products comprising the respective R- and S-tetrahydroisoquinoline-1-carboxylic acid derivatives, and subsequently to separate the latter for example by chiral HPLC methods.

The carboxylic acid (IV) can then be transformed by intermediate conversion into the corresponding trimethylsilyl ester and reaction with a sulfonyl chloride Cl—S(O)₂-L-R₄ into the sulfonamide (V). Silylating agents which can be used in this case are, for example, N,O-bistrimethylsilylacetamide (BSA) or N,O-bistrimethylsilyltrifluoracetamide.

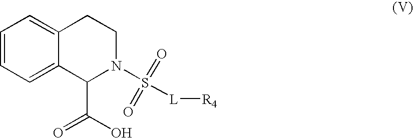

Sulfonamide (V) can then be converted into the analogous hydroxamic acid (VI). In this connection, the carboxylic acid is converted into the carbonyl chloride in a manner known to the skilled worker, such as, for example, by reaction with a chloroformic ester such as ethyl chloroformate ClC(O)OEt. Instead of the carbonyl chlorides it is possible to use the analogous mixed anhydrides. This is followed by reaction with hydroxylamine or an O-protected hydroxylamine, for example trimethylsilyl hydroxylamine, resulting in the desired hydroxamic acids after deprotection. For the example of trimethylsilyl-protected hydroxylamine, this takes place by acid workup.

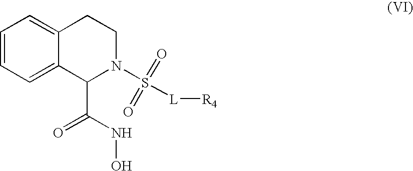

Synthesis of the substituted tetrahydroisoquinoline-1-hydroxamic acids is described below for the example of the 8-substituted derivatives and takes place for example by the following process:

Starting from a commercially available starting compounds of the formula (VII), the corresponding benzoyl carboxylic esters (VIII) are prepared by Friedel-Crafts acylation with, for example, Cl—C(O)—C(O)OEt in the presence of Lewis acid and/or protic acid (J. March, Advanced Organic Synthesis, 4^{th} Edition, John Wiley & Sons, 1992). Suitable Lewis acids are all conventional Lewis acids known to the skilled worker, such as, for example, AlCl₃, ZnCl₂, FeCl₃, TiCl₄, trifluoromethanesulfonates of the rare earth metals, for example scandium. An example of a protic acid which can be used is trifluoromethanesulfonic acid.

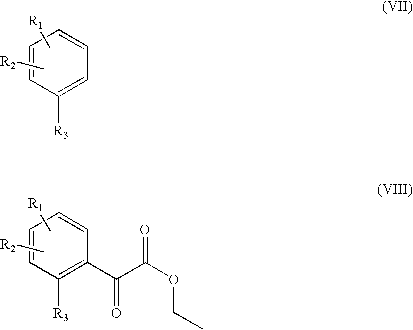

The Friedel-Crafts products (VIII) can then be converted by reductive amination with, for example, dimethoxyethylamine in a manner known to the skilled worker (see, for example, Roesky et al., Angewandte Chemie 2003, 42(24), 2708-2710) into an acetal of the formula (IX).

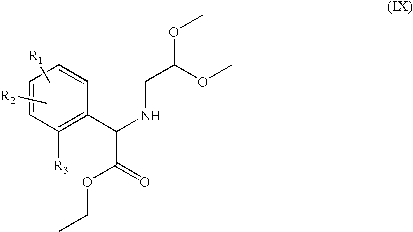

Subsequent reaction of the compound (IX) with acetyl chloride affords the acetamides (X) 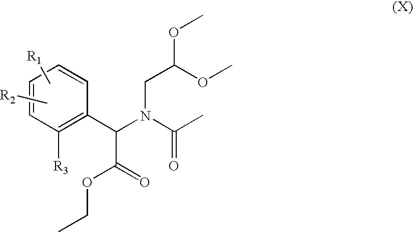
and a Lewis acid-mediated, preferably AlCl₃-mediated, cyclization results in dihydroisoquinolines of the formula (XI) (analogous to Journal of Organic Chemistry (1980), 45(10), 1950-1953).

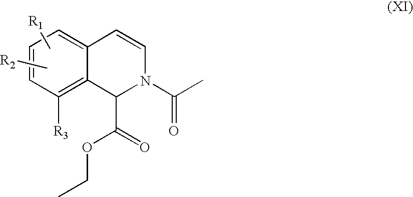

Catalytic hydrogenation with Pd/C and hydrogen affords a tetrahydroisoquinoline of the formula (XII) 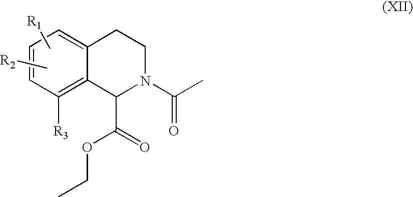
and a subsequent acidic amide and ester cleavage with, for example, concentrated hydrochloric acid results in the desired 8-substituted tetrahydroisoquinoline (XIII) in the form of the corresponding hydrochloride, which can be converted by treatment with equimolar amounts of base such as, for example, NaHCO₃ or organic amines such as, for example, triethylamine in a manner known to the skilled worker into the corresponding free base.

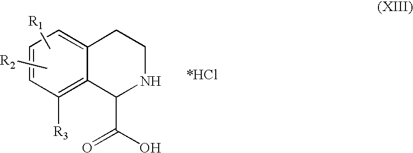

The further synthesis takes place in analogy to the reactions (IV)→(V)→(VI) shown above initially to give the carboxylic acids (XIV) starting from (XIII) using BSA and subsequent reaction with Cl—S(O)₂-L-R₄ and finally to give the desired hydroxamic acids (XV) using chloroformic esters, preferably ethyl chloroformate, subsequent reaction with a protected hydroxyamine and acidic workup:

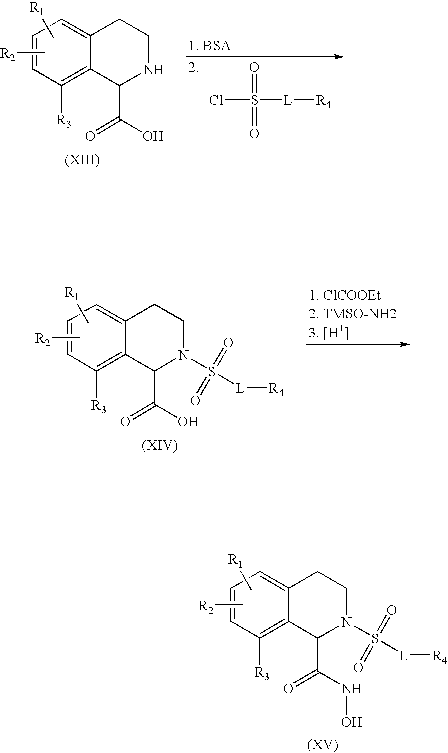

Alternatively, tetrahydroisoquinoline-1-carboxylic acids or esters thereof are possible via a Pictet-Spengler cyclization, where the corresponding phenylethylamines (XVI) is converted with a for example a glyoxylate, preferably ethyl glyoxylate into the desired tetrahydroisoquinoline-1-carboxylic ester of the formula (XVII).

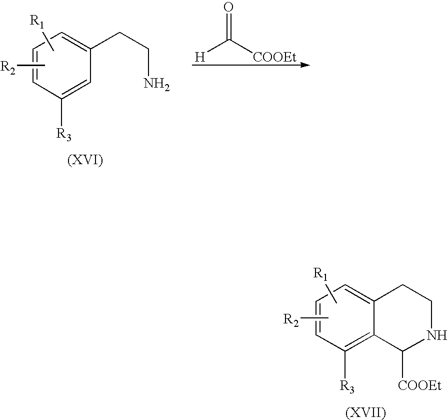

Obtaining the desired building blocks (XVII) enantiopure would be conceivable by using chiral esters of glyoxylic acid or analogs thereof (cf. Tetrahedron Lett; 40, 1999, 4969-4972). It is likewise possible to employ chiral Lewis acids in enantioselective Pictet-Spengler reactions.

A further alternative would start from the intermediates (XVI) to prepare the desired scaffold building blocks (XVII) by a Bischler-Napieralski reaction. For this purpose, the phenylethylamines (XVI) are converted in a manner known to the skilled worker into the corresponding glyoxylamides (XVIII), for example with ethyl glyoxalate, and these are then converted by treatment with POCl₃ and subsequent reduction, e.g. with complex hydrides or catalytic hydrogenation, into the desired building blocks XVII (cf. Org. Lett.; 6(16), 2931-2934, 2003).

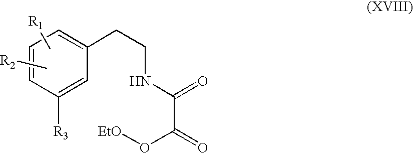

Obtaining the analogous 2,3,4,5-tetrahydro-1H-benzo[c]azepines starting from the phenylpropylamines which are homologous to (XVI) by means of a Pictet-Spengler reaction is described for example in J. Chem. Soc., Perkin. Trans. 1; 1974, 2602.

The radicals R₁, R₂, R₃, R₄ and L in the compounds of the formulae (V) to (XVIII) are (where present) defined in accordance with the general definition for the compound of the formula (I).

It has surprisingly been found that shifting the hydroxamic acid function from the 3 to the 1 position of the tetrahydroisoquinoline moiety makes it possible to increase considerably the metabolic stability of the compounds of the invention. Thus, compound (D) shows in vivo a considerably greater conversion into the carboxylic acid (E) than does the corresponding compound (F) into the carboxylic acid (G). This can be demonstrated unambiguously by means of the Cₘₐₓ ratios (hydroxamic acid:carboxylic acid).

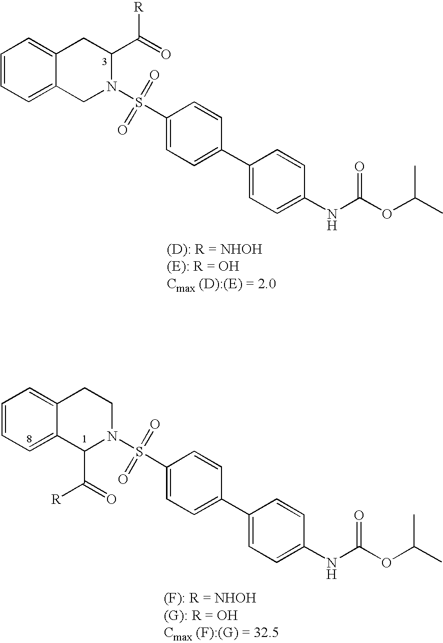

According to the present invention, a further stabilization of the hydroxamic acid functionality is achieved by introducing substitutents into position 8 of the tetrahydroisoquinoline moiety.

In addition, the compounds of the formula (I) show an increased selectivity in relation to MMP-2 and MMP-9 with slight inhibition of MMP-1. Adverse side effects are known from various clinical studies of MMP inhibitors, especially for the cancer indication. Several theories have been suggested to explain the mechanisms of the adverse side effects. Inter alia, MMP-1 inhibition has been suggested for musculoskeletal side effects (Heart Failure Reviews, 9, 63-79, 2004; Arthritis & Rheumatism, 48, 1742-1749, 2003).

For this reason, the compounds of the formula (I) or (II) which are preferred are those in which R₅ is defined by phenyl or heteroaryl substituted by 1, 2 or 3 substitutents, one of these substitutents being given by T-Z.

In addition, compounds of the formula (I) or (II) having a long side chain -L-R₄ show high selectivity in relation to MMP-1. This may be demonstrated by the following exemplary compounds:

For a comparison compound corresponding to Ma et al. (Bioorg. Med. Chem. Lett. 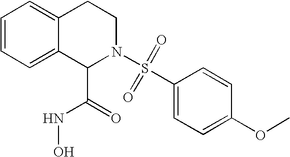
the ratio of MMP1 to MMP9 inhibition is 3.7.

For a compound of the formula (I) where
n is 1; R₁, R₂, R₃ is H; A is C(O)NHOH; L is a covalent bond; R₄ is phenyl substituted by a substitutent T-Z, where T is —O— and Z is phenyl substituted by a 4-methoxy substitutent, 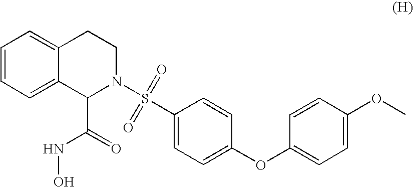
the ratio of MMP1 to MMP9 inhibition is 60.

The invention also relates to medicaments which have an effective content of at least one compound of the formula (I) and/or (II) and/or of a physiologically tolerated salt of the compound of the formula (I) and/or (II) and/or an optionally stereoisomeric form of the compound of the formula (I) and/or (II), together with a pharmaceutically suitable and physiologically tolerated carrier, additive and/or other active ingredients and excipients.

Because of the pharmacological properties, the compounds of the invention are suitable for the selective prophylaxis and/or therapy of all disorders in the progression of which an enhanced activity of metalloproteinases are involved. These include the indications described in the introduction. These are in particular cardiovascular disorders such as remodeling of the heart after a myocardial infarction and atherosclerosis. They further include unstable angina pectoris, heart failure, stenosis, septic shock and the prophylaxis of myocardial and cerebral infarctions. The compounds of the formula (I) and/or (II) are further suitable for the treatment of inflammations, cancers, tumor metastasis, cachexia, anorexia, ulceration, degenerative joint disorders such as osteoarthroses, spondyloses, chondrolysis following joint trauma or prolonged joint immobilization after meniscus or patella injuries or ligament tears. They also include connective tissue disorders such as collagenoses, periodontal disorders, wound-healing disturbances and chronic disorders of the locomotor system such as inflammatory, immunologically or metabolically related acute and chronic arthritides, arthropathies, myalgias and disturbances of bone metabolism.

The medicaments of the invention can be administered by oral, inhalational, rectal or transdermal administration or by subcutaneous, intraarticular, intraperitoneal or intravenous injection. Oral administration is preferred.

The invention also relates to a process for producing a medicament which comprises converting at least one compound of the formula (I) and/or (II) with a pharmaceutically suitable and physiologically tolerated carrier and, where appropriate, further suitable active ingredients, additives or excipients into a suitable dosage form.

Examples of suitable solid or pharmaceutical formulations are granules, powders, coated tablets, tablets, (micro)capsules, suppositories, syrups, oral solutions, suspensions, emulsions, drops or injectable solutions, and products with protracted release of active ingredient, in the production of which conventional physiologically tolerated excipients or carriers such as disintegrants, binders, coating agents, swelling agents, glidants or lubricants, flavorings, sweeteners and solubilizers are used. Excipients which are frequently used and which may be mentioned are magnesium carbonate, titanium dioxide, lactose, mannitol and other sugars, talc, milk protein, gelatin, starch, cellulose and its derivatives, animal and vegetable oils such as fish liver oil, sunflower, peanut or sesame oil, polyethylene glycol and solvents such as, for example, sterile water and monohydric or polyhydric alcohols such as glycerol.

The pharmaceutical products are preferably produced and administered in dosage units, each unit comprising as active ingredient a particular dose of the compound of the invention of the formula I. In the case of solid dosage units such as tablets, capsules, coated tablets or suppositories, this dose can be up to about 1000 mg, but preferably about 50 to 300 mg, and in the case of solutions for injection in ampoule form up to about 300 mg, but preferably about 10 to 100 mg.

The daily doses indicated for the treatment of an adult patient weighing about 70 kg are from about 2 mg to 1000 mg of active ingredient, preferably about 50 mg to 500 mg, depending on the activity of the compound of the formula (I) and/or (II). However, in some circumstances, higher or lower daily doses may also be appropriate. The daily dose may be administered both by administration once a day in the form of a single dosage unit or else a plurality of smaller dosage units, and by administration more than once a day in divided doses at defined intervals. The medicaments of the invention are generally administered orally or parenterally, but rectal use is also possible in principle. Examples of suitable solid or liquid pharmaceutical preparations are granules, powders, tablets, coated tablets, (micro)capsules, suppositories, syrups, emulsions, suspensions, aerosols, drops or injectable solutions in ampoule form, and products which with protracted release of active ingredient, in the production of which normally carriers and additions and/or aids such as disintegrants, binders, coating agents, swelling agents, glidants or lubricants, flavorings, sweeteners or solubilizers are used.

Examples of conventional pharmacologically suitable carriers or excipients are magnesium carbonate, titanium dioxide, lactose, mannitol and other sugars, talc, milk protein, gelatin, starch, vitamins, cellulose and its derivatives, animal or vegetable oils, polyethylene glycols and solvents such as, for example, sterile water, alcohol, glycerol and polyhydric alcohols.

The dosage units for oral administration may where appropriate be microencapsulated in order to delay delivery or extend it over a longer period, such as, for example, by coating or embedding the active ingredient in particulate form in suitable polymers, waxes or the like.

The pharmaceutical products are preferably produced and administered in dosage units, each unit comprising as active ingredient a particular dose of one or more compounds of the spirobenzofuran lactam derivatives of the invention. In the case of solid dosage units such as tablets, capsules and suppositories, this dose can be up to about 500 mg, but preferably about 0.1 to 200 mg, and in the case of solutions for injection in ampoule form up to about 200 mg, but preferably about 0.5 to 100 mg, per day. The daily dose to be administered depends on the body weight, age, gender and condition of the mammal. However, in some circumstances higher or lower daily doses may also be appropriate. The daily dose may be administered both by administration once a day in the form of a single dosage unit or else in a plurality of smaller dosage units, and by administration more than once a day in divided doses at defined intervals.

The medicaments of the invention are produced by converting one or more of the compounds of the invention of the formula (I) and/or (II) optionally with one or more of the conventional carriers or excipients and into a suitable dosage form.

The invention is explained further in the examples which follow. Percentage data relate to weight. Mixing ratios in the case of liquids relate to volume unless other statements have been made.

### EXAMPLES

List of Abbreviations Used:

abs. absolute
ACN acetonitrile
APMA p-aminophenylmercuric acetate
ar aryl (chemical shifts to be assigned to aryl protons)
BSA N,O-bistrimethylsilylacetamide
BSTFA bis(trimethylsilyl)trifluoracetamide
comp. compound
conc. concentrated
DMAP N,N-dimethylaminopyridine
DMF dimethylformamide
eq. equivalent
ex. example
FA formic acid
GM general method
LCMS liquid chromatography mass spectrometry
Merck Merck KGaA, Germany
m.p. melting point
Rₜ retention time
TFA trifluoroacetic acid
TLC thin layer chromatogram
YMC the YMC company, Japan
   Synthesis of the Tetrahydroisoquinoline Scaffolds:

### Example 1

### Scaffold A (8-fluoro-5-methyltetrahydroisoquinoline-1-carboxylic acid)

### Example 1.1

### Ethyl 2-fluoro-5-methylphenyl)oxoacetate

13.64 g (99.9 mmol) of ethyl oxalyl chloride were added to a suspension of 14.5 g (109.0 mmol) of AlCl₃ in 50 ml of dichloromethane at 0° C. and stirred at 0° C. for 30 minutes. After stirring at room temperature for a further 30 minutes, 10 g (90.8 mmol) of 4-fluorotoluene were added dropwise, and the mixture was stirred at room temperature for two hours. For workup, the reaction solution was poured onto ice, the organic phase was separated off, and the aqueous was extracted once with dichloromethane. The combined organic phases were dried with MgSO₄ and concentrated. After final purification of silica gel it was possible to obtain 7.39 g of the desired Friedel-Crafts product. Yield 39%.

### Example 1.2

### Ethyl 2,2-dimethoxyethylamino)(2-fluoro-5-methylphenyl)acetate

6.83 g (32.5 mmol) of ethyl (2-fluoro-5-methylphenyl)oxoacetate (from Example 1.1) were dissolved in 75 ml of abs. ethanol and, at room temperature, a solution of 17.08 g (162 mmol) of aminoacetaldehyde dimethyl acetal in 40 ml of abs. ethanol, and 7.80 g (130 mmol) of acetic acid were added. After one hour, 2.04 g (32.5 mmol) of sodium cyanoborohydride were added and stirring was continued at room temperature. After standing overnight, 25-30 ml of a sat. NaHCO₃ solution were added, and the reaction solution was concentrated in vacuo. The residue was taken up in H₂O and extracted three times with ethyl acetate. The combined organic phases were dried with MgSO₄ and the solvent was removed in vacuo. Purification on silica gel affords the title compound in a yield of 53%.

### Example 1.3

### Ethyl [acetyl(2,2-dimethoxyethyl)amino](2-fluoro-5-methylphenyl)-acetate

A spatula tip of DMAP was added to a solution of 1.0 g (3.17 mmol) of ethyl (2,2-dimethoxyethylamino)(2-fluoro-5-methylphenyl)acetate (from Example 1.2) in 10 ml of pyridine and, at 0° C., a solution of 498 mg (6.34 mmol) of acetyl chloride in 2 ml of dichloromethane was added dropwise. After one hour, the ice bath was removed and the mixture was stirred at room temperature for a further hour, after which it was possible to establish complete conversion. For workup, the mixture was diluted with dichloromethane and washed with sat. NaHCO₃ solution. The phases were separated and the aqueous was extracted once more with dichloromethane. The organic phases were washed twice with 2 N HCl and with H₂O. After drying with MgSO₄ and removal of the solvent in vacuo, the desired acetamide was obtained in quantitative yield, no further purification being necessary.

### Example 1.4

### Ethyl 2-acetyl-5-methyl-8-fluoro-1,2-dihydroisoquinoline-1-carboxylate

10.4 g (78.0 mmol) of AlCl₃ were dissolved in 200 ml of dichloroethane and, at room temperature, a solution of 3.8 g (11.13 mmol) of ethyl [acetyl-(2,2-dimethoxyethyl)-amino](2-fluoro-5-methylphenyl)acetate (from Example 1.3) in 90 ml of dichloroethane was added. The mixture was stirred at room temperature for two hours. After standing overnight, it was poured onto ice, and the organic phase was separated off. The aqueous phase was extracted twice more with dichloromethane, and the organic phases were dried with MgSO₄ and freed of solvent in vacuo. The crude product (3.64 g) obtained in this way can be reacted further without further purification.

### Example 1.5

### Ethyl 2-acetyl-5-methyl-8-fluoro-1,2,3,4-tetrahydroisoquinoline-1-carboxylate

3.64 g of ethyl 2-acetyl-5-methyl-8-fluoro-1,2-dihydroisoquinoline-1-carboxylate (from Example 1.4, crude product) were hydrogenated under standard conditions in 100 ml of ethanol with catalytic amounts of palladium on carbon (10%), with additional catalyst being added three times in order to achieve complete conversion. Filtration and removal of the solvent in vacuo were followed by purification on silica gel (dichloromethane/methanol 98:2), resulting in 2.62 g of the title compound. Yield 84% (two stages).

### Example 1.6

### Scaffold A (5-Methyl-8-fluorotetrahydroisoquinoline-1-carboxylic acid hydrochloride)

1.2 g (4.30 mmol) of ethyl 2-acetyl-5-methyl-8-fluoro-1,2,3,4-tetrahydroisoquinoline-1-carboxylate (from Example 1.5) were heated to reflux in 25 ml of conc. HCl for 2.5 hours. The solvent was then removed in vacuo, and the residue was taken up in H₂O and freeze-dried, resulting in 850 mg of the desired amino acid in the form of the corresponding hydrochloride. Yield 81%.

### Example 2

### Scaffold B (5-Ethyl-8-fluorotetrahydroisoquinoline-1-carboxylic acid)

### Example 2.1

### Ethyl 5-ethyl-2-fluorophenyl)oxoacetate

The preparation was carried out in analogy to Example 1.1. Yield: 57%.

### Example 2.2

### Ethyl 2,2-dimethoxyethylamino)(5-ethyl-2-fluorophenyl)acetate

The preparation was carried out in analogy to Example 1.2. Yield: 32%.

### Example 2.3

### Ethyl [acetyl(2,2-dimethoxyethyl)amino](5-ethyl-2-fluorophenyl)acetate

The preparation was carried out in analogy to Example 1.3. Yield: 77% after chromatography on silica gel (ethyl acetate/heptane 2:1).

### Example 2.4

### Ethyl 2-acetyl-5-ethyl-8-fluoro-1,2-dihydroisoquinoline-1-carboxylate

The preparation was carried out in analogy to Example 1.4. Yield: 54% after chromatography on silica gel (ethyl acetate/heptane 1:1).

### Example 2.5

### Ethyl 2-acetyl-5-ethyl-8-fluoro-1,2,3,4-tetrahydroisoquinoline-1-carboxylate

The preparation was carried out in analogy to Example 1.5. Yield: 70%.

### Example 2.6

### Scaffold B (5-Ethyl-8-fluorotetrahydroisoquinoline-1-carboxylic acid)

The preparation was carried out in analogy to Example 1.6. Yield: quantitative.

### Example 3

### Scaffold C (8-Methoxy-5-fluorotetrahydroisoquinoline-1-carboxylic acid)

### Example 3.1

### Ethyl 5-fluoro-2-methoxyphenyl)oxoacetate

The preparation was carried out in analogy to Example 1.1. Yield: 87%.

### Example 3.2

### Ethyl (2,2-dimethoxyethylamino)(5-fluoro-2-methoxyphenyl)acetate

The preparation was carried out in analogy to Example 1.2. Yield: 59%.

### Example 3.3

### Ethyl [acetyl-(2,2-dimethoxyethyl)amino](5-fluoro-2-methoxyphenyl)-acetate

The preparation was carried out in analogy to Example 1.3. Yield: 98% after chromatography on silica gel (ethyl acetate/heptane 2:1).

### Example 3.4

### Ethyl 2-acetyl-5-fluoro-8-hydroxy-1,2-dihydroisoquinoline-1-carboxylate

The preparation was carried out in analogy to Example 1.4. Yield: 73% after purification on silica gel (ethyl acetate/heptane 1:1).

### Example 3.5

### Ethyl 2-acetyl-5-fluoro-8-methoxy-1,2-dihydroisoquinoline-1-carboxylate

1.03 g (3.69 mmol) of ethyl 2-acetyl-5-fluoro-8-hydroxy-1,2-dihydroisoquinoline-1-carboxylate (from Example 3.4) were dissolved in 20 ml of abs. DMF, and 2.32 g (18.44 mmol) of dimethyl sulfate were added. At room temperature, 295 mg (7.38 mmol) of NaH (60%) were added, and the mixture was stirred at room temperature for two hours. For workup, the solvent was removed in vacuo, and the residue was dissolved in dichloromethane and washed with 1 N NaOH. The phases were separated, and the aqueous was extracted once more with dichloromethane. The combined organic phases were washed once more with 1 N NaOH and twice with H₂O, dried with MgSO₄ and concentrated. Purification on silica gel (ethyl acetate/heptane 1:2) affords the desired methyl ether in a yield of 50%.

### Example 3.6

### Ethyl 2-acetyl-5-fluoro-8-methoxy-1,2,3,4-tetrahydroisoquinoline-1-carboxylate

The preparation was carried out in analogy to Example 1.5. Yield: 81%.

### Example 3.7

### Scaffold C (8-Methoxy-5-fluorotetrahydroisoquinoline-1-carboxylic acid)

The preparation was carried out in analogy to Example 1.6. Yield: 96%.

### Example 4

### Scaffold D (tetrahydroisoquinoline-1-carboxylic acid)

Scaffold D was prepared by a process disclosed in the literature by catalytic hydrogenation (Adam's catalyst) from isoquinoline-1-carboxylic acid (J. Chem. Soc.; 1947, 129).

### Example 5

### Scaffold D1 (D-tetrahydroisoquinoline-1-carboxylic acid)

The enantiopure scaffold D1 is prepared as described in WO9312091 (vide supra) by separating the diastereomers by reaction with 3-(4-nitrophenyl)-2-amino-1,3-propanediol by methods known per se.

### Example 6

### Scaffold D2 (L-tetrahydroisoquinoline-1-carboxylic acid)

The enantiopure scaffold D2 is prepared as described in WO9312091 (vide supra) by separating the diastereomers by reaction with 3-(4-nitrophenyl)-2-amino-1,3-propanediol by methods known per se.

### Example 7

### Scaffold E (6-nitrotetrahydroisoquinoline-1-carboxylic acid)

Synthesis of the nitrated compound scaffold E can be carried out in analogy to the described synthesis of the nitrated tetrahydroisoquinoline-3-carboxylic acid derivatives which is described in U.S. Pat. No. 5,962,471. For this purpose, tetrahydroisoquinoline-1-carboxylic acid is reacted with potassium nitrate in conc. sulfuric acid while cooling, resulting in a mixture of 6- and 7-nitro-1,2,3,4-tetrahydroisoquinoline-1-carboxylic acid, which can preferably be separated by chromatographic methods.

### Example 8

### Scaffold F (6,7-dimethoxytetrahydroisoquinoline-1-carboxylic acid)

Scaffold F is commercially available. Alternatively, the scaffold can be prepared by known methods, for example by a Pictet-Spengler cyclization as described in J. Org. Chem. 1975, 40, 740-43.

Synthesis of the Sulfonyl Chlorides:

### Example 9

### 4-Chlorobiphenylethanesulfonyl chloride

### Example 9.1

### 1-(2-Bromoethanone)-4-(4-chlorophenyl)benzene

4-Chlorobiphenyl (23.6 g; 0.125 mol) was introduced in portions to a stirred suspension of AlCl₃ (34.7 g; 0.26 mol) and bromoacetyl bromide (25.2 g; 0.125 mol) in 400 ml of CS₂ at 0° C. and then heated under reflux for 3 h. The reaction mixture was subsequently poured slowly into ice and extracted with ethyl acetate, and the organic phase washed with aqueous NaHCO₃ solution and water. It was then dried over anhydrous sodium sulfate and evaporated under reduced pressure. The remaining residue was recrystallized from dichloromethane. Yield: 24.2 g (62% of theory). m.p: 127-128° C., ¹H-NMR(300 MHz): 5.0 (s, 2H, CH₂); 7.5-8.1 (4 d, 8H, ar); MS(M+H): 311.1.

### Example 9.2

### 4-Chlorobiphenylethane bromide

tert-Butylamine borane (27.5 g; 0.31 mol) was added to a stirred suspension of AlCl₃ (20.0 g; 0.15 mol) in dichloromethane (500 ml) at 0° C. After the mixture had stirred at 0° C. for 15 min, a solution of 1-(2-bromoethanone)-4-(4-chlorophenyl)benzene (from Example 9.1) (16.0 g; 50 mmol) in dichloromethane (150 ml) was added, and the mixture was stirred at 0° C. for a further 4 h. Cold dilute HCl (1N, 30 ml) was added dropwise, and then several extractions with ethyl acetate were carried out. The combined organic phases were washed first with dilute HCl and then with saturated brine and evaporated. An oily compound was obtained and was purified by flash chromatography on silica gel. Yield: 15 g (quantitative). m.p: 142° C.; ¹H-NMR(300 MHz): 3.2; 3.78 (2 t, 4H, CH₂); 7.4-7.7 (4 d, 8H, ar); MS(M+H): 296.2.

### Example 9.3

### Sodium salt of 4-chlorobiphenylethanesulfonic acid

4-Chlorobiphenylethane bromide (from Example 9.2) (14.8 g; 50 mmol) was dissolved in a mixture of ethanol and water (1:1, 200 ml). Sodium sulfite (9.5 g; 75 mmol) and tetrabutylammonium iodide (1.8 g, 5 mmol) were added, and the mixture was heated under reflux for 16 h. The reaction mixture was then decanted off from a small amount of a solid, and the volume was reduced by partial evaporation under reduced pressure. The product crystallized on cooling and was filtered off and recrystallized from MeOH/H₂O. It was then dried under reduced pressure. Yield: 13.9 g (94% of theory). ¹H-NMR(300 MHz): 2.6; 2.95 (2 m, 4H, CH₂); 7.3-7.7 (4 d, 8H, ar).

### Example 9.4

### 4-Chlorobiphenylethanesulfonyl chloride, 4-fluorobiphenylethane-sulfonyl chloride and biphenylethanesulfonyl chloride

Phosphorus pentachloride (3.2 g; 15 mmol) was added to a suspension of the sodium salt of 4-chlorobiphenylethanesulfonic acid (4.8 g; 15 mmol) in phosphorus oxychloride (50 ml). The mixture was heated at 60° C. for 6 h and then poured onto ice after addition of methylene chloride. The mixture was neutralized with saturated NaHCO₃ solution, and the organic phase was separated off, dried and evaporated under reduced pressure. Yield: 5 g (quantitative); ¹H-NMR(300 MHz): 2.9 (m, 4H, CH₂); 7.3-7.7 (4 d, 8H, ar).

4-Fluorobiphenylethanesulfonyl chloride and biphenylethanesulfonyl chloride can be prepared by the same method in analogy to 4-chlorobiphenylsulfonyl chloride. The sulfonyl chlorides used for the compounds of Examples 31 and 32 (Table 1) were prepared by known methods, see, for example, U.S. Pat. No. 4,349,568.

The sulfonyl chloride employed for the compound of Example 44 (Table 1) was prepared according to known examples from phenoxyphenol by reaction with the triflate of trifluoroethanol in analogy to US 20020103242 and subsequent chlorosulfonation with (1) chlorosulfonic acid and (2) oxalyl chloride as described in U.S. Pat. No. 6,153,757.

The acid chlorides employed for the compounds of Examples 47, 48, 49, 57 and 58 (Table 1) were prepared by chlorosulfonation/chlorination of the appropriate precursors as described in U.S. Pat. No. 6,153,757.

The sulfonyl chloride employed for the compound of Example 50 (Table 1) was prepared according to known examples starting from 4-phenylphenol by reaction with the triflate of trifluoroethanol in analogy to US 20020103242 and subsequent chlorosulfonation with (1) chlorosulfonic acid and (2) oxalyl chloride as described for example in U.S. Pat. No. 6,153,757.

### General Methods A1-A3: Synthesis of the Compounds of the Examples by Reaction of the Scaffolds A, B, C, D, D1, D2, E or F to Give the Corresponding N-sulfonyl-tetrahydroisoquinoline-1-carboxylic Acids

General Method A1

The tetrahydroisoquinoline-1-carboxylic acid building block (1.0 eq. of the particular scaffold) is introduced into dichloromethane (5 ml/1 mmol), and 2.0 eq. of diisopropylethylamine are added. Addition of 1.2 eq. of BSA is followed by stirring at room temperature for two hours and then, at 0° C., a solution of 1.2 eq. of the sulfonyl chloride in 5 ml of dichloromethane is added dropwise. After the reaction solution has stood at room temperature overnight it is washed with 1 N HCl. The phases are separated and the aqueous is extracted once more with dichloromethane. The combined organic phases are washed with H₂O, dried with MgSO₄ and freed of solvent in vacuo. Subsequent chromatography on silica gel affords the desired N-sulfonyltetrahydroisoquinoline-1-carboxylic acids.

General Method A2

A carboxylic acid was dissolved in 0.5-2 molar NaOH, where appropriate with the addition of 10-50% of an organic cosolvent tetrahydrofuran (THF) or DMF. The acid chloride (1-1.2 equivalents, preferably 1.1) was dissolved in THF (concentration 0.05 to 1 M) and slowly added dropwise. 2 N NaOH was automatically added in an autotitrator at room temperature to keep the pH constant. Adjusted pH: 8-12, preferably 9-11. After the reaction was complete, evident from no further NaOH consumption, the organic cosolvent was removed in a rotary evaporator, and the aqueous solution or suspension was mixed with ethyl acetate and acidified with 1 N HCl. After removal of the organic phase and renewed extraction of the aqueous phase with ethyl acetate, the organic phases were combined and dried over Na₂SO₄ and then the solvent was removed under reduced pressure. The crude product was either directly reacted further or purified by chromatography.

General Method A3

8 mmol of an imino acid were dissolved or suspended in 30 ml of acetonitrile. 2.3 g (9 mmol) of BSTFA (bis(trimethylsilyl)trifluoroacetamide) (or BSA: bistrimethylsilyl)-acetamide) were added at room temperature and under inert gas, and the mixture was heated under reflux for 2 hours. 9 mmol of the desired sulfonyl acid chloride dissolved in 30 ml of acetonitrile were added to this solution (for example 2.84 g of 4-chlorobiphenylethanesulfonyl chloride), and again heated under reflux for 3 hours. After the reaction mixture had cooled, aqueous 1 N HCl was added and, after stirring for 1 hour, the solvent was removed under reduced pressure and, after addition of ethyl acetate or chloroform, the organic phase was separated off, extracted, washed with saturated NaCl solution, dried over Na₂SO₄ and concentrated under reduced pressure. Depending on the purity of the reaction product it could be directly reacted further or was chromatographed on silica gel before further reaction.

### General Methods B1-B2: Reaction of the N-sulfonyltetrahydroisoquinoline-1-carboxylic Acids to Give the Corresponding N-sulfonyltetrahydroisoquinoline-1-hydroxamic Acids

General Method B1

1.0 eq. of an N-sulfonyltetrahydroisoquinoline-1-carboxylic acid were dissolved in abs. DMF (20 ml/mmol), and 1.5 eq. of N-ethylmorpholine were added. At −15° C., 1.2 eq. of ethyl chloroformate are added and stirred at the same temperature for 0.5 hours. Subsequently, 5.0 eq. of O-trimethylsilylhydroxylamine are added, and the cooling bath is removed. After check of the reaction (TLC, LCMS) indicates complete conversion, the mixture is concentrated in vacuo and the residue is taken up in H₂O. A pH of 2-3 is adjusted with citric acid or 2 N HCl, and three extractions are carried out with ethyl acetate. The combined organic phases are dried with MgSO₄ and concentrated. Chromatography on silica gel affords the desired N-sulfonyl-tetrahydroisoquinoline-1-hydroxamic acid.

General Method B2

An N-sulfonyltetrahydroisoquinoline-1-carboxylic acid was introduced into dry chloroform (5 ml/0.5 mmol) and, at room temperature, 3 eq. of oxalyl chloride were added. It was then heated at 45° C. for about 30 minutes. The solvent was then distilled off under reduced pressure, and the residue was taken up in dry toluene and again evaporated several times. The resulting N-sulfonyltetrahydroisoquinoline-1-carbonyl chloride was taken up in chloroform (10 ml/0.5 mmol) and, at room temperature, 3 eq. of O-trimethylsilylhydroxylamine were added. After a reaction time of at least 30 minutes (check of the reaction by HPLC-MS), the reaction mixture was evaporated under reduced pressure. Chromatography of the residue on silica gel affords the desired N-sulfonyltetrahydroisoquinoline-1-hydroxamic acid.

Starting from the described scaffold building blocks A, B, C, D, D1, D2, E or F and the sulfonyl chlorides indicated in each case, the following examples of hydroxamic acid compounds (Table 1) were prepared by general methods (GM) A and B:

TABLE 1

| | | Scaf- | | | |
|---|---|---|---|---|---|
| Comp. | Hydroxamic acid | fold | GM | Sulfonyl chloride | Carboxylic acid |
| 3 | 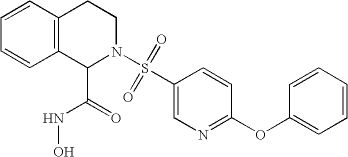 | D | A2;B1 | 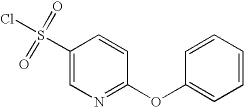 | 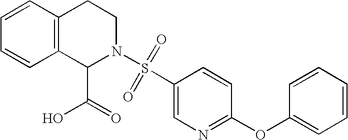 |
| 4 | 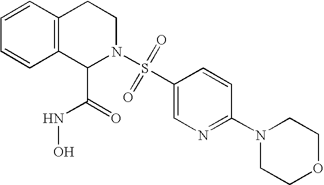 | D | A2;B1 | 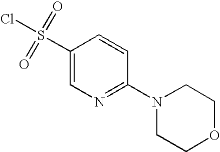 | 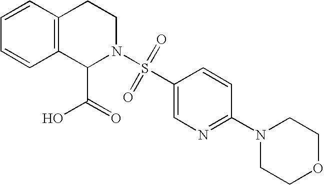 |
| 5 | 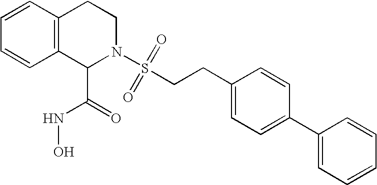 | D | A3;B1 | 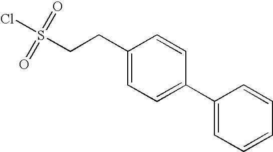 | 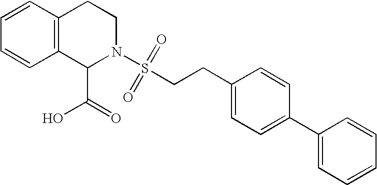 |
| 6 | 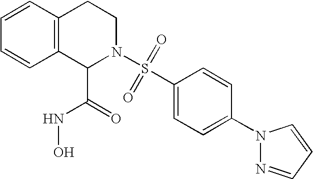 | D | A3;B1 | 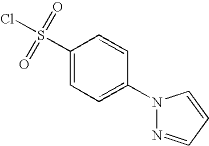 | 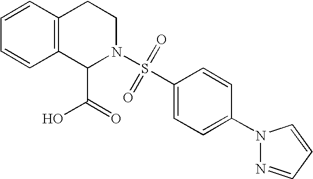 |
| 20 | 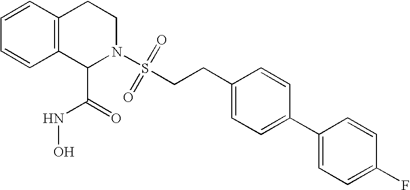 | D | A2;B1 | 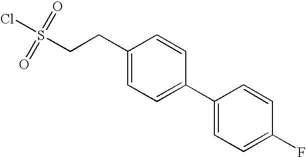 | 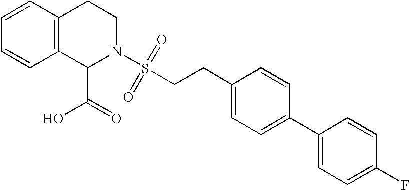 |
| 21 | 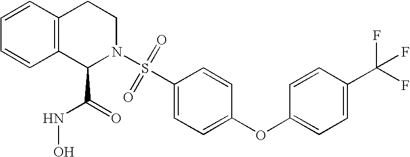 | D1 | A2;B2 | 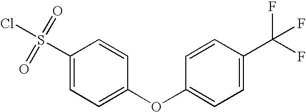 | 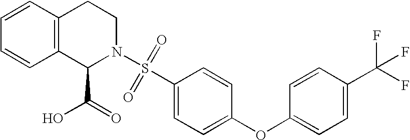 |
| 23 | 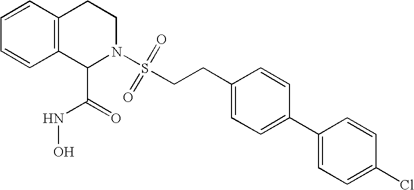 | D | A3;B1 | 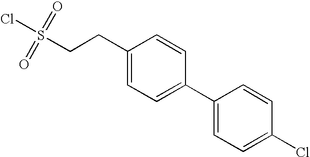 | 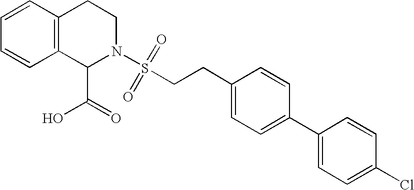 |
| 24 | 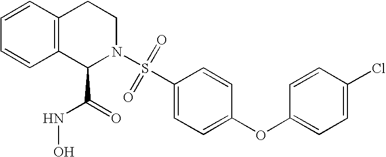 | D1 | A1;B1 | 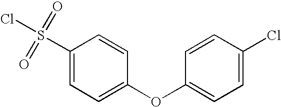 | 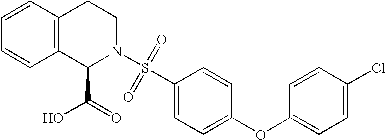 |
| 25 | 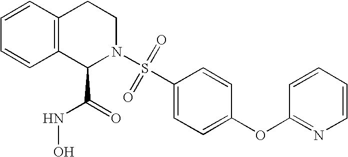 | D1 | A2;B2 | 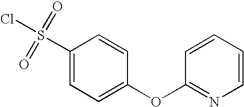 | 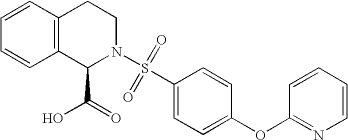 |
| 26 | 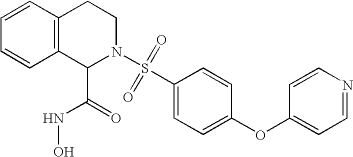 | D | A2;B2 | 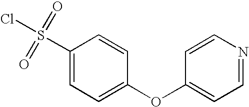 | 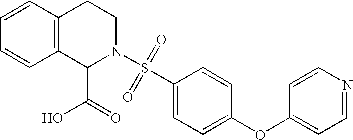 |
| 28 | 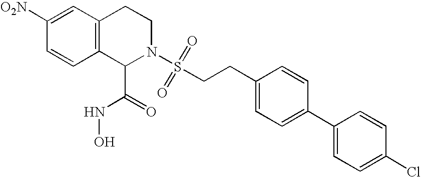 | E | A3;B1 | 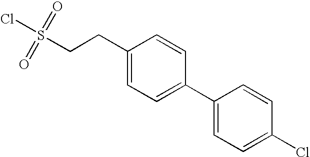 | 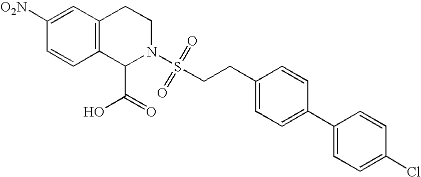 |
| 31 | 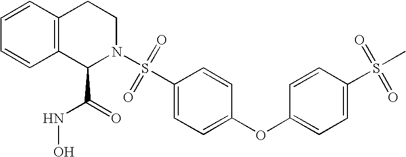 | D1 | A2;B1 | 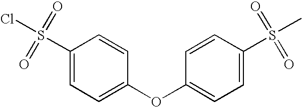 | 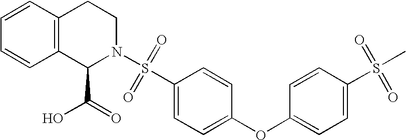 |
| 32 | 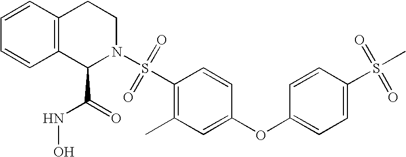 | D1 | A2;B1 | 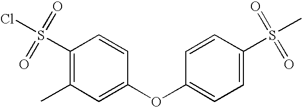 | 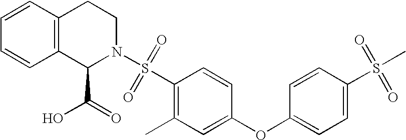 |
| 33 | 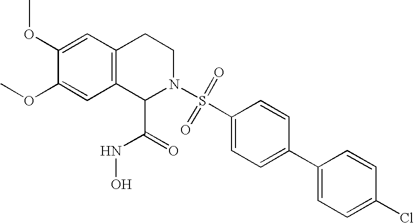 | F | A2;B1 | 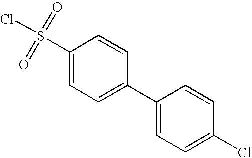 | 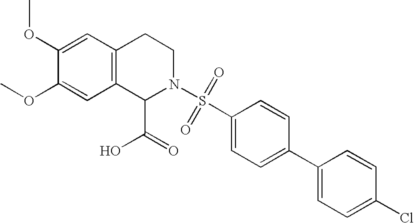 |
| 34 | 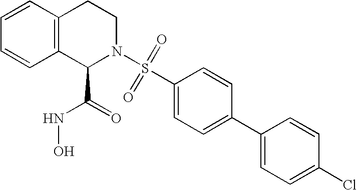 | D1 | A2;B1 | 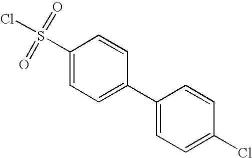 | 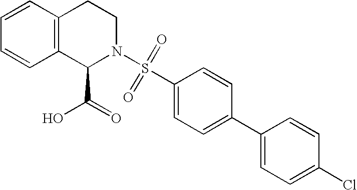 |
| 35 | 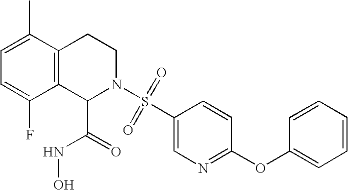 | A | A1;B1 | 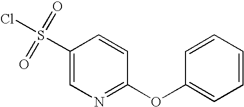 | 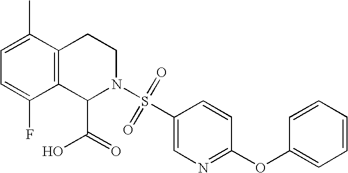 |
| 36 | 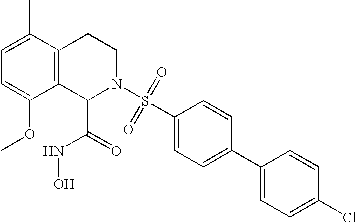 | C | A1;B1 | 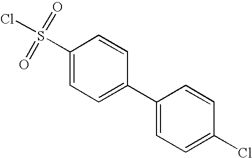 | 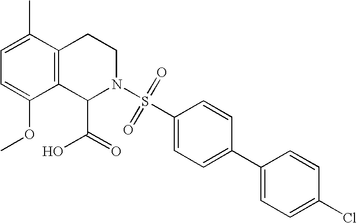 |
| 37 | 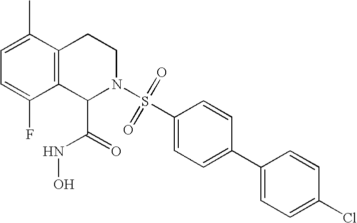 | A | A1;B1 | 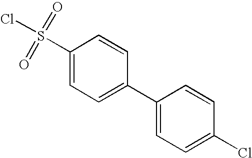 | 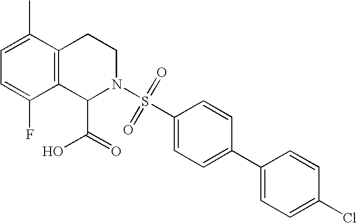 |
| 44 | 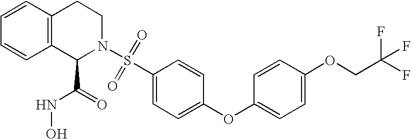 | D1 | A3;B2 | 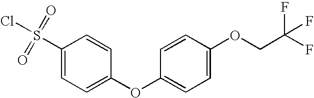 | 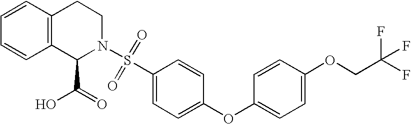 |
| 47 | 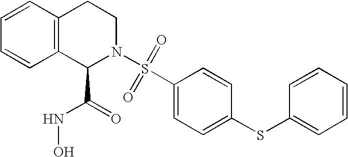 | D1 | A2;B2 | 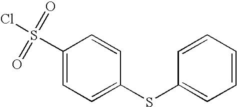 | 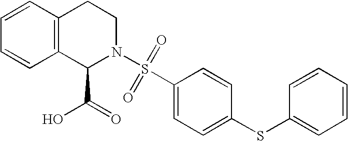 |
| 48 | 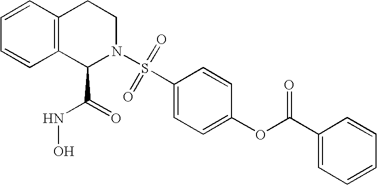 | D1 | A2;B2 | 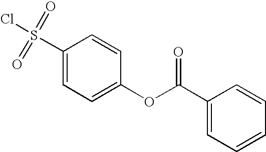 | 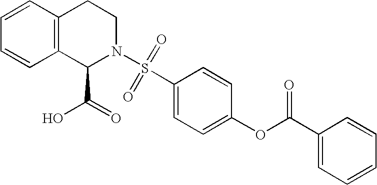 |
| 49 | 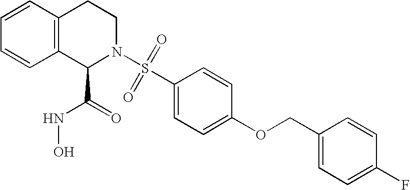 | D1 | A2;B2 | 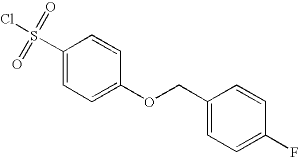 | 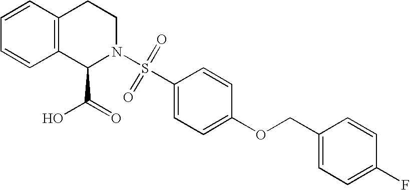 |
| 50 | 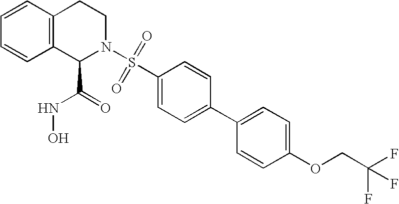 | D1 | A3;B2 | 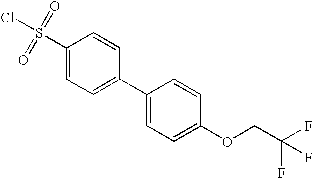 | 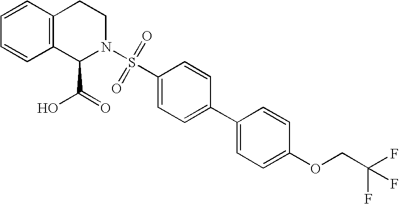 |
| 57 | 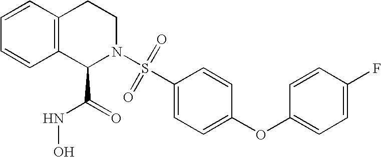 | D1 | A1;B1 | 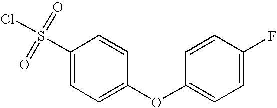 | 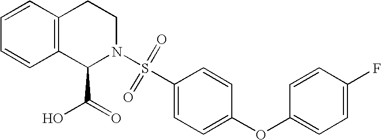 |
| 58 | 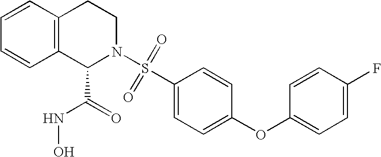 | D2 | A1;B1 | 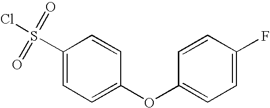 | 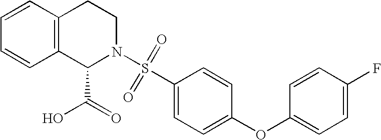 |
| 74 | 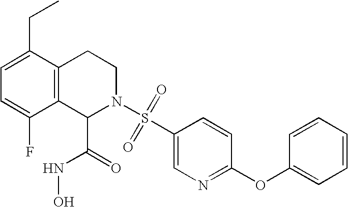 | B | A1;B1 | 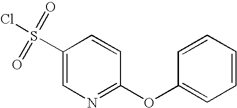 | 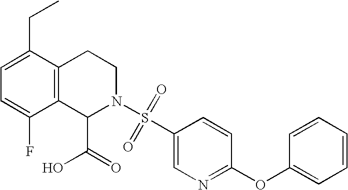 |
| 75 | 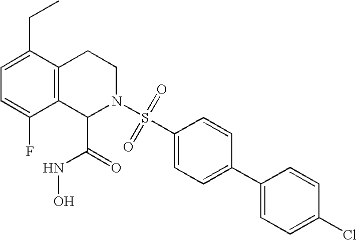 | B | A1;B1 | 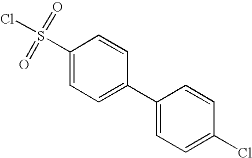 | 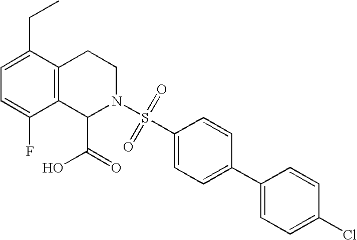 |
| 78 | 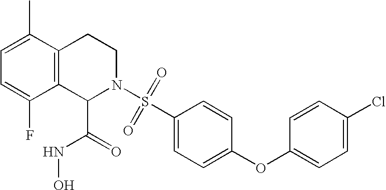 | A | A1;B1 | 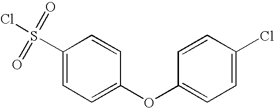 | 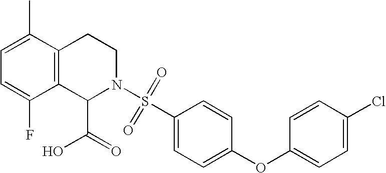 |
| 79 | 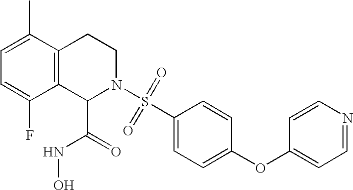 | A | A1;B1 | 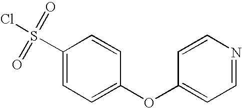 | 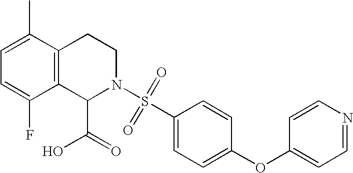 |
| 80 | 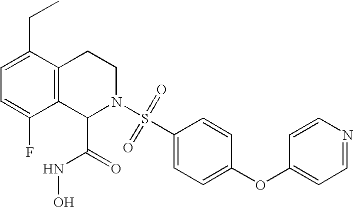 | B | A1;B1 | 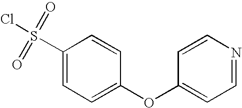 | 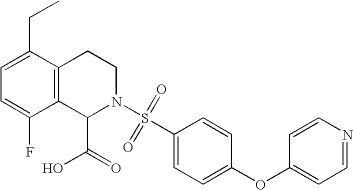 |
| 82 | 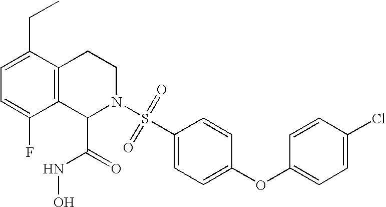 | B | A1;B1 | 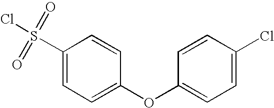 | 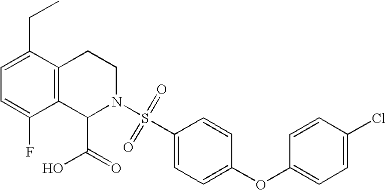 |
| 87 | 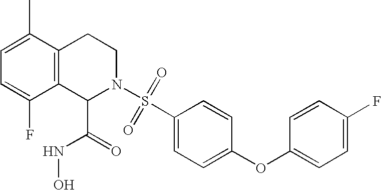 | A | A1;B1 | 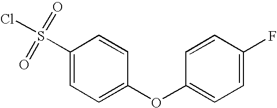 | 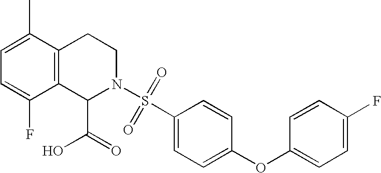 |
| (H) | 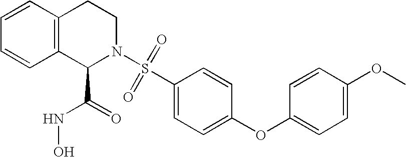 | D1 | A2;B2 | 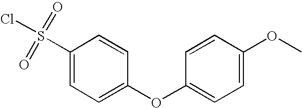 | 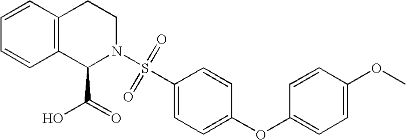 |

Synthesis of Chloropyridines:

### Example 10

### Chloropyridine CP-A (2-(6-chloropyridine-3-sulfonyl)-1,2,3,4-tetrahydro-isoquinoline-1-carboxylic acid)

8.0 g (37.4 mmol) of tetrahydroisoquinoline-1-carboxylic acid hydrochloride were introduced into 160 ml of acetonitrile and, after addition of 3.79 g (37.4 mmol) of triethylamine and 9.9 g (48.7 mmol) of BSA, heated under reflux for two hours. After cooling to 0° C., a further 3.79 g (37.4 mmol) of triethylamine and a solution of 9.5 g (44.9 mmol) of 2-chloropyridin-5-sulfonyl chloride (obtainable according to German patent No. 597452) in 160 ml of acetonitrile was added, and the mixture was again heated under reflux for 1.5 hours. The mixture was worked up by allowing to cool to room temperature and, after addition of 80 ml of 1 N HCl, stirring at room temperature for one hour and concentrating in vacuo. The residue was taken up in H₂O and extracted four times with ethyl acetate. The combined ethyl acetate phases were dried with MgSO₄, and the solvent was removed in vacuo. Chromatography twice on silica gel (dichloromethane/methanol 4:1) affords 7.02 g of the title compound. Yield: 53%.

### Example 11

### Chloropyridine CP-A1 (D-2-(6-chloropyridine-3-sulfonyl)-1,2,3,4-tetrahydroisoquinoline-1-carboxylic acid)

2.5 g (11.7 mmol) of D-tetrahydroisoquinoline-1-carboxylic acid were introduced together with 3.04 (23.5 mmol) of diisopropylethylamine into 40 ml of dichloromethane, and a solution of 3.44 g (16.9 mmol) of BSA in 10 ml of dichloromethane was added dropwise. Heating under reflux for two hours was followed by cooling to 0° C. and addition of a solution of 3.75 g (37.5 mmol) of 2-chloropyridine-5-sulfonyl chloride in 10 ml of dichloromethane. After one hour at room temperature, the mixture washed with H₂O, dried with MgSO₄ and concentrated. Chromatography on silica gel (ethyl acetate/methanol 5:1) afforded 1.0 g of the title compound. Yield: 24%.

### Example 12

### Chloropyridine CP—B (2-(6-chloropyridine-3-sulfonyl)-5-fluoro-8-methyl-1,2,3,4-tetrahydroisoquinoline-1-carboxylic acid)

A solution of 692 mg (3.4 mmol) of BSA in 10 ml of dichloromethane was added dropwise to a solution of 418 mg (1.7 mmol) of 8-methyl-5-fluorotetrahydro-isoquinoline-1-carboxylic acid hydrochloride (Scaffold A) and 660 mg (5.1 mmol) of diisopropylethylamine in 15 ml of dichloromethane at room temperature, and the mixture was stirred at room temperature for two hours. Then, at 0° C., a solution of 443 mg (2.04 mmol) of 2-chloropyridine-5-sulfonyl chloride in 10 ml of dichloromethane was added dropwise, and the mixture was stirred at room temperature for three hours. After standing overnight, it washed three times with 1 N HCl, dried with Na₂SO₄ and concentrated. Chromatography on silica gel (dichloromethane/methanol 95:5) affords 288 mg of the title compound. Yield: 44%.

### General Method C: Reaction of the Chloropyridines CP-A, CP-A1 or B to give the Corresponding N-phenoxypyridinesulfonyltetrahydroisoquinoline-1-carboxylic Acids

2.5 eq. of a phenol building block were dissolved in abs. DMF (2 mmol/10 ml) and, at room temperature, 4.0 eq. of NaH were added. After stirring at room temperature with exclusion of moisture for 30 min, 1.0 eq. of the appropriate chloropyridine CP-A, CP-A1 or CP—B was added and heated at 100° C. for two hours. For workup, the solvent was removed in vacuo, the residue was taken up in H₂O, and a pH of about 4 was adjusted with 2 N HCl. Five extractions with ethyl acetate were carried out, and the ethyl acetate phases were dried with MgSO₄ and concentrated. Chromatography on silica gel affords the desired phenoxypyridinesulfonyltetrahydroisoquinoline-1-carboxylic acids.

The analogous hydroxamic acids were synthesized in analogy to general method B1 (reaction of the N-sulfonyltetrahydroisoquinoline-1-carboxylic acids to give the corresponding N-sulfonyltetrahydroisoquinoline-1-hydroxamic acids). The following examples of hydroxamic acid compounds (Table 2) were prepared by general methods C and B1 starting from chloropyridine (CP)CP-A, CP-A1 or CP—B, where “1” means that KO^{t}Bu was used instead of NaH as base, and dimethoxyethane was used as solvent, and stirring took place at 80° C. overnight, in general method C:

TABLE 2

| Comp. | Hydroxamic acid | CP | Phenol building block | Carboxylic acid |
|---|---|---|---|---|
| 27 | 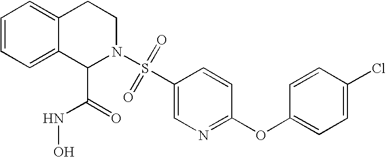 | A | 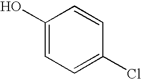 | 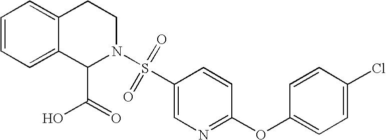 |
| 35¹ | 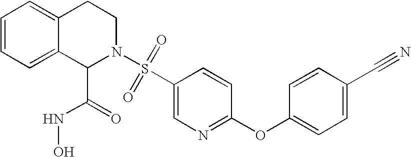 | A | 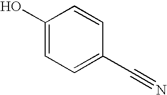 | 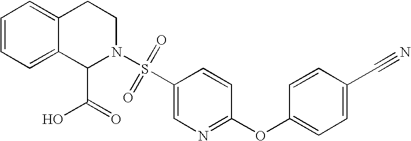 |
| 36¹ | 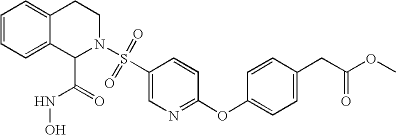 | A | 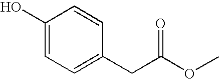 | 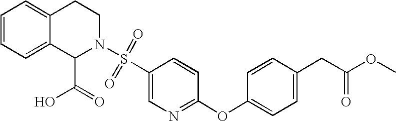 |
| 37¹ | 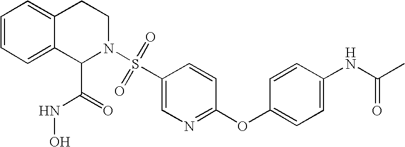 | A | 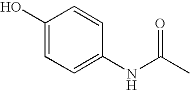 | 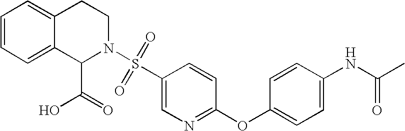 |
| 38¹ | 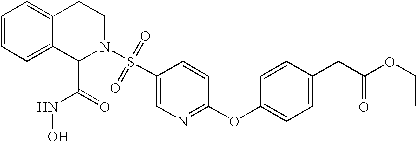 | A | 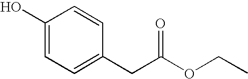 | 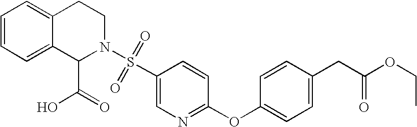 |
| 39¹ | 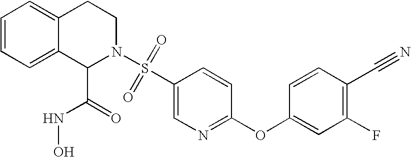 | A | 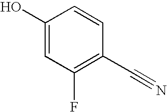 | 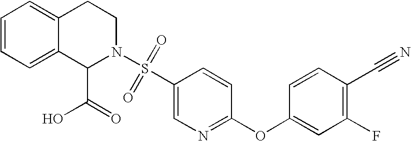 |
| 40¹ | 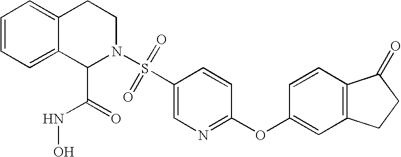 | A | 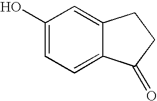 | 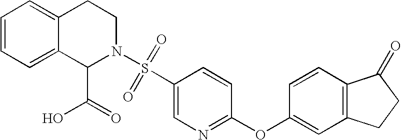 |
| 41¹ | 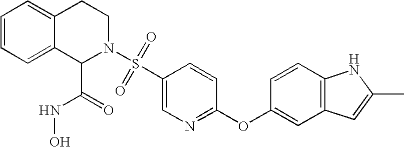 | A | 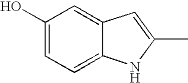 | 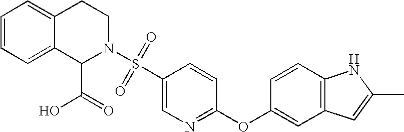 |
| 42 | 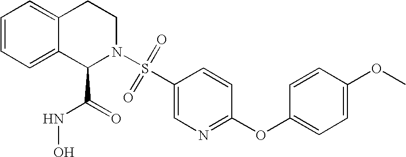 | A1 | 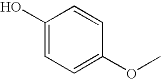 | 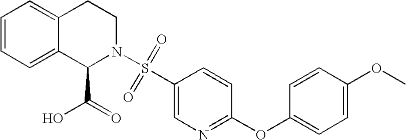 |
| 43 | 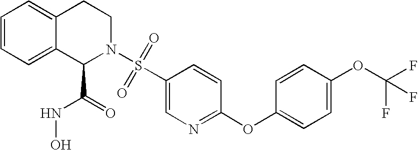 | A1 | 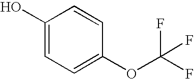 | 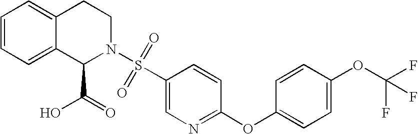 |
| 45 | 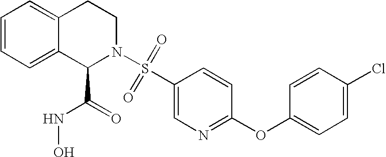 | A1 | 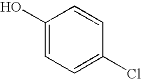 | 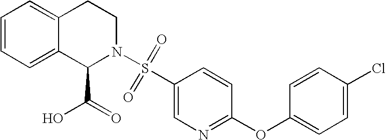 |
| 46 | 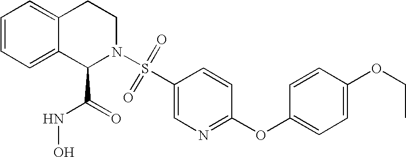 | A1 | 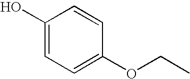 | 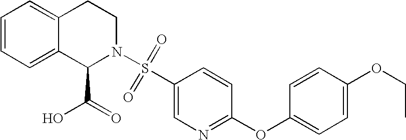 |
| 76 | 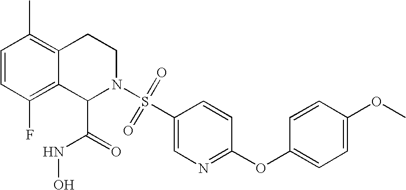 | B | 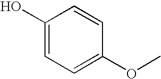 | 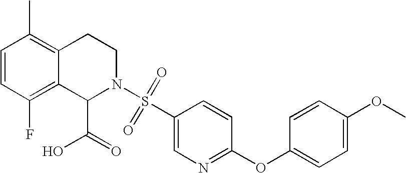 |
| 77 | 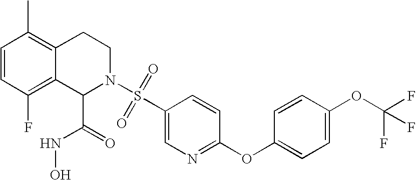 | B | 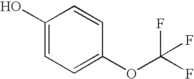 | 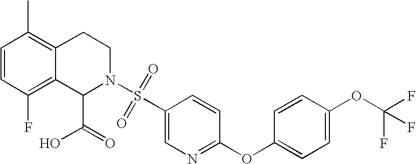 |
| 81 | 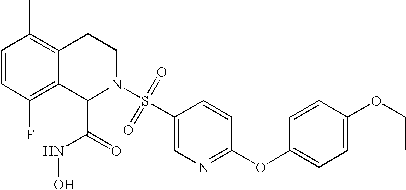 | B | 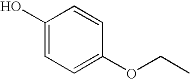 | 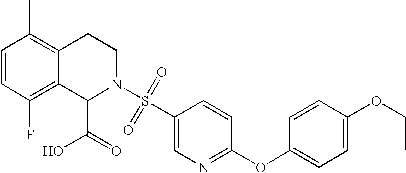 |

### Example 13

### Separation of Diastereomers of the Compound of Example 78 (2-[4-(4-chlorophenoxy)benzenesulfonyl]-5-methyl-8-fluoro-1,2,3,4-tetrahydroisoquinoline-1-hydroxycarboxamide): enantiomer 78A and enantiomer 78B

120 mg of 2-[4-(4-chlorophenoxy)benzenesulfonyl]-5-methyl-8-fluoro-1,2,3,4-tetrahydroisoquinoline-1-hydroxycarboxamide (compound of Example 78) were separated on a chiral phase. Detection of the two enantiomers was carried out on an analytical chiral phase (without assigning the absolute stereochemistry).

Chiral column: Chiralpak AD-H/44 250×4.6 mm;

Mobile phase: ethanol:methanol 1:1;

Flow rate: 1 ml/min;

Running time: 24 min;

Temperature: 30° C.

Rₜ (enantiomer 78A): 5.92 min; yield: 49 mg.

Rₜ (enantiomer 78B): 20.35 min; yield: 47 mg.

### Example 14

### Characterization of the Compounds of Examples 1-50

The compounds of the examples were characterized by determining their retention times and molecular peaks (Table 3).

TABLE 3
Analytical data on the compounds
of the examples in Tables 1 and 2

| | Retention time*⁾ | Mass (M + H)⁺ | LCMS |
|---|---|---|---|
| Comp. | Rₜ [min] | [g/mol] | method |
| 3 | 3.34 | 425.10 | A |
| 4 | 2.58 | 418.13 | A |
| 5 | 3.85 | 436.15 | A |
| 6 | 2.89 | 398.1 | A |
| 20 | 1.75 | 468.55 | G |
| 21 | 1.62 | 493.10 | D |
| 23 | 1.67 | 471.15 | D |
| 24 | 2.24 | 461.10 | B |
| 25 | 3.34 | 425.11 | A |
| 26 | 1.54 | 425.1 | F |
| 27 | 2.41 | 460.00 | A |
| 28 | 1.67 | 517.1 | D |
| 31 | 1.19**⁾ | 503.5 | I |
| 32 | 1.34 | 517.1 | D |
| 33 | 1.49 | 503.0 | D |
| 34 | 1.57 | 444.1 | D |
| 35 | 1.94 | 458.08 | B |
| 35 | 1.77 | 451.19 | C |
| 36 | 2.26 | 490.97 | B |
| 36 | 1.87 | 498.12 | B |
| 37 | 1.61 | 475.05 | D |
| 37 | 1.61 | 483.21 | C |
| 38 | 1.99 | 512.14 | B |
| 39 | 1.81 | 469.17 | C |
| 40 | 1.72 | 480.20 | C |
| 41 | 1.88 | 479.13 | B |
| 42 | 1.93 | 456.18 | B |
| 43 | 2.27 | 510.04 | B |
| 44 | 2.44 | 522.11 | B |
| 45 | 1.97 | 459.98 | B |
| 46 | 1.96 | 470.02 | B |
| 47 | 1.86 | 440.08 | H |
| 48 | 1.19 | 334.1 (-benzoate) | D |
| 49 | 2.08 | 456.12 | B |
| 50 | 2.13 | 506.11 | B |
| 57 | 2.06 | 443.15 | B |
| 58 | 2.09 | 443.15 | B |
| 74 | 2.07 | 472.08 | B |
| 75 | 2.04 | 489.19 | C |
| 76 | 1.82 | 487.87 | E |
| 77 | 1.90 | 541.82 | E |
| 78 | 2.46 | 490.98 | B |
| 78A | 1.89 | 491.36 | E |
| 78B | 1.90 | 491.36 | E |
| 79 | 1.16 | 457.98 | B |
| 80 | 0.97 | 472.15 | D |
| 81 | 2.05 | 502.02 | B |
| 82 | 2.44 | 505.08 | B |
| 87 | 1.49 | 475.10 | I |
| (H) | 2.38 | 454.12 | A |

LCMS methods:
A

| | |
|---|---|
| Stationary phase: | Col Merck Purosphere 55 × 2 mm; |
| Mobile Phase: | (ACN + 0.08% FA):(H₂O + 0.1% FA), |
| | 5:95 (0 min) to 95:5 (5 min) to 95:5 (7 min); |
| Flow rate: | 1 ml/min; |
| Temperature: | 30° C. |

B

| | |
|---|---|
| Stationary phase: | Col YMC J'sphere 33 × 2 mm; |
| Mobile Phase: | (ACN + 0.05% TFA):(H₂O + 0.05% TFA), |
| | 5:95 (0 min) to 95:5 (3.4 min) to 95:5 (4.4 min); |
| Flow rate: | 1 ml/min; |
| Temperature: | 30° C. |

C

| | |
|---|---|
| Stationary phase: | Col Uptisphere ODB 33 × 2 mm; |
| Mobile Phase: | (ACN + 0.08% FA):(H₂O + 0.1% FA), |
| | 5:95 (0 min) to 95:5 (2.5 min) to 95:5 (3 min); |
| Flow rate: | 1 ml/min; |
| Temperature: | 30° C. |

D

| | |
|---|---|
| Stationary phase: | Col YMC J'sphere ODS H80 20 × 2 mm; |
| Mobile Phase: | ACN:(H₂O + 0.05% TFA), |
| | 4:96 (0 min) to 95:5 (2.0 min) to 95:5 (2.4 min); |
| Flow rate: | 1 ml/min; |
| Temperature: | 30° C. |

E

| | |
|---|---|
| Stationary phase: | Col YMC J'sphere 33 × 2 mm; |
| Mobile Phase: | (ACN + 0.05% TFA):(H₂O + 0.05% TFA), |
| | 5:95 (0 min) to 95:5 (2.5 min) to 95:5 (3 min); |
| Flow rate: | 1 ml/min; |
| Temperature: | 30° C. |

F

| | |
|---|---|
| Stationary phase: | Col Merck Purosphere 55 × 2 mm; |
| Mobile Phase: | (ACN + 0.05% TFA):(H₂O + 0.05% TFA), |
| | 5:95 (0 min) to 95:5 (3.4 min) to 95:5 (4.4 min); |
| Flow rate: | 0.75 ml/min; |
| Temperature: | 30° C. |

G

| | |
|---|---|
| Stationary phase: | Col YMC; |
| Mobile Phase: | (ACN + 0.08% FA):(H₂O + 0.1% FA), |
| | 5:95 (0 min) to 95:5 (2.5 min) to 95:5 (4 min); |
| Flow rate: | not determined; |
| Temperature: | 30° C. |

H

| | |
|---|---|
| Stationary phase: | Col YMY J'sphere 33 × 2 mm; |
| Mobile Phase: | (ACN + 0.05% TFA):(H₂O + 0.05% TFA), |
| | 5:95 (0 min) to 95:5 (2.5 min) to 95:5 (3.0 min); |
| Flow rate: | 1 ml/min; |
| Temperature: | 30° C. |

I

| | |
|---|---|
| Stationary phase: | Col. YMC J'sphere ODS H80 20 × 2 mm; |
| Mobile Phase: | (ACN + 0.05% TFA):(H₂O + 0.05% TFA), |
| | 4:96 (0 min) to 95:5 (2.0 min) to 95:5 (2.4 min); |
| Flow rate: | 1 ml/min; |
| Temperature: | 30° C. |

*⁾Retention time based on the mass spectrum;
**⁾Retention time based on the UV spectrum.

### Example 15

### Determination of the Enzymatic Activity of the Catalytic Domain of Human Collagenase-1 (MMP-1)

MMP-1 was obtained as inactive Pro enzyme from Biocol, Potsdam (catalog No. MMP1). Activation of the Pro enzyme: 2 parts by volume of Pro enzyme are incubated with 1 part by volume of APMA solution at 37° C. for 1 hour. The APMA solution is prepared from a 10 mmol/l p-aminophenylmercuric acetate solution in 0.1 mmol/l NaOH by dilution with 3 parts by volume of tris/HCl buffer pH7.5 (see below). The pH is adjusted to between 7.0 and 7.5 by adding 1 mmol/l HCl. After activation of the enzyme, it is diluted with the tris/HCl buffer to a concentration of 2.5 μg/ml.

The enzymic activity is measured by incubating 10 μl of enzyme solution with 10 μl of a 3% strength (v/v) buffered dimethyl sulfoxide solution (reaction 1) for 15 minutes. The enzyme inhibitor activity is measured by incubating 10 μl of enzyme solution with 10 μl of a 3% strength (v/v) buffered dimethyl sulfoxide solution which contains the enzyme inhibitor (reaction 2).

The enzymic reaction both in the case of reaction 1 and in the case of reaction 2 is followed after addition of 10 μl of a 3% strength (v/v) aqueous dimethyl sulfoxide solution which contains 0.3 mmol/l of the substrate by fluorescence spectroscopy (328 nm (extinction)/393 nm (emission)), and the enzymic activity is presented as increase in extinction per minute.

The effect of the inhibitor is calculated as percentage inhibition by the following formula:
% inhibition=100−[(increase in extinction/minute in reaction 2)/increase in extinction/minute in reaction 1)×100].

The IC₅₀, i.e. the inhibitor concentration necessary for 50% inhibition of the enzymic activity, is determined graphically by plotting the percentage inhibitions at various inhibitor concentrations.

The buffer solution contains 0.05% Brij (Sigma, Deisenhofen, Germany) and 0.1 mol/l tris/HCl, 0.1 mol/l NaCl, 0.01 mol/l CaCl₂ (pH=7.5).

The enzyme solution contains 2.5 μg/ml of the enzyme domain.

The substrate solution contains 0.3 mmol/l of the fluorogenic substrate (7-methoxycoumarin-4-yl)acetyl-Pro-Leu-Gly-Leu-3-(2′,4′-dinitrophenyl)-L-2,3-diaminopropionyl-Ala-Arg-NH₂ (Bachem, Heidelberg, Germany).

### Example 16

### Preparation and Determination of the Enzymatic Activity of the Catalytic Domain of Human Stromelysin (MMP-3) and of Neutrophil Collagenase (MMP-8)

The two enzymes stromelysin (MMP-3) and neutrophil collagenase (MMP-8) were prepared by the method of Ye et al. (Biochemistry; 31 (1992) pages 11 231-11 235). The enzymic activity or the effect of the enzyme inhibitor was measured by incubating 10 μl of enzyme solution with 10 μl of a 3% strength (v/v) buffered dimethyl sulfoxide solution which contained the enzyme inhibitor where appropriate, for 15 minutes. After addition of 10 μl of a 3% strength (v/v) aqueous dimethyl sulfoxide solution which contained 1 mmol/l of the substrate, the enzymic reaction was followed by fluorescence spectroscopy (328 nm (ex)/393 nm(em)). The enzymic activity is presented as increase in extinction/minute.

The IC₅₀ values listed in Table 4 were determined as the inhibitor concentrations leading in each case to 50% inhibition of the enzyme. The buffer solution contained 0.05% Brij (Sigma, Deisenhofen, Germany) and 0.1 mol/l Tris/HCl, 0.1 mol/l NaCl, 0.01 mol/l CaCl₂ and 0.1 mol/l piperazine-N,N′-bis[2-ethanesulfonic acid] (pH=7.5).

The MMP-3 enzyme solution contained 2.3 μg/ml and the MMP-8 enzyme solution 0.6 μg/ml of one of the enzyme domains prepared by the method of Ye et al. The substrate solution contained 1 mmol/l of the fluorogenic substrate (7-methoxycoumarin-4-yl)acetyl-Pro-Leu-Gly-Leu-3-(2′,4′-dinitrophenyl)-L-2,3-diaminopropionyl-Ala-Arg-NH₂ (Bachem, Heidelberg, Germany).

### Example 17

### Determination of the Enzymatic Activity of the Catalytic Domain of Human Collagenase-3 (MMP-13)

MMP-13 was obtained as inactive proenzyme from INVITEK, Berlin. Activation of the proenzyme: 2 parts by volume of proenzyme were incubated with 1 part by volume of APMA solution at 37° C. for 1.5 hours. The APMA solution was prepared from a 10 mmol/l p-aminophenylmercuric acetate solution in 0.1 mmol/l NaOH by dilution with 3 parts by volume of Tris/HCl buffer pH 7.5 (see below). The pH was adjusted to between 7.0 and 7.5 by adding 1 mmol/l HCl. After activation of the enzyme it was diluted with the Tris/HCl buffer to a concentration of 1.67 μg/ml.

The enzymic activity was measured by incubating 10 μl of enzyme solution with 10 μl of a 3% strength (v/v) buffered dimethyl sulfoxide solution (reaction 1) for 15 minutes. The enzyme inhibitor activity was measured by incubating 10 μl of enzyme solution with 10 μl of a 3% strength (v/v) buffered dimethyl sulfoxide solution which contained the enzyme inhibitor (reaction 2).

The enzymic reaction both in the case of reaction 1 and in the case of reaction 2 was followed after addition of 10 μl of a 3% strength (v/v) aqueous dimethyl sulfoxide solution which contained 0.075 mmol/l of the substrate by fluorescence spectroscopy (328 nm (extinction)/393 nm (emission)).

The enzymic activity has been presented as increase in extinction/minute. The effect of the inhibitor was calculated as percentage inhibition by the following formula:
% inhibition=100−[(increase in extinction/minute in reaction 2)/(increase in extinction/minute in reaction 1)×100].

The IC₅₀, which is the concentration of inhibitor which is necessary for 50% inhibition of the enzymic activity, was determined graphically by plotting the percentage inhibitions at various inhibitor concentrations.

The buffer solution contained 0.05% Brij (Sigma, Deisenhofen, Germany) and 0.1 mol/l Tris/HCl, 0.1 mol/l NaCl, 0.01 mol/l CaCl₂ (pH=7.5). The enzyme solution contained 1.67 μg/ml of the enzyme domain. The substrate solution contained 0.075 mmol/l of the fluorogenic substrate (7-methoxycoumarin-4-yl)acetyl-Pro-Leu-Gly-Leu-3-(2′,4′-dinitrophenyl)-L-2,3-diaminopropionyl-Ala-Arg-NH₂ (Bachem, Heidelberg, Germany).

### Example 18

### Determination of the Enzymatic Activity of the Catalytic Domain of Human Gelatinase-A (MMP-2)

MMP-2 was obtained as inactive proenzyme from INVITEK, Berlin. Activation of the proenzyme: 2 parts by volume of proenzyme were incubated with 1 part by volume of APMA solution at 37° C. for 0.5 hours. The APMA solution was prepared from a 10 mmol/l p-aminophenylmercuric acetate solution in 0.1 mmol/l NaOH by dilution with 3 parts by volume of Tris/HCl buffer pH 7.5 (see below). The pH was adjusted to between 7.0 and 7.5 by adding 1 mmol/l HCl. After activation of the enzyme it was diluted with the Tris/HCl buffer to a concentration of 0.83 μg/ml.

The enzymic activity was measured by incubating 10 μl of enzyme solution with 10 μl of a 3% strength (v/v) buffered dimethyl sulfoxide solution (reaction 1) for 15 minutes. The enzyme inhibitor activity was measured by incubating 10 μl of enzyme solution with 10 μl of a 3% strength (v/v) buffered dimethyl sulfoxide solution which contained the enzyme inhibitor (reaction 2).

The enzymic reaction both in the case of reaction 1 and in the case of reaction 2 was followed after addition of 10 μl of a 3% strength (v/v) aqueous dimethyl sulfoxide solution which contained 0.3 mmol/l of the substrate by fluorescence spectroscopy (328 nm (extinction)/393 nm (emission)).

The enzymic activity has been presented as increase in extinction/minute.

The effect of the inhibitor was calculated as percentage inhibition by the following formula:
% inhibition=100−[(increase in extinction/minute in reaction 2)/(increase in extinction/minute in reaction 1)×100].

The IC₅₀, which is the concentration of inhibitor which is necessary for 50% inhibition of the enzymic activity, was determined graphically by plotting the percentage inhibitions at various inhibitor concentrations.

The buffer solution contained 0.05% Brij (Sigma, Deisenhofen, Germany) and 0.1 mol/l Tris/HCl, 0.1 mol/l NaCl, 0.01 mol/l CaCl₂ (pH=7.5). The enzyme solution contained 10.83 μg/ml of the enzyme domain. The substrate solution contained 0.3 mmol/l of the fluorogenic substrate (7-methoxycoumarin-4-yl)acetyl-Pro-Leu-Gly-Leu-3-(2′,4′-dinitrophenyl)-L-2,3-diaminopropionyl-Ala-Arg-NH₂ (Bachem, Heidelberg, Germany).

### Example 19

### Determination of the Enzymatic Activity of the Catalytic Domain of Human Gelatinase-A (MMP-9)

MMP-9 was obtained as inactive proenzyme from Roche, Mannheim. Activation of the proenzyme:

2 parts by volume of proenzyme were incubated with 1 part by volume of APMA solution at 37° C. for 4 hours. The APMA solution was prepared from a 10 mmol/l p-aminophenylmercuric acetate solution in 0.1 mmol/l NaOH by dilution with 3 parts by volume of Tris/HCl buffer pH 7.5 (see below). The pH was adjusted to between 7.0 and 7.5 by adding 1 mmol/l HCl. After activation of the enzyme it was diluted with the Tris/HCl buffer to a concentration of 4.2 mU/ml.

The enzymic activity was measured by incubating 10 μl of enzyme solution with 10 μl of a 3% strength (v/v) buffered dimethyl sulfoxide solution (reaction 1) for 15 minutes. The enzyme inhibitor activity was measured by incubating 10 μl of enzyme solution with 10 μl of a 3% strength (v/v) buffered dimethyl sulfoxide solution which contained the enzyme inhibitor (reaction 2).

The enzymic reaction both in the case of reaction 1 and in the case of reaction 2 was followed after addition of 10 μl of a 3% strength (v/v) aqueous dimethyl sulfoxide solution which contained 0.15 mmol/l of the substrate by fluorescence spectroscopy (328 nm (extinction)/393 nm (emission)).

The enzymic activity has been presented as increase in extinction/minute.

The effect of the inhibitor was calculated as percentage inhibition by the following formula:
% inhibition=100−[(increase in extinction/minute in reaction 2)/(increase in extinction/minute in reaction 1)×100].

The IC₅₀, which is the concentration of inhibitor which is necessary for 50% inhibition of the enzymic activity, was determined graphically by plotting the percentage inhibitions at various inhibitor concentrations.

The buffer solution contained 0.05% Brij (Sigma, Deisenhofen, Germany) and 0.1 mol/l Tris/HCl, 0.1 mol/l NaCl, 0.01 mol/l CaCl₂ (pH=7.5). The enzyme solution contained 4.2 mU/ml of the enzyme domain. The substrate solution contained 0.15 mmol/l of the fluorogenic substrate (7-methoxycoumarin-4-yl)acetyl-Pro-Leu-Gly-Leu-3-(2′,4′-dinitrophenyl)-L-2,3-diaminopropionyl-Ala-Arg-NH₂ (Bachem, Heidelberg, Germany).

Table 4 shows the inhibitory profile of selected compounds of the examples as IC₅₀ in nM and the selectivity of inhibition of MMP-9 in relation to MMP-1:

TABLE 4

| Comp. | Structure | MMP-1 | MMP-2 | MMP-3 | MMP-8 | MMP-9 | MMP-13 | MMP9:MMP1 |
|---|---|---|---|---|---|---|---|---|
| 7 | 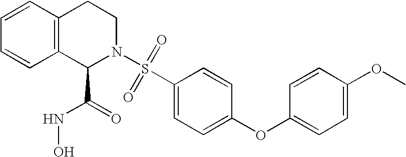 | 60 | 2 | 23 | 2.2 | 1 | 2.2 | 60 |
| 8 | 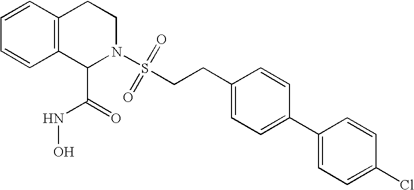 | 5300 | 22 | 220 | 25 | 10 | 22 | 530 |
| 9 | 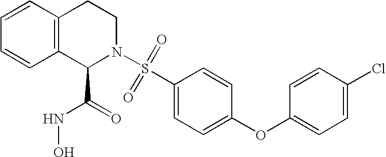 | 42 | 2 | 23 | 2.2 | 1 | 2.1 | 2 |
| 11 | 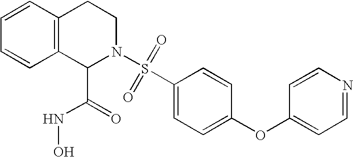 | 500 | 2.3 | 25 | 3 | 1.8 | 2.3 | 78 |
| 12 | 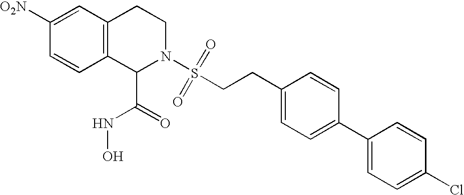 | 700 | 2.2 | 25 | 3.5 | 2 | 2.5 | 350 |
| 15 | 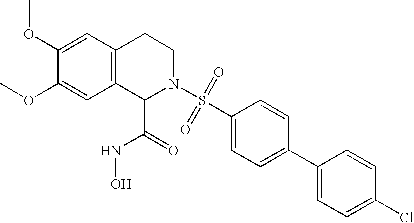 | 300 | 1 | 20 | 2 | 1 | 2 | 300 |
| 18 | 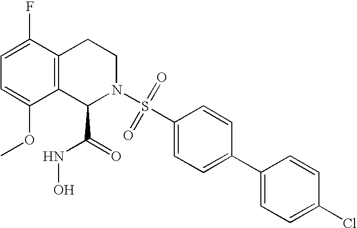 | 4000 | 4.5 | 800 | 14 | 60 | 7.5 | 67 |
| 20 | 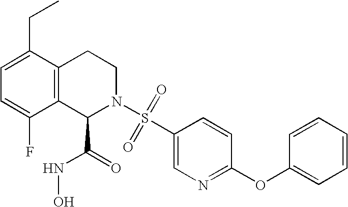 | 300 | 2 | 23 | 2.2 | 1.3 | 2.2 | 231 |
| 21 | 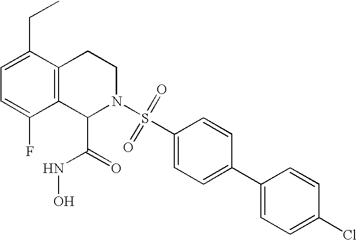 | 1000 | 2.2 | 33 | 3.2 | 3.2 | 2.5 | 313 |
| 22 | 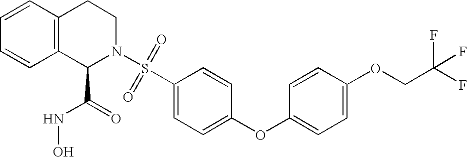 | 1600 | 25 | 520 | 110 | 18 | 130 | 89 |
| 43 | 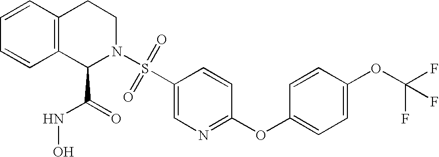 | >10 000 | 23 | 300 | 30 | 20 | 30 | >500 |
| 45 | 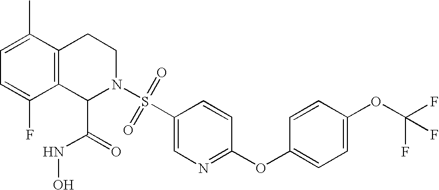 | 2000 | 2 | 45 | 9 | 3 | 8 | 667 |
| 48 | 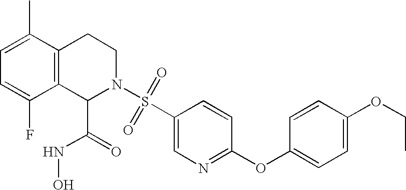 | 89 | 2 | 26 | 4 | 2 | 3 | |

We claim:

1. A compound of formula (I) 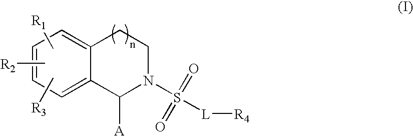
wherein:
R₁, R₂ and R₃ are independently of one another H, F, Cl, Br, I, NO₂, CN, OH, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, —O(C₁-C₆)alkyl, O(C₂-C₆)alkenyl, —O(C₃-C₈)cycloalkyl, —O(C₁-C₄)alkyl-(C₃-C₈)cycloalkyl, —O(C₃-C₈)cycloalkyl-(C₁-C₄)alkyl, —OC(O)—(C₁-C₆)alkyl, —OC(O)—(C₂-C₆)alkenyl, —OC(O)—(C₃-C₈)cycloalkyl, —OC(O)—(C₁-C₄)alkyl-(C₃-C₈)cycloalkyl, —OC(O)—(C₃-C₈)cycloalkyl-(C₁-C₄)alkyl, —C(O)O—(C₁-C₆)alkyl, —C(O)O—(C₂-C₆)alkenyl, C(O)O—(C₃-C₈)cycloalkyl, —C(O)O—(C₁-C₄)alkyl-(C₃-C₈)cycloalkyl, —C(O)O—(C₃-C₈)cycloalkyl-(C₁-C₄)alkyl, —C(O)NR₆R₇, —NR₆R₇ or —NR₆C(O)R₇;
A is —C(O)R₅ or —CH₂SH;
R₅ is OR₆, NR₆R₇ or NR₆OH;
R₆ and R₇ are independently of one another H or (C₁-C₆)alkyl;
n is 0, 1 or 2;
L is —O—, —NR₁₄—, a covalent bond or —(CH₂)_{q}—;
R₁₄ is H or (C₁-C₆)alkyl;
q is 1, 2, 3 or 4;
R₄ is phenyl or (C₅-C₁₄)heteroaryl, each of which is optionally substituted by 1, 2 or 3 substitutents independently of one another selected from the group of F, Cl, Br, I, CN, OH, NO₂, (C₁-C₆)alkyl, (C₂-C₆)alkenyl, (C₂-C₆)alkynyl, (C₃-C₈)cycloalkyl, (C₁-C₄)alkyl-(C₃-C₈)cycloalkyl, —(C₃-C₈)cycloalkyl-(C₁-C₄)alkyl, —O(C₁-C₆)alkyl, —O(C₂-C₆)alkenyl, —O(C₂-C₆)alkynyl, —O(C₃-C₈)cycloalkyl, —O(C₁-C₄)alkyl-(C₃-C₈)cycloalkyl, —O(C₃-C₈)cycloalkyl-(C₁-C₄)alkyl, —O(C₁-C₄)alkyl-O—(C₁-C₆)alkyl or a radical NR₈R₉, and each of which is optionally substituted by a group T-Z;
R₈ and R₉ are defined independently of one another by H, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, (C₂-C₆)alkynyl, —C(O)—V—(C₁-C₆)alkyl, —C(O)—V—(C₂-C₆)alkenyl, —C(O)—V—(C₃-C₈)cycloalkyl, —C(O)—V—(C₁-C₄)alkyl-(C₃-C₈)cycloalkyl, —C(O)—V—(C₃-C₈)cycloalkyl-(C₁-C₄)alkyl or —C(O)—V—(C₂-C₆)alkynyl, or
R₈ and R₉ together with the nitrogen atome to which they are attached form a 5- or 6-membered saturated heterocyclic ring containing one nitrogen;
V is a covalent bond, —O— or —NH—;
T is a covalent bond, —O—, —S—, —O(C₁-C₄)alkyl-, —N(R₁₀)—, —C(O)—, —C(O)O—, —OC(O)—, —C(O)N(R₁₀)—, —N(R₁₀)—C(O)— or —N(R₁₀)—C(O)—N(R₁₁)—;
R₁₀ and R₁₁ are independently of one another H or (C₁-C₄)alkyl;
Z is phenyl, (C₅-C₁₄)heteroaryl, (C₃-C₈)heterocycloalkyl or benzocyclo(C₅-C₇)alken-1-one, each of which is unsubstituted or substituted by 1, 2 or 3 substitutents independently of one another selected from the group of F, Cl, Br, I, CN, OH, NO₂,
(C₁-C₆)alkyl, —SO₂(C₁-C₆)alkyl, —O(C₁-C₄)alkyl-O—(C₁-C₆)alkyl, —(C₁-C₄)alkyl-C(O)—O(C₁-C₆)alkyl, —O(C₁-C₆)alkyl, (C₂-C₆)alkenyl, (C₃-C₈)cycloalkyl, —(C₁-C₄)alkyl-(C₃-C₈)cycloalkyl or —(C₃-C₈)cycloalkyl-(C₁-C₄)alkyl, wherein one or more CH₂ groups in the alkenyl, alkyl or cycloalkyl radicals may be replaced by O or C(O), or
—O(C₂-C₆)alkenyl, —O(C₃-C₈)cycloalkyl, O(C₁-C₄)alkyl-(C₃-C₈)cycloalkyl, O(C₃-C₈)cycloalkyl-(C₁-C₄)alkyl, (C₂-C₆)alkynyl, O(C₂-C₆)alkynyl, or NR₁₂R₁₃,
wherein one or more H atoms in (C₁-C₆)alkyl, (C₁-C₄)alkyl, (C₂-C₆)alkenyl, (C₃-C₈)cycloalkyl or (C₂-C₆)alkynyl radicals are optionally replaced independently of one another by F atoms;
R₁₂ and R₁₃ are defined independently of one another by H, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, (C₂-C₆)alkynyl, —C(O)—W—(C₁-C₆)alkyl, —C(O)—W—(C₂-C₆)alkenyl, —C(O)—W—(C₃-C₈)cycloalkyl, —C(O)—W—(C₁-C₄)alkyl-(C₃-C₈)cycloalkyl, —C(O)—W—(C₃-C₈)cycloalkyl-(C₁-C₄)alkyl, or —C(O)—W—(C₂-C₆)alkynyl;
W is a covalent bond, —O— or —NH—;
provided that
R₁, R₂ and R₃ are H,
A is C(O)OH,
n is 1,
L is a covalent bond, and
R₄ is a 4-(4′-chlorobiphenyl radical),
are not concurrently present;
R₁ and R₂ are O-methyl,
R₃ is H,
A is C(O)OH,
n is 1,
L is a covalent bond, and
R₄ is a 4-(4′-chlorobiphenyl radical),
are not concurrently present;
R₁ is OH or O-benzyl,
R₂ is H or O-methyl,
R₃ is H,
A is C(O)NHOH,
n is 1,
L is a covalent bond, and
R₄ is a phenyl radical which is unsubstituted or substituted by methyl, methoxy, NH₂, NO₂ or Cl,
are not concurrently present; and
R₁ and R₂ are H, OH or O(C₁-C₆)alkyl,
R₃ is H,
A is C(O)NHOH,
n is 1,
L is a covalent bond, and
R₄ is a phenyl radical which is unsubstituted or substituted by H, OH, O(C₁-C₆)alkyl or O(C₁-C₁₂)aryl, Br, C₁, NO₂, NH₂, (C₁-C₆)alkyl or (C₁-C₁₂)aryl,
are not concurrently present;
or a pharmacologically acceptable salt thereof.

2. The compound according claim 1, wherein
n is 1; and
R₄ is a pyridyl radical, which is optionally substituted by 1, 2 or 3 substitutents independently of one another selected from the group of F, Cl, Br, I, CN, OH, NO₂, (C₁-C₆)alkyl, (C₂-C₆)alkenyl, (C₂-C₆)alkynyl, (C₃-C₈)cycloalkyl, (C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₂-C₆)alkynyl, O(C₃-C₈)cycloalkyl, O(C₁-C₄)alkyl-(C₃-C₈)cycloalkyl, O(C₃-C₈)cycloalkyl-(C₁-C₄)alkyl, —O(C₁-C₄)alkyl-O—(C₁-C₆)alkyl or a radical NR₈R₉, and is substituted by a group T-Z;
or a pharmacologically acceptable salt thereof.

3. The compound according to claim 1, wherein
R₁, R₂ and R₃ are independently of one another H, F, Cl, Br, OH, NO₂, (C₁-C₆)alkyl, or —O(C₁-C₆)alkyl;
A is —C(O)NHOH;
L is a covalent bond or —(CH₂)_{q}—;
q is 1 or 2;
R₄ is phenyl or pyridyl, each of which is unsubstituted or substituted by 1, 2 or 3 radicals selected from the group of F, Cl, (C₁-C₆)alkyl, or —O(C₁-C₆)alkyl, and each of which is either substituted by NR₈R₉ or substituted by a group T-Z;
R₈ and R₉ are independently of one another H or (C₁-C₆)alkyl;
T is a covalent bond, —O—, —S—, —O—(C₁-C₄)alkyl- or —O—C(O)—;
Z is phenyl, (C₅-C₁₀)heteroaryl, (C₅-C₇)-heterocycloalkyl, or benzocyclo(C₅-C₇)alken-1-on-yl, each of which is unsubstituted or substituted by 1, 2 or 3 substitutents independently of one another selected from the group of F, Cl, Br, CN, OH, (C₁-C₆)alkyl, —SO₂(C₁-C₆)alkyl, —O(C₁-C₆)alkyl, —CH₂—C(O)—O(C₁-C₆)alkyl, —O—(C₁-C₄)alkyl-O—(C₁-C₆)alkyl, or NR₁₂R₁₃, wherein one or more H atoms in (C₁-C₆)alkyl or —O(C₁-C₆)alkyl may be replaced by F atoms;
R₁₂ and R₁₃ are independently of one another H, C(O)—W—(C₁-C₆)alkyl, or C(O)—W—(C₂-C₆)alkynyl;
W is a covalent bond or —O—; and
n is 1,
provided that
R₁ and R₂ are independently H, OH or O(C₁-C₆)alkyl,
R₃ is H,
A is C(O)NHOH,
n is 1,
L is a covalent bond, and
R₄ is a phenyl radical which is substituted by —O(C₁-C₁₂)aryl, —NH₂ or (C₁-C₁₂)aryl, and is optionally substituted by —O(C₁-C₆)alkyl,
(C₁-C₆)alkyl or Cl,
are not concurrently present;
or a pharmacologically acceptable salt thereof.

4. The compound according to claim 3, wherein
R₄ is a pyridyl radical, wherein the pyridyl radical is unsubstituted or substituted by 1, 2 or 3 radicals independently of one another selected from the group of F, Cl, (C₁-C₆)alkyl, or —O(C₁-C₆)alkyl, and wherein the pyridyl radical is either substituted by NR₈R₉ or substituted by a group T-Z; and
T is a covalent bond, —O—, —S—, —O—CH₂—, or —O—C(O)—;
or a pharmacologically acceptable salt thereof.

5. The compound according to claim 3, wherein
R₄ is phenyl or pyridyl, each of which is unsubstituted or substituted by 1, 2 or 3 radicals selected from the group of F, Cl, methyl or ethyl, or —O-methyl, and each of which is either substituted by —N(CH₃)₂ or substituted by a group T-Z;
T is a covalent bond, —O—, —S—, —O—CH₂—, or —O—C(O)—; and
Z is phenyl, pyridyl, pyrazolyl, indolyl, morpholinyl, or indan-1-on-yl, each of which is unsubstituted or substituted by 1, 2 or 3 substitutents independently of one another selected from the group of F, Cl, Br, CN, OH, —CF₃, —SO₂CH₃, —OMe, —OEt, —O(CH₂)₃CH₃, —OCF₃, —OCH₂CF₃, —CH₂—C(O)—OMe, —CH₂—C(O)—OEt, —O(CH₂)₂OCH₃, —NHC(O)O(i-Pr), —NHC(O)OCH₂C≡CCH₃ or —NHC(O)CH₃;
or a pharmacologically acceptable salt thereof.

6. The compound according to claim 5, wherein
R4 is pyridyl, wherein the pyridyl is unsubstituted or substituted by 1, 2 or 3 radicals selected from the group of F, Cl, methyl or ethyl, or —O-methyl, and wherein the pyridyl is either substituted by —N(CH₃)₂ or substituted by a group T-Z;
or a pharmacologically acceptable salt thereof.

7. The compound according to claim 1, wherein
R₁, R₂ and R₃ are independently of one another H, F, NO₂, (C₁-C₆)alkyl, or —O(C₁-C₆)alkyl;
A is C(O)NHOH,
L is a covalent bond or —(CH₂)_{q}—;
q is 1 or 2;
R₄ is phenyl or pyridyl, each of which is substituted by a radical T-Z;
T is a covalent bond or —O—;
Z is phenyl or pyridyl, each of which is unsubstituted or substituted by 1, 2 or 3 substitutents selected independently of one another from the group of F, Cl or Br, —O(C₁-C₆)alkyl, or NR₁₂R₁₃, wherein one or more H atoms in —O(C₁-C₆)alkyl may be replaced by F atoms;
R₁₂ and R₁₃ are independently of one another H, or C(O)—O—(C₁-C₆)alkyl; and
n is 1;
or a pharmacologically acceptable salt thereof.

8. The compound according to claim 7, wherein:
R₁, R₂ and R₃ are independently of one another H, F, NO₂, methyl, ethyl, or —O-methyl; and
Z is phenyl or pyridyl, each of which is unsubstituted or substituted by 1, 2 or 3 substitutents selected independently of one another from the group of F, Cl or Br, —O-methyl, O-ethyl, —OCF₃, —OCH₂CF₃ or NR₁₂R₁₃;
or a pharmacologically acceptable salt thereof.

9. The compound according to claim 7, where
R₄ is pyridyl substituted by a radical T-Z;
or a pharmacologically acceptable salt thereof.

10. The compound according to claim 8, wherein
R₄ is pyridyl substituted by a radical T-Z;
or a pharmacologically acceptable salt thereof.

11. The compound according to claim 1, having formula (II) 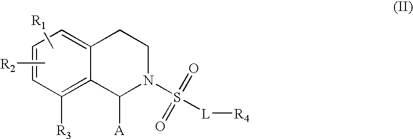
R₃ is F, Cl, Br, I, NO₂, CN, OH, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, —O(C₁-C₆)alkyl, O(C₂-C₆)alkenyl, —O(C₃-C₈)cycloalkyl, —O(C₁-C₄)alkyl-(C₃-C₈)cycloalkyl, —O(C₃-C₈)cycloalkyl-(C₁-C₄)alkyl, —OC(O)—(C₁-C₆)alkyl, —OC(O)—(C₂-C₆)alkenyl, —OC(O)—(C₃-C₈)cycloalkyl, —OC(O)—(C₁-C₄)alkyl-(C₃-C₈)cycloalkyl, —OC(O)—(C₃-C₈)cycloalkyl-(C₁-C₄)alkyl, —C(O)O—(C₁-C₆)alkyl, —C(O)O—(C₂-C₆)alkenyl, —C(O)O—(C₃-C₉)cycloalkyl, —C(O)O—(C₁-C₄)alkyl-(C₃-C₈)cycloalkyl, —C(O)O—(C₃-C₈)cycloalkyl-(C₁-C₄)alkyl, —C(O)NR₆R₇, —NR₆R₇ or —NR₆C(O)R₇;
or a pharmacologically acceptable salt thereof.

12. The compound according to claim 11, wherein
R₄ is pyridyl, wherein the pyridyl is optionally substituted by 1, 2 or 3 substitutents independently of one another selected from the group of F, Cl, Br, I, CN, OH, NO₂, (C₁-C₆)alkyl, (C₂-C₆)alkenyl, (C₂-C₆)alkynyl, (C₃-C₈)cycloalkyl, (C₁-C₄)alkyl-(C₃-C₈)cycloalkyl, —(C₃-C₈)cycloalkyl-(C₁-C₄)alkyl, —O(C₁-C₆)alkyl, O(C₂-C₆)alkenyl, —O(C₂-C₆)alkynyl, —O(C₃-C₈)cycloalkyl, —O(C₁-C₄)alkyl-(C₃-C₈)cycloalkyl, —O(C₃-C₈)cycloalkyl-(C₁-C₄)alkyl, —O(C₁-C₄)—O—(C₁-C₆) or a radical NR₈R₉, and wherein the pyridyl is optionally substituted by a group T-Z;
or a pharmacologically acceptable salt thereof.

13. The compound according to claim 11, wherein
R₁ and R₂ are independently of one another H, F, Cl, Br, NO₂, CN, OH, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, —O(C₁-C₆)alkyl, —O(C₂-C₆)alkenyl, —O(C₃-C₈)cycloalkyl, —O—(C₁-C₄)alkyl-(C₃-C₈)cycloalkyl or —O—(C₃-C₈)cycloalkyl-(C₁-C₄)alkyl;
R₃ is F, Cl, Br, NO₂, CN, OH, (C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, O(C₁-C₆)alkyl, O(C₂-C₆)alkenyl, O(C₃-C₈)cycloalkyl, —O—(C₁-C₄)alkyl-(C₃-C₈)cycloalkyl or —O—(C₃-C₈)cycloalkyl-(C₁-C₄)alkyl;
R₅ is OH, NH₂ or NHOH,
L is a covalent bond or —(CH₂)_{q}—;
q is 1 or 2;
R₄ is phenyl or pyridyl, each of which is optionally substituted by 1, 2 or 3 substitutents independently of one another selected from the group of F, Cl, Br, CN, OH, NO₂, (C₁-C₆)alkyl, —O—(C₁-C₆)alkyl, (C₂-C₆)alkenyl, (C₃-C₈)cycloalkyl, —(C₁-C₄)alkyl-(C₃-C₈)cycloalkyl, —(C₃-C₈)cycloalkyl-(C₁-C₄)alkyl, —O(C₂-C₆)alkenyl, —O(C₃-C₈)cycloalkyl, —O—(C₁-C₄)alkyl-(C₃-C₈)cycloalkyl, —O—(C₃-C₈)cycloalkyl-(C₁-C₄)alkyl, (C₂-C₆)alkynyl, —O(C₂-C₆)alkynyl, or NR₈R₉, and each of which is substituted by a group T-Z;
R₈ and R₉ are independently of one another H or (C₁-C₆)alkyl;
T is a covalent bond or —O—;
Z is phenyl, (C₅-C₁₀)heteroaryl, C₅-C₇-heterocycloalkyl, or benzocyclo(C₅-C₇)alken-1-on-yl, each of which is unsubstituted or substituted by 1, 2 or 3 substitutents independently of one another selected from the group of F, Cl, Br, CN, OH, (C₁-C₆)alkyl, —SO₂(C₁-C₆)alkyl, —O(C₁-C₆)alkyl, —CH₂—C(O)—O(C₁-C₆)alkyl, —O—(C₁-C₄)alkyl-O—(C₁-C₆)alkyl, or NR₁₂R₁₃, wherein one or more H atoms in (C₁-C₆)alkyl or —O(C₁-C₆)alkyl may be replaced by F atoms;
R₁₂ and R₁₃ are independently of one another H, C(O)—W—(C₁-C₆)alkyl, or C(O)—W—(C₂-C₆)alkynyl; and
W is a covalent bond or —O—;
or a pharmacologically acceptable salt thereof.

14. The compound according to claim 13, wherein:
Z is phenyl, pyridyl, pyrazolyl, indolyl, morpholinyl, or indan-1-on-yl, each of which is unsubstituted or substituted by 1, 2 or 3 substitutents independently of one another selected from the group of F, Cl, Br, CN, OH, —CF₃, —SO₂CH₃, —OMe, —OEt, —O(CH₂)₃CH₃, —OCF₃ or —OCH₂CF₃, —CH₂—C(O)—OMe, —CH₂—C(O)—OEt, —O(CH₂)₂OCH₃, —NHC(O)O(i-Pr), —NHC(O)OCH₂C≡CCH₃ or —NHC(O)CH₃;
or a pharmacologically acceptable salt thereof.

15. The compound according to claim 11, wherein
R₁ and R₂ are independently of one another H or (C₁-C₆)alkyl;
R₃ is F, Cl, Br, (C₁-C₆)alkyl or O(C₁-C₆)alkyl;
A is C(O)NHOH;
L is a covalent bond or —(CH₂)_{q}—;
q is 1 or 2,
R₄ is phenyl or pyridyl, each of which is optionally substituted by 1, 2 or 3 radicals independently of one another selected from the group of F, Cl, NO₂, (C₁-C₆)alkyl, —O(C₁-C₆)alkyl, and each of which is further substituted by a group T-Z;
T is a covalent bond, —O—, —S—, —O—(C₁-C₄)alkyl-, or —O—C(O)—; and
Z is phenyl or pyridyl, each of which is unsubstituted or substituted by 1, 2 or 3 substitutents independently of one another selected from the group of F, Cl, or —O(C₁-C₆)alkyl, where one or more H atoms in —O(C₁-C₆)alkyl may be replaced by F atoms;
or a pharmacologically acceptable salt thereof.

16. The compound according to claim 15, wherein:
R₄ is phenyl or pyridyl, each of which is optionally substituted by 1, 2 or 3 radicals independently of one another selected from the group of F, Cl, NO₂, methyl, ethyl, or —O-methyl, and each of which is further substituted by a group T-Z;
T is a covalent bond, —O—, —S—, —O—CH₂— or —O—C(O)—; and
Z is phenyl or pyridyl, each of which is unsubstituted or substituted by 1, 2 or 3 substitutents independently of one another selected from the group of F, Cl, —O-methyl, —O-ethyl or —OCF₃;
or a pharmacologically acceptable salt thereof.

17. The compound according to claim 11, wherein
R₁ and R₂ are independently of one another H, methyl or ethyl;
R₃ is F or —O-methyl;
A is C(O)NHOH;
R₄ is phenyl or pyridyl, each of which is substituted by a radical T-Z;
T is a covalent bond or —O—;
Z is phenyl or pyridyl, where each of which is unsubstituted or substituted by 1, 2 or 3 substitutents selected independently of one another from the group of F, Cl, Br, or —O(C₁-C₆)alkyl, where one or more H atoms in (C₁-C₆)alkyl are optionally replaced independently of one another by F atoms; and
L is a covalent bond;
or a pharmacologically acceptable salt thereof.

18. The compound according to claim 17, wherein:
Z is phenyl or pyridyl, where each of which is unsubstituted or substituted by 1, 2 or 3 substitutents selected independently of one another from the group of F, Cl, —O-methyl, —O-ethyl, or —OCF₃;
or a pharmacologically acceptable salt thereof.

19. The compound according to claim 1, which is
isopropyl[4′-(1-hydroxycarbamoyl-3,4-dihydro-1H-isoquinoline-2-sulfonyl)-biphenyl-4-yl]carbamate;
but-2-ynyl[4′-(1-hydroxycarbamoyl-3,4-dihydro-1H-isoquinoline-2-sulfonyl)-biphenyl-4-yl]carbamate;
2-(6-phenoxypyridine-3-sulfonyl)-1,2,3,4-tetrahydroisoquinoline-1-hydroxycarboxamide;
2-(6-morpholin-4-ylpyridine-3-sulfonyl)-1,2,3,4-tetrahydroisoquinoline-1-hydroxycarboxamide;
2-(2-biphenyl-4-ylethanesulfonyl)-1,2,3,4-tetrahydroisoquinoline-1-hydroxycarboxamide;
2-(4-pyrazol-1-ylbenzenesulfonyl)-1,2,3,4-tetrahydroisoquinoline-1-hydroxycarboxamide;
but-2-ynyl(R)-[4′-(1-hydroxycarbamoyl-3,4-dihydro-1H-isoquinoline-2-sulfonyl)-biphenyl-4-yl]carbamate;
but-2-ynyl(S)-[4′-(1-hydroxycarbamoyl-3,4-dihydro-1H-isoquinoline-2-sulfonyl)-biphenyl-4-yl]carbamate;
(R)-2-(4-pyrazol-1-ylbenzenesulfonyl)-1,2,3,4-tetrahydroisoquinoline-1-hydroxycarboxamide;
(S)-2-(4-pyrazol-1-ylbenzenesulfonyl)-1,2,3,4-tetrahydroisoquinoline-1-hydroxycarboxamide;
(R)-2-(6-phenoxypyridine-3-sulfonyl)-1,2,3,4-tetrahydroisoquinoline-1-hydroxycarboxamide;
(S)-2-(6-phenoxypyridine-3-sulfonyl)-1,2,3,4-tetrahydroisoquinoline-1-hydroxycarboxamide;
but-2-ynyl {4-[2-(1-hydroxycarbamoyl-3,4-dihydro-1H-isoquinoline-2-sulfonyl)-ethyl]phenyl}carbamate;
(R)-2-(2-biphenyl-4-ylethanesulfonyl)-1,2,3,4-tetrahydroisoquinoline-1-hydroxycarboxamide;
(S)-2-(2-biphenyl-4-ylethanesulfonyl)-1,2,3,4-tetrahydroisoquinoline-1-hydroxycarboxamide;
but-2-ynyl(R)-{4-[2-(1-hydroxycarbamoyl-3,4-dihydro-1H-isoquinoline-2-sulfonyl)ethyl]phenyl}carbamate;
but-2-ynyl(S)-{4-[2-(1-hydroxycarbamoyl-3,4-dihydro-1H-isoquinoline-2-sulfonyl)ethyl]phenyl}carbamate;
(R)-2-(6-morpholin-4-ylpyridine-3-sulfonyl)-1,2,3,4-tetrahydroisoquinoline-1-hydroxycarboxamide;
(S)-2-(6-morpholin-4-ylpyridin-3-sulfonyl)-1,2,3,4-tetrahydroisoquinoline-1-hydroxycarboxamide;
2-{2-[4-(4-fluorophenoxy)phenyl]ethanesulfonyl}-1,2,3,4-tetrahydroisoquinoline-1-hydroxycarboxamide;
(R)-2-[4-(4-trifluoromethylphenoxy)benzenesulfonyl]-1,2,3,4-tetrahydro-isoquinoline-1-hydroxycarboxamide;
(R)-2-[4-(4-methoxyphenoxy)benzenesulfonyl]-1,2,3,4-tetrahydroisoquinoline-1-hydroxycarboxamide;
2-[2-(4′-chlorobiphenyl-4-yl)ethanesulfonyl]-1,2,3,4-tetrahydroisoquinoline-1-hydroxycarboxamide;
(R)-2-[4-(4-chlorophenoxy)benzenesulfonyl]-1,2,3,4-tetrahydroisoquinoline-1-hydroxycarboxamide;
(R)-2-[4-(pyridin-2-yloxy)benzenesulfonyl]-1,2,3,4-tetrahydroisoquinoline-1-hydroxycarboxamide;
2-[4-(pyridin-4-yloxy)benzenesulfonyl]-1,2,3,4-tetrahydroisoquinoline-1-hydroxycarboxamide;
2-[6-(4-chlorophenoxy)pyridine-3-sulfonyl]-1,2,3,4-tetrahydroisoquinoline-1-hydroxycarboxamide;
2-[2-(4′-chlorobiphenyl-4-yl)ethanesulfonyl]-6-nitro-1,2,3,4-tetrahydro-isoquinoline-1-hydroxycarboxamide;
(R)-2-[2-(4′-chlorobiphenyl-4-yl)ethanesulfonyl]-1,2,3,4-tetrahydroisoquinoline-1-hydroxycarboxamide;
(S)-2-[2-(4′-chlorobiphenyl-4-yl)ethanesulfonyl]-1,2,3,4-tetrahydroisoquinoline-1-hydroxycarboxamide;
(R)-2-[4-(4-methanesulfonylphenoxy)benzenesulfonyl]-1,2,3,4-tetrahydro-isoquinoline-1-hydroxycarboxamide;
(R)-2-[4-(4-methanesulfonylphenoxy)-2-methylbenzenesulfonyl]-1,2,3,4-tetrahydroisoquinoline-1-hydroxycarboxamide;
2-(4′-chlorobiphenyl-4-sulfonyl)-6,7-dimethoxy-1,2,3,4-tetrahydroisoquinoline-1-hydroxycarboxamide;
(R)-2-(4′-chlorobiphenyl-4-sulfonyl)-1,2,3,4-tetrahydroisoquinoline-1-hydroxycarboxamide;
2-[6-(4-cyanophenoxy)pyridine-3-sulfonyl]-1,2,3,4-tetrahydroisoquinoline-1-hydroxycarboxamide;
methyl {4-[5-(1-hydroxycarbamoyl-3,4-dihydro-1H-isoquinoline-2-sulfonyl)pyridine-2-yloxy]phenyl}acetate;
2-[6-(3-acetylaminophenoxy)pyridine-3-sulfonyl]-1,2,3,4-tetrahydroisoquinoline-1-hydroxycarboxamide;
ethyl {4-[5-(1-hydroxycarbamoyl-3,4-dihydro-1H-isoquinoline-2-sulfonyl)pyridine-2-yloxy]phenyl}acetate;
2-[6-(4-cyano-3-fluorophenoxy)pyridine-3-sulfonyl]-1,2,3,4-tetrahydro-isoquinoline-1-hydroxycarboxamide;
2-[6-(1-oxoindan-5-yloxy)pyridine-3-sulfonyl]-1,2,3,4-tetrahydroisoquinoline-1-hydroxycarboxamide;
2-[6-(2-methyl-1H-indol-5-yloxy)pyridine-3-sulfonyl]-1,2,3,4-tetrahydro-isoquinoline-1-hydroxycarboxamide;
(R)-2-[6-(4-methoxyphenoxy)pyridine-3-sulfonyl]-1,2,3,4-tetrahydroisoquinoline-1-hydroxycarboxamide;
(R)-2-[6-(4-trifluoromethoxyphenoxy)pyridine-3-sulfonyl]-1,2,3,4-tetrahydro-isoquinoline-1-hydroxycarboxamide;
(R)-2-{4-[4-(2,2,2-trifluoroethoxy)phenoxy]benzenesulfonyl}-1,2,3,4-tetrahydro-isoquinoline-1-hydroxycarboxamide;
(R)-2-[6-(4-chlorophenoxy)pyridine-3-sulfonyl]-1,2,3,4-tetrahydroisoquinoline-1-hydroxycarboxamide;
(R)-2-[6-(4-ethoxyphenoxy)pyridine-3-sulfonyl]-1,2,3,4-tetrahydroisoquinoline-1-hydroxycarboxamide;
(R)-2-(4-phenylsulfanylbenzenesulfonyl)-1,2,3,4-tetrahydroisoquinoline-1-hydroxycarboxamide;
(R)-4-(1-hydroxycarbamoyl-3,4-dihydro-1H-isoquinoline-2-sulfonyl)phenyl benzoate;
(R)-2-[4-(4-fluorobenzyloxy)benzenesulfonyl]-1,2,3,4-tetrahydroisoquinoline-1-hydroxycarboxamide;
(R)-2-[4′-(2,2,2-trifluoroethoxy)biphenyl-4-sulfonyl]-1,2,3,4-tetrahydro-isoquinoline-1-hydroxycarboxamide;
(R)-2-{6-[4-(2-methoxyethoxy)phenoxy]pyridine-3-sulfonyl}-1,2,3,4-tetrahydro-isoquinoline-1-hydroxycarboxamide;
(R)-2-[4-(4-hydroxyphenoxy)benzenesulfonyl]-1,2,3,4-tetrahydroisoquinoline-1-hydroxycarboxamide;
(S)-2-[6-(4-ethoxyphenoxy)pyridine-3-sulfonyl]-1,2,3,4-tetrahydroisoquinoline-1-hydroxycarboxamide;
2-[4-(4-ethoxyphenoxy)-3-fluorobenzenesulfonyl]-1,2,3,4-tetrahydroisoquinoline-1-hydroxycarboxamide;
(R)-2-[4-(4-ethoxyphenoxy)-3-fluorobenzenesulfonyl]-1,2,3,4-tetrahydro-isoquinoline-1-hydroxycarboxamide;
2-(6-butoxypyridine-3-sulfonyl)-1,2,3,4-tetrahydroisoquinoline-1-hydroxycarboxamide;
(R)-2-[4-(4-fluorophenoxy)benzenesulfonyl]-1,2,3,4-tetrahydroisoquinoline-1-hydroxycarboxamide;
(S)-2-[4-(4-fluorophenoxy)benzenesulfonyl]-1,2,3,4-tetrahydroisoquinoline-1-hydroxycarboxamide;
2-[6-(4-trifluoromethoxyphenoxy)pyridine-3-sulfonyl]-1,2,3,4-tetrahydro-isoquinoline-1-hydroxycarboxamide;
2-(6-dimethylaminopyridine-3-sulfonyl)-1,2,3,4-tetrahydroisoquinoline-1-hydroxycarboxamide;
2-{3-fluoro-4-[4-(2-methoxyethoxy)phenoxy]benzenesulfonyl}-1,2,3,4-tetrahydroisoquinoline-1-hydroxycarboxamide;
2-[3-chloro-4-(4-ethoxyphenoxy)benzenesulfonyl]-1,2,3,4-tetrahydroisoquinoline-1-hydroxycarboxamide;
2-[4-(4-ethoxyphenoxy)benzenesulfonyl]-1,2,3,4-tetrahydroisoquinoline-1-hydroxycarboxamide;
(R)-2-[4-(4-trifluoromethoxyphenoxy)benzenesulfonyl]-1,2,3,4-tetrahydro-isoquinoline-1-hydroxycarboxamide;
(S)-2-[4-(4-trifluoromethoxyphenoxy)benzenesulfonyl]-1,2,3,4-tetrahydro-isoquinoline-1-hydroxycarboxamide;
(R)-2-[4-(4-ethoxyphenoxy)benzenesulfonyl]-1,2,3,4-tetrahydroisoquinoline-1-hydroxycarboxamide;
(S)-2-[4-(4-ethoxyphenoxy)benzenesulfonyl]-1,2,3,4-tetrahydroisoquinoline-1-hydroxycarboxamide;
6-hydroxy-2-[4-(4-methoxyphenoxy)benzenesulfonyl]-1,2,3,4-tetrahydro-isoquinoline-1-hydroxycarboxamide;
(R)-2-{3-fluoro-4-[4-(2-methoxyethoxy)phenoxy]benzenesulfonyl}-1,2,3,4-tetrahydroisoquinoline-1-hydroxycarboxamide; or
(S)-2-{3-fluoro-4-[4-(2-methoxyethoxy)phenoxy]benzenesulfonyl}-1,2,3,4-tetrahydroisoquinoline-1-hydroxycarboxamide;
a pharmacologically acceptable salt thereof.

20. The compound according to claim 11, which is
8-fluoro-5-methyl-2-(6-phenoxypyridine-3-sulfonyl)-1,2,3,4-tetrahydro-isoquinoline-1-hydroxycarboxamide;
2-(4′-chlorobiphenyl-4-sulfonyl)-5-fluoro-8-methoxy-1,2,3,4-tetrahydro-isoquinoline-1-hydroxycarboxamide;
2-(4′-chlorobiphenyl-4-sulfonyl)-8-fluoro-5-methyl-1,2,3,4-tetrahydro-isoquinoline-1-hydroxycarboxamide;
5-ethyl-8-fluoro-2-(6-phenoxypyridine-3-sulfonyl)-1,2,3,4-tetrahydro-isoquinoline-1-hydroxycarboxamide;
2-(4′-chlorobiphenyl-4-sulfonyl)-5-ethyl-8-fluoro-1,2,3,4-tetrahydroisoquinoline-1-hydroxycarboxamide;
8-fluoro-2-[6-(4-methoxyphenoxy)pyridine-3-sulfonyl]-5-methyl-1,2,3,4-tetrahydroisoquinoline-1-hydroxycarboxamide;
8-fluoro-5-methyl-2-[6-(4-trifluoromethoxyphenoxy)pyridine-3-sulfonyl]-1,2,3,4-tetrahydroisoquinoline-1-hydroxycarboxamide;
2-[4-(4-chlorophenoxy)benzenesulfonyl]-8-fluoro-5-methyl-1,2,3,4-tetrahydro-isoquinoline-1-hydroxycarboxamide;
8-fluoro-5-methyl-2-[4-(pyridin-4-yloxy)benzenesulfonyl]-1,2,3,4-tetrahydro-isoquinoline-1-hydroxycarboxamide;
8-fluoro-5-ethyl-2-[4-(pyridin-4-yloxy)benzenesulfonyl]-1,2,3,4-tetrahydro-isoquinoline-1-hydroxycarboxamide;
2-[6-(4-ethoxyphenoxy)pyridine-3-sulfonyl]-8-fluoro-5-methyl-1,2,3,4-tetrahydroisoquinoline-1-hydroxycarboxamide;
2-[4-(4-chlorophenoxy)benzenesulfonyl]-5-ethyl-8-fluoro-1,2,3,4-tetrahydro-isoquinoline-1-hydroxycarboxamide;
(R)-2-[4-(4-chlorophenoxy)benzenesulfonyl]-5-methyl-8-fluoro-1,2,3,4-tetrahydroisoquinoline-1-hydroxycarboxamide;
(S)-2-[4-(4-chlorophenoxy)benzenesulfonyl]-5-methyl-8-fluoro-1,2,3,4-tetrahydroisoquinoline-1-hydroxycarboxamide;
(R)-2-[6-(4-ethoxyphenoxy)pyridine-3-sulfonyl]-5-methyl-8-fluoro-1,2,3,4-tetrahydroisoquinoline-1-hydroxycarboxamide;
(S)-2-[6-(4-ethoxyphenoxy)pyridine-3-sulfonyl]-5-methyl-8-fluoro-1,2,3,4-tetrahydroisoquinoline-1-hydroxycarboxamide;
5-methyl-2-[4-(4-fluorophenoxy)benzenesulfonyl]-8-fluoro-1,2,3,4-tetrahydro-isoquinoline-1-hydroxycarboxamide;
(R)-5-methyl-2-[4-(4-fluorophenoxy)benzenesulfonyl]-8-fluoro-1,2,3,4-tetrahydroisoquinoline-1-hydroxycarboxamide; or
(S)-5-methyl-2-[4-(4-fluorophenoxy)benzenesulfonyl]-8-fluoro-1,2,3,4-tetrahydroisoquinoline-1-hydroxycarboxamide;
or a pharmacologically acceptable salt thereof.

21. A pharmaceutical composition comprising a pharmaceutically effective amount of the compound according to claim 1 or a pharmacologically acceptable salt thereof, and a physiologically tolerated excipient or carrier.

22. A method for treating a cardiovascular disorder, inflammation, cancer, tumor metastasis, cachexia, anorexia, ulceration, a degenerative joint disorder, a connective tissue disorder, a periodontal disorder, a wound-healing disturbance, or a chronic disorder of locomotor system, or for the prophylaxis of myocardial or cerebral infarction, in a patient in need thereof, comprising administering to the patient a pharmaceutically effective amount of the compound according to claim 1 or pharmacologically acceptable salt thereof.

23. The method according to claim 22, wherein the cardiovascular disorder is remodeling of the heart after a myocardial infarction or atherosclerosis, unstable angina pectoris, heart failure, stenosis or septic shock.

24. The method according to claim 22, wherein the degenerative joint disorder is osteoarthrosis, spondylosis, or chondrolysis after joint trauma, prolonged joint immobilization after meniscus, r patella injuries or ligament tear.

25. The method according to claim 22, wherein the connective tissue disorder is collagenosis.

26. The method according to claim 22, wherein the chronic disorder of locomotor system is inflammatory, immunologically or metabolism-related acute or chronic arthritis, arthropathy, myalgias or disturbance of bone metabolism.
